SEQUENCE LISTING (1 sequence)

SEQ ID NO: 1 - protein, 413 aa, Homo sapiens [sequence omitted]

(19) United States
(12) Patent Application Publication
(10) Pub. No.: US 2024/0166626 A1
(43) Pub. Date: May 23, 2024

(54) Compounds and Methods for Preventing, Treating, or Ameliorating Airway Disease

(71) Applicants: THOMAS JEFFERSON UNIVERSITY, Philadelphia, PA (US); THE WISTAR INSTITUTE, Philadelphia, PA (US)

(72) Inventors: Michael Joseph IPPOLITO, Philadelphia, PA (US); Nathan Ross HOPFINGER, Ardmore, PA (US); Charles P. SCOTT, Wynnewood, PA (US); Roger S. ARMEN, Philadelphia, PA (US); Jeffrey Lee BENOVIC, Philadelphia, PA (US); Joseph M. Salvino, Chester Springs, PA (US)

(21) Appl. No.: 18/274,514

(22) PCT Filed: Feb. 8, 2022
(86) PCT No.: PCT/US22/15650

§ 371 Date: Jul. 27, 2023

Related U.S. Application Data
(60) Provisional application No. 63/147,380, filed on Feb. 9, 2021.

(51) Int. Cl. C07D 401/12; C07D 239/95

(52) U.S. Cl. CPC C07D 401/12; C07D 239/95

(57) ABSTRACT

The β2-adrenergic receptor (β2AR) is a primary target in the treatment of airway diseases such as asthma and chronic obstructive pulmonary disease (COPD). Since β-agonist treatment of airway disease can have severe side effects, compounds that attenuate the side effects of β-agonists have been identified via their ability to inhibit β2AR interaction with β-arrestins. These compounds are specific for the β2AR and effectively protect against the desensitization observed with β-agonist treatment in model cells and airway tissue. The present disclosure provides compounds and methods for treating airway disease, such as but not limited to COPD and/or asthma.

### CROSS REFERENCE TO RELATED APPLICATIONS

The present application claims priority under 35 U.S.C. § 119(e) to U.S. Provisional Patent Application No. 63/147,380, filed Feb. 9, 2021, which application is incorporated herein by reference in its entirety.

### STATEMENT REGARDING FEDERALLY SPONSORED RESEARCH OR DEVELOPMENT

This invention was made with government support under P01 HL114471 and F31 HL139104-01A1 awarded by National Institutes of Health. The government has certain rights in the invention.

### SEQUENCE LISTING

The ASCII text file named “205961-7027WO1-Sequence Listing” created on Jan. 25, 2022, comprising 4.0 Kbytes, is hereby incorporated by reference in its entirety.

### BACKGROUND

The β2-adrenergic receptor (β₂AR) is an extensively investigated G protein-coupled receptor (GPCR) that mediates cyclic adenosine monophosphate (cAMP) production in response to catecholamines such as epinephrine and norepinephrine. β₂AR signaling coordinates important physiological functions including airway and uterine smooth muscle relaxation. The role of β₂AR signaling in airway relaxation has made the receptor a clinically important therapeutic target for airway diseases such as asthma and chronic obstructive pulmonary disease (COPD). Currently, β₂AR agonists are among the most commonly prescribed drugs, and remain the most potent promoters of airway relaxation approved for clinical use.

Asthma is a common chronic disorder characterized by airway inflammation and airway smooth muscle (ASM) contraction leading to pathologic airway constriction. Asthma-related inflammation damages airway epithelial cells and sensitizes ASM to constriction triggers. β-agonists are cornerstone therapies for asthma, but long-term use of these drugs can lead to a paradoxical loss of therapeutic response, which can indeed promote severe adverse effects and an increased risk of sudden death during an asthma attack. Despite continued drug development, harmful side effects persist in new generations of β-agonists. This is due in part to a failure of discovery efforts to integrate new information about the dynamic nature of β₂AR signaling modalities into screening efforts, as well as the reliance on conventional GPCR drug discovery strategies that often investigate only the endogenous ligand binding or orthosteric site. While many novel small molecule β₂AR ligands have been discovered, screening efforts have not sufficiently attended to the underlying signaling properties that lead to unwanted side effects.

There is thus a need in the art for novel compounds and compositions that can be used to treat and/or prevent and/or ameliorate asthma. The present disclosure addresses this need.

### BRIEF SUMMARY

In one aspect, the disclosure provides compounds that act as β2-adrenergic receptor-specific allosteric modulators. In some embodiments, the β₂AR allosteric modulator is a compound of formula (1), formula (2), and/or formula (3), or a pharmaceutically acceptable salt, solvate, isotopologue, stereoisomer, tautomer, and/or any mixture thereof.

In another aspect, the disclosure provides a method of preventing, treating, and/or ameliorating airway disease in a subject. In certain embodiments, the method comprises administering to the mammal a therapeutically effective amount of a compound contemplated within the scope of the present disclosure. In certain embodiments, the airway disease is asthma or chronic obstructive pulmonary disease (COPD).

### BRIEF DESCRIPTION OF THE DRAWINGS

The following detailed description of illustrative embodiments of the disclosure will be better understood when read in conjunction with the appended drawings. For the purpose of illustrating the disclosure, certain illustrative embodiments are shown in the drawings. It should be understood, however, that the disclosure is not limited to the precise arrangements and instrumentalities of the embodiments shown in the drawings.

FIGS. 1A-1E: Identifying small-molecule allosteric modulators of β-arrestin recruitment to the β2AR. FIG. 1A: Schematic of screening methodologies for cAMP production and β-arrestin binding. Cells were treated with the unbiased beta-agonist isoproterenol (ISO)±test compounds. The primary high throughput screen utilized a luciferase-based cAMP biosensor, GLOSENSOR™ (Promega), and an enzyme complementation-based assay, PATHHUNTER® (DiscoveRx). Independent secondary screening utilized a cAMP ELISA and BRET for β-arrestin recruitment. FIG. 1B: Secondary screen for cAMP production by ELISA. HEK 293 cells stably expressing β2AR were pre-incubated with 0.1% dimethyl sulfoxide (DMSO) (negative control) or 10 μM N⁴-cyclohexyl-N²-(3,4-difluorophenyl)quinazoline-2,4-diamine (DFPQ) for 30 min and then stimulated with 1 μM isoprenaline (ISO) for 10 min. Cells were lysed and cAMP production was measured. FIG. 1C: Dose response curve for DFPQ as measured by BRET. HEK 293 cells co-transfected with β-arrestin2-GFP10 and β₂AR-RlucII were pre-incubated with 0.1% DMSO (negative control) or the indicated concentrations of DFPQ for 30 min. Cells were incubated with Coelenterazine 400a for 2 min and then stimulated with 1 μM ISO. Data for dose response curve was taken 12 min post ISO addition. n=3. FIG. 1D: HEK 293 cells co-transfected with β-arrestin2-GFP10 and β₂AR-RLucII were pre-incubated with the indicated concentrations of DFPQ for 30 min. Cells were then incubated with Coelenterazine 400a for 2 min and then stimulated with the indicated concentrations of ISO. Data for dose response curves were taken 12 min post ISO addition. FIG. 1E: HEK 293 cells stably expressing β₂AR were pre-incubated with 0.1% DMSO (negative control), 1 μM DFPQ, or 10 μM DFPQ and stimulated with the indicated concentrations of ISO for 10 min. Cells were lysed and cAMP production was measured by ELISA. Data are normalized to 1 μM ISO.

FIGS. 2A-2C: GPCR specificity of DFPQ. HEK 293 cells were co-transfected with (3-arrestin2-GFP10 and either β₂AR-RlucII (FIG. 2A), (31AR-RlucII (FIG. 2B) or CXCR4-RlucII (FIG. 2C) for 48 h and cells were pre-incubated with 0.1% DMSO (negative control) or 10 μM DFPQ for 30 min. Cells were then incubated with Coelenterazine 400a for 2 min and stimulated with 1 μM of the indicated agonist. Cells were read every 2 min post agonist addition. Top panels show a time course for the BRET assay and bottom panels show the BRET signal at the 22 min time point. ****, p<0.001; ns, not significant.

FIGS. 3A-3D: DFPQ inhibits GRK-mediated phosphorylation of the β₂AR. FIG. 3A: Representative time course of agonist promoted phosphorylation of the β₂AR. HEK 293 cells stably expressing FLAG-β₂AR were pre-incubated with 0.1% DMSO or indicated concentrations of DFPQ for 30 min and then stimulated with 1 μM ISO for 10 min. Cells were lysed and the FLAG-β₂AR was immunoprecipitated. Phosphorylation at serine 355 and 356 was analyzed by western blot using a pSer^{355/356} antibody while total β₂AR was measured using a β₂AR C-terminal antibody. The western blot is representative of at least 3 experiments and is quantified in FIG. 3B. FIG. 3C: Purified GRK2 or GRK5 were incubated with tubulin, 10 μM DFPQ, and [γ³²P]-ATP for the indicated times. Reactions were stopped with SDS sample buffer and the samples were electrophoresed on 10% SDS-polyacrylamide gels and visualized by autoradiography. FIG. 3D: Bar graphs represent pixel densitometry of radiolabeled tubulin at the 60 min timepoint from 4 experiments.

FIGS. 4A-4F: DFPQ inhibits internalization of the β₂AR and protects from agonist induced desensitization in cell and tissue models. FIG. 4A: Effect of DFPQ on agonist-promoted internalization of the β₂AR. HEK 293 cells stably expressing FLAG-β₂AR were pre-incubated with 0.1% DMSO or 10 μM DFPQ for 30 min and then stimulated with the indicated concentrations of ISO for up to 60 min. Cells were fixed and receptor surface expression was measured by ELISA. These data represent the mean±SD from 3 independent experiments. ***, p<0.001; **, p<0.01; *, p<0.05 FIG. 4B: HEK 293 cells were desensitized by incubating with 1 μM ISO±10 μM DFPQ, washed with PBS, then stimulated with ISO at the indicated concentrations for 10 min. Cells were lysed and cAMP production was measured by ELISA. Cells incubated with DFPQ during desensitization were significantly protected from reduced response to agonist after wash. FIG. 4C: Mouse airway tissue were contracted with 1 μM methacholine for 5 min and then incubated overnight with 1 μM ISO±1 μM DFPQ. Airway slices were washed, re-contracted with methacholine, and incubated with ISO for 1 hour (n=4 bronchioles). Airway contractility was measured by luminal area of bronchioles by microscopy at various times. FIG. 4D: Bar graphs represent normalized ISO-promoted relaxation from methacholine contraction on day 2 of treatment at the 25 min timepoint from 4 tracheal rings. *, p<0.05 FIG. 4E: HASM cells were scratched and incubated overnight in PDGF containing serum free media. Cells were imaged to evaluate migration into the scratched area. FIG. 4F: Densitometric analysis of migration into scratched area (n=3 experiments). ***, p<0.001; **, p<0.01

FIGS. 5A-5E illustrate certain embodiments of the disclosure. FIG. 5A: Primary titration screening data of DFPQ. Data is expressed in triplicate as percent of response to 1 μM ISO in an enzyme complementarity assay for β-arrestin2 recruitment and cAMP sensor assay for cAMP concentration at the indicated compound concentration (log IC50_{Arr}=−6.3±0.1). FIG. 5B: β₂AR expressing HEK 293 cells were stimulated with 1 μM of the indicated agonists in the presence of 0.1% DMSO or 10 μM DFPQ for 10 min. cAMP production was measured by ELISA. FIGS. 5C and 5D: HEK 293 cells co-transfected with β-arrestin2-GFP10 and β₂AR-RLucII were pre-incubated with 10 μM DFPQ for 30 min. Cells were then incubated with Coelenterazine 400a for 2 min and then stimulated with 1 μM of the indicated β-agonist. Wells were read every 2 min post agonist addition. FIG. 5E: Lipid bicelles containing reconstituted β₂AR and Gₛ heterotrimer were pre-incubated with 0.1% DMSO or DFPQ and then stimulated with 1 μM ISO. Negative control samples did not contain ISO. Bound [³⁵S]-GTPγS was collected by rapid filtration on GF/B filters, washed 4 times with 4 ml of cold GTPγS wash buffer and analyzed by liquid scintillation.

### DETAILED DESCRIPTION

The present disclosure relates in part to the identification of novel β₂-adrenergic receptor-specific allosteric modulators. In certain embodiments, these compounds can be used to treat airway disease, such as but not limited to asthma and/or chronic obstructive pulmonary disease (COPD). In other non-limiting embodiments, these compounds selectively promote β₂AR interaction with Gₛ.

### Definitions

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, illustrative methods and materials are described. As used herein, each of the following terms has the meaning associated with it in this section.

Generally, the nomenclature used herein and the laboratory procedures in cell culture, molecular genetics, pharmacology, and organic chemistry are those well-known and commonly employed in the art.

Standard techniques are used for biochemical and/or biological manipulations. The techniques and procedures are generally performed according to conventional methods in the art and various general references (e.g., Sambrook and Russell, 2012, Molecular Cloning, A Laboratory Approach, Cold Spring Harbor Press, Cold Spring Harbor, NY, and Ausubel et al., 2002, Current Protocols in Molecular Biology, John Wiley & Sons, NY), which are provided throughout this document.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, “airway disease” means a restrictive respiratory condition such as asthma or COPD.

A disease or disorder is “alleviated” if the severity or frequency of at least one sign or symptom of the disease or disorder experienced by a patient is reduced.

As used herein, the terms “analog,” “analogue,” or “derivative” are meant to refer to a chemical compound or molecule made from a parent compound or molecule by one or more chemical reactions. As such, an analog can be a structure having a structure similar to that of the small molecule inhibitors described herein or can be based on a scaffold of a small molecule inhibitor described herein, but differing from it in respect to certain components or structural makeup, which may have a similar or opposite action metabolically.

As used herein, “asthma” means a chronic lung disease that inflames and narrows the airways. Asthma causes recurring periods of wheezing, chest tightness, shortness of breath, and coughing.

As used herein, “β2-adrenergic receptor” or “β₂AR” refers to the gene or protein product thereof having the amino acid sequence of SEQ ID NO:1, or a polymorphic variant thereof, for the human homolog.

As used herein, the term “binding” refers to the adherence of molecules to one another, such as, but not limited to, enzymes to substrates, antibodies to antigens, DNA strands to their complementary strands. Binding occurs because the shape and chemical nature of parts of the molecule surfaces are complementary. A common metaphor is the “lock-and-key” used to describe how enzymes fit around their substrate.

As used herein, “chronic obstructive pulmonary disease” or “COPD” means a chronic inflammatory lung disease that causes obstructed airflow from the lungs. Symptoms include breathing difficulty, cough, mucus production and wheezing.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” or “therapeutically effective amount” of a compound is that amount of compound sufficient to provide a beneficial effect to the subject to which the compound is administered. An “effective amount” of a delivery vehicle is that amount sufficient to effectively bind or deliver a compound.

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” or “therapeutically acceptable salt” refers to a salt of the administered compounds prepared from pharmaceutically acceptable non-toxic acids, including inorganic acids or bases, organic acids or bases, solvates (including hydrates), or clathrates thereof.

The terms “pharmaceutically effective amount” and “effective amount” refer to a nontoxic but sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation.

As used herein, the terms “polypeptide,” “protein,” and “peptide” are used interchangeably and refer to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

By the term “specifically binds,” as used herein, is meant a molecule, such as an antibody, which recognizes and binds to another molecule or feature, but does not substantially recognize or bind other molecules or features in a sample.

A “therapeutic” treatment is a treatment administered to a subject who exhibits signs of pathology, for the purpose of diminishing or eliminating those signs.

As used herein, the term “therapeutically effective amount” is an amount of a compound of the disclosure, that when administered to a patient, ameliorates a symptom of the disease or disorder. The amount of a compound of the disclosure that constitutes a “therapeutically effective amount” will vary depending on the compound, the disease state and its severity, the age of the patient to be treated, and the like. The therapeutically effective amount can be determined routinely by one of ordinary skill in the art having regard to his own knowledge and to this disclosure.

As used herein, the term “treating” or “treatment” of a state, disorder or condition includes: (i) inhibiting the state, disorder or condition, i.e., arresting or reducing the development of the disease or at least one clinical or subclinical symptom thereof, or (ii) relieving and/or ameliorating the disease, i.e. causing regression of the state, disorder or condition or at least one of its clinical or subclinical symptoms.

As used herein, the term “wild-type” refers to the genotype and phenotype that is characteristic of most of the members of a species occurring naturally and contrasting with the genotype and phenotype of a mutant.

As used herein, the term “alkyl,” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. Certain specific examples include (C₁-C₆)alkyl, such as, but not limited to, ethyl, methyl, isopropyl, isobutyl, n-pentyl, n-hexyl and cyclopropylmethyl.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. In certain embodiments, alkoxy includes (C₁-C₃)alkoxy, such as, but not limited to, ethoxy and methoxy.

As used herein, the term “aromatic” refers to a carbocycle or heterocycle with one or more polyunsaturated rings and having aromatic character, i.e. having (4n+2) delocalized (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. In certain embodiments, aryl includes phenyl and naphthyl, in particular, phenyl.

As used herein, the term “cycloalkyl” by itself or as part of another substituent refers to, unless otherwise stated, a cyclic chain hydrocarbon having the number of carbon atoms designated (i.e., C₃-C₆ refers to a cyclic group comprising a ring group consisting of three to six carbon atoms) and includes straight, branched chain or cyclic substituent groups. Examples of (C₃-C₆)cycloalkyl groups are cyclopropyl, cyclobutyl, cyclopentyl and cyclohexyl. Cycloalkyl rings can be optionally substituted. Non-limiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes bicyclic hydrocarbon rings, non-limiting examples of which include, bicyclo[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

As used herein, the term “substituted cycloalkyl” means cycloalkyl, as defined above, substituted by one, two or three substituents selected from the group consisting of halogen, —OH, alkoxy, alkyl, C═O, tetrahydro-2-H-pyranyl, —NH₂, —N(CH₂CH₃)₂, —N(CH₃)₂, (1-methyl-imidazol-2-yl), pyridin-2-yl, pyridin-3-yl, pyridin-4-yl, —C(═O)OH, trifluoromethyl, —C≡N, —C(═O)O(C₁-C₄)alkyl, —C(═O)NH₂, —C(═O)NH(C₁-C₄)alkyl, —C(═O)N((C₁-C₄)alkyl)₂, —SO₂NH₂, —C(═NH)NH₂, and —NO₂, advantageously containing one or two substituents selected from halogen, —OH, alkoxy, alkyl, C═O, —NH₂, trifluoromethyl, —N(CH₂CH₃)₂, and —C(═O)OH, more advantageously selected from halogen, C═O, alkyl, and N(CH₂CH₃)₂.

As used herein, the term “halide” refers to a halogen atom bearing a negative charge. The halide anions are fluoride (F⁻), chloride (Cl⁻), bromide (Br⁻), and iodide (I⁻).

As used herein, the term “halo” or “halogen” alone or as part of another substituent refers to, unless otherwise stated, a fluorine, chlorine, bromine, or iodine atom.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is a heteroaryl. In other embodiments, the heterocycle is a heterocycloalkyl.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

As used herein, the term “heterocycloalkyl” refers to a heterocycle that is non-aromatic in nature. The heterocycloalkyl may comprise a ring that is partially saturated.

Examples of non-aromatic heterocycles (“heterocycloalkyls”) include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (such as, but not limited to, 3-, 4-, 5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “substituted” means that an atom or group of atoms has replaced hydrogen as the substituent attached to another group.

For aryl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In other embodiments, the substituents vary in number between one and three. In yet another embodiments, the substituents vary in number between one and two. In yet another embodiments, the substituents are independently selected from the group consisting of C₁-6 alkyl, —OH, C₁-6 alkoxy, halo, amino, acetamido and nitro. As used herein, where a substituent is an alkyl or alkoxy group, the carbon chain may be branched, straight or cyclic, in particular, straight.

In certain embodiments, each occurrence of aryl, heteroaryl, or heterocycloalkyl is independently optionally substituted with at least one substituent selected from the group consisting of C₁-C₆ alkyl, C₃-C₈ cycloalkyl, phenyl, C₁-C₆ hydroxyalkyl, (C₁-C₆ alkoxy)-C₁-C₆ alkyl, C₁-C₆ haloalkyl, C₁-C₆ haloalkoxy, halogen, —CN, —OR^{b}, —N(R^{b})(R^{b}), —NO₂, —C(═O)N(R^{b})(R^{b}), —C(═O)OR^{b}, —OC(═O) R^{b}, —SR^{b}, —S(═O)R^{b}, —S(═O)₂R^{b}, N(R^{b})S(═O)₂R^{b}, —S(═O)₂N(R^{b})(R^{b}), acyl, and C₁-C₆ alkoxycarbonyl, wherein each occurrence of R b is independently H, C₁-C₆ alkyl, or C₃-C₈ cycloalkyl, wherein in R^{b} the alkyl or cycloalkyl is optionally substituted with at least one selected from the group consisting of halogen, —OH, C₁-C₆ alkoxy, and heteroaryl; or substituents on two adjacent carbon atoms combine to form —O(CH₂)₁₋₃O—.

In certain embodiments, each occurrence of alkyl, alkenyl, alkynyl, or cycloalkyl is independently optionally substituted with at least one substituent selected from the group consisting of C₁-C₆ alkyl, C₃-C₈ cycloalkyl, halo, cyano (—CN), —OR^{a}, optionally substituted phenyl, optionally substituted heteroaryl, optionally substituted heterocyclyl, —C(═O)OR^{a}, —OC(═O)R^{a}, —SR^{a}, —S(═O)R^{a}, —S(═O)₂R^{a}, —S(═O)₂NR^{a}R^{a}, —N(R^{a})S(═O)₂R^{a}, —N(R^{a})C(═O)R^{a}, —C(═O)NR^{a}R^{a}, and —N(R^{a})(R^{a}), wherein each occurrence of R^{a} is independently H, optionally substituted C₁-C₆ alkyl, optionally substituted C₃-C₈ cycloalkyl, optionally substituted aryl, or optionally substituted heteroaryl, or two R^{a} groups combine with the N to which they are bound to form a heterocycle.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Methods

The disclosure is based in part on the discovery that negative allosteric modulators of β₂AR are effective in the treatment of airway disease. Accordingly, in one aspect the disclosure provides a method of treating airway disease in a subject in need thereof. In certain embodiments, the method comprises administering to the subject a therapeutically effective amount of a compound of formula (1) or a pharmaceutically acceptable salt or solvate thereof. In other embodiments, the method comprises administering to the subject a therapeutically effective amount of a compound of formula (2) or a pharmaceutically acceptable salt or solvate thereof. In other embodiments, the method comprises administering to the subject a therapeutically effective amount of a compound of formula (3) or a pharmaceutically acceptable salt or solvate thereof. In yet another embodiment, the method comprises administering to the subject a compound of formula (1) or a pharmaceutically acceptable salt or solvate thereof and a compound of formula (2) or a pharmaceutically acceptable salt or solvate thereof. In yet another embodiment, the method comprises administering to the subject a compound of formula (1) or a pharmaceutically acceptable salt or solvate thereof and a compound of formula (3) or a pharmaceutically acceptable salt or solvate thereof. In yet another embodiment, the method comprises administering to the subject a compound of formula (2) or a pharmaceutically acceptable salt or solvate thereof and a compound of formula (3) or a pharmaceutically acceptable salt or solvate thereof. In certain embodiments, the airway disease is asthma and/or chronic obstructive pulmonary disease (COPD).

In certain embodiments, the subject is a mammal. In certain embodiments, the mammal is a human.

### Compounds and Compositions

In another aspect, the present disclosure relates to compounds which act as negative allosteric modulators of β₂AR. In some embodiments, the negative allosteric modulator of β₂AR is a compound of formula (1) or a salt, solvate, isotopologue, stereoisomer, tautomer, and/or any mixture thereof:

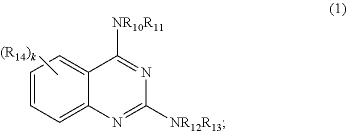

wherein:
R₁₀ and R₁₁ are each independently selected from the group consisting of H and optionally substituted C₃-C₈ cycloalkyl;
R₁₂ and R₁₃ are each independently selected from the group consisting of H; C₁-C₆ alkyl; C₁-C₆ alkoxy; optionally substituted C₃-C₈ cycloalkyl; —(CH₂)ₙO(CH₂)ₘCH₃; optionally monosubstituted phenyl wherein the optional substituent is selected from the group consisting of F, Br, —NO₂, CF₃, —S(═O)₂CH₃, —O(CH₂)ₚOCH₃, and

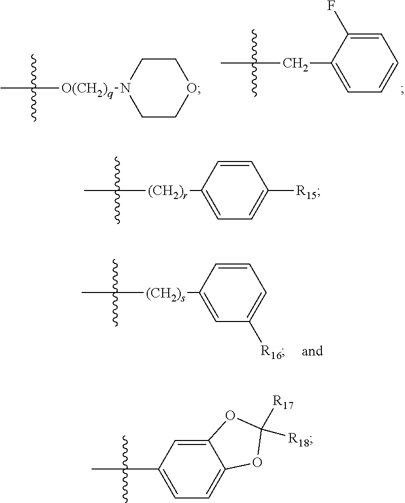

or

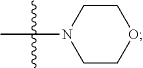 [SMILES: *N1CCOCC1]

R₁₂ and R₁₃ combine with the nitrogen to which they are attached to form each occurrence of R₁₄ is independently selected from the group consisting of H, C₁-C₆ alkyl, F, Br, Cl, and I;
R₁₅, R₁₇, and R₁₈ are each independently selected from the group consisting of C₁-C₆ alkyl, F, Br, Cl, and I;
R₁₆ is selected from the group consisting of F, Br, Cl, I, and —C(R₁₉)₃;
each R₁₉ is independently selected from the group consisting of F, Br, Cl, and I;
k is 4;
m, n, p, and q are each independently selected from the group consisting of 1, 2, and 3; and
r and s are each independently selected from the group consisting of 0 and 1.

In some embodiments, R₁₀ and R₁₁ are each H. In other embodiments, R₁₀ is H and R₁₁ is

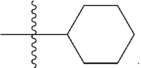 [SMILES: *C1CCCCC1]

In yet another embodiment, R₁₀ is H and R₁₁ is a C₆ cycloalkyl substituted with one or more of F, Br, Cl, or I. In some embodiments, R₁₀ is H and R₁₁ is

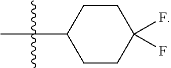 [SMILES: *C1CCC(F)(F)CC1]

In some embodiments, R₁₂ and R₁₃ are each H. In other embodiments, R₁₂ and R₁₃ are each C₁-C₆ alkyl. In some embodiments, R₁₂ and R₁₃ are each —CH₂CH₃. In other embodiments, R₁₂ is H and R₁₃ is a C₆ cycloalkyl. In some embodiments, R₁₂ is H and R₁₃ is a C₆ cycloalkyl substituted with C₁-C₆ alkyl. In certain embodiments, the C₃-C₈ cycloalkyl in one or more of R₁₂ and R₁₃ is substituted with at least one C₁-C₆ alkyl.

In some embodiments, R₁₂ is H and R₁₃ is

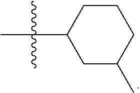 [SMILES: *C1CCCC(C)C1]

In other embodiments, R₁₂ is H and R₁₃ is unsubstituted phenyl. In other embodiments, R₁₂ is C₁-C₆ alkyl and R₁₃ is unsubstituted phenyl. In some embodiments, R₁₂ is —CH₃ and R₁₃ is unsubstituted phenyl. In other embodiments, R₁₂ is H and R₁₃ is

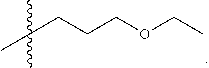 [SMILES: *CCCOCC]

In other embodiments, R₁₂ is H and R₁₃ is

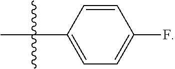 [SMILES: *c1ccc(F)cc1]

In other embodiments, R₁₂ is H and R₁₃ is

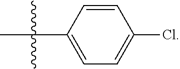 [SMILES: *c1ccc(Cl)cc1]

In other embodiments, R₁₂ is H and R₁₃ is

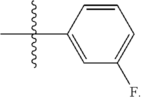 [SMILES: *c1cccc(F)c1]

In other embodiments, R₁₂ is H and R₁₃ is

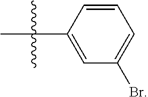 [SMILES: *c1cccc(Br)c1]

In other embodiments, R₁₂ is H and R₁₃ is

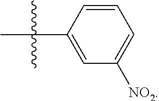 [SMILES: *c1cccc([N+](=O)[O-])c1]

In other embodiments, R₁₂ is H and R₁₃ is

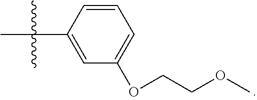 [SMILES: *c1cccc(OCCOC)c1]

In other embodiments, R₁₂ is H and R₁₃ is

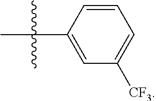 [SMILES: *c1cccc(C(F)(F)F)c1]

In other embodiments, R₁₂ is H and R₁₃ is

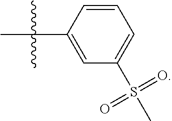 [SMILES: *c1cccc(S(C)(=O)=O)c1]

In other embodiments, R₁₂ is H and R₁₃ is

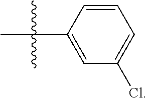 [SMILES: *c1cccc(Cl)c1]

In other embodiments, R₁₂ is H and R₁₃ is

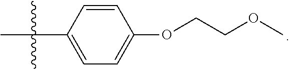 [SMILES: *c1ccc(OCCOC)cc1]

In other embodiments, R₁₂ is H and R₁₃ is

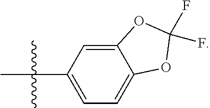 [SMILES: *c1ccc2c(c1)OC(F)(F)O2]

In other embodiments, R₁₂ is H and R₁₃ is

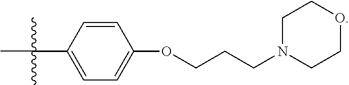 [SMILES: *c1ccc(OCCCN2CCOCC2)cc1]

In other embodiments, R₁₂ is H and R₁₃ is

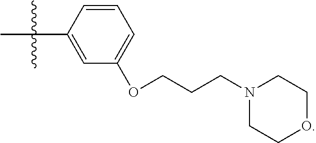 [SMILES: *c1cccc(OCCCN2CCOCC2)c1]

In other embodiments, R₁₂ is H and R₁₃ is

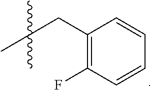 [SMILES: *Cc1ccccc1F]

In other embodiments, R₁₂ is H and R₁₃ is

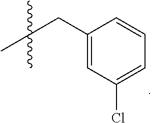 [SMILES: *Cc1cccc(Cl)c1]

In other embodiments, R₁₂ is H and R₁₃ is

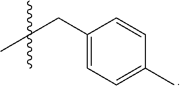 [SMILES: *Cc1ccc(C)cc1]

In some embodiments, R₁₃ is H and R₁₂ is

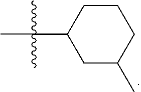 [SMILES: *C1CCCC(C)C1]

In other embodiments, R₁₃ is H and R₁₂ is unsubstituted phenyl. In other embodiments, R₁₃ is C₁-C₆ alkyl and R₁₂ is unsubstituted phenyl. In some embodiments, R₁₃ is —CH₃ and R₁₂ is unsubstituted phenyl. In other embodiments, R₁₃ is H and R₁₂ is

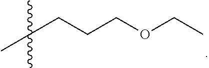 [SMILES: *CCCOCC]

In other embodiments, R₁₃ is H and R₁₂ is

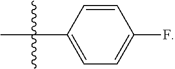 [SMILES: *c1ccc(F)cc1]

In other embodiments, R₁₃ is H and R₁₂ is

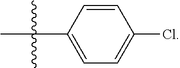 [SMILES: *c1ccc(Cl)cc1]

In other embodiments, R₁₃ is H and R₁₂ is

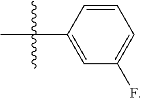 [SMILES: *c1cccc(F)c1]

In other embodiments, R₁₃ is H and R₁₂ is

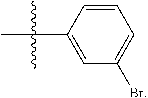 [SMILES: *c1cccc(Br)c1]

In other embodiments, R₁₃ is H and R₁₂ is

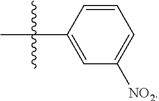 [SMILES: *c1cccc([N+](=O)[O-])c1]

In other embodiments, R₁₃ is H and R₁₂ is

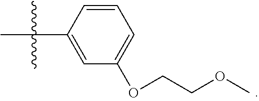 [SMILES: *c1cccc(OCCOC)c1]

In other embodiments, R₁₃ is H and R₁₂ is

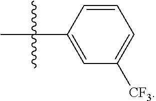 [SMILES: *c1cccc(C(F)(F)F)c1]

In other embodiments, R₁₃ is H and R₁₂ is

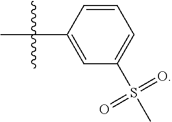 [SMILES: *c1cccc(S(C)(=O)=O)c1]

In other embodiments, R₁₃ is H and R₁₂ is

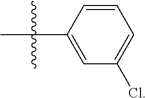 [SMILES: *c1cccc(Cl)c1]

In other embodiments, R₁₃ is H and R₁₂ is

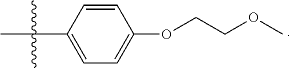 [SMILES: *c1ccc(OCCOC)cc1]

In other embodiments, R₁₃ is H and R₁₂ is

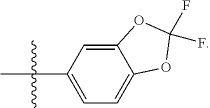 [SMILES: *c1ccc2c(c1)OC(F)(F)O2]

In other embodiments, R₁₃ is H and R₁₂ is

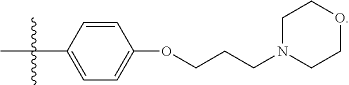 [SMILES: *c1ccc(OCCCN2CCOCC2)cc1]

In other embodiments, R₁₃ is H and R₁₂ is

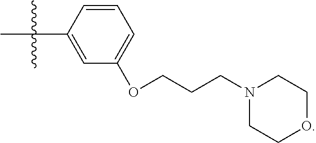 [SMILES: *c1cccc(OCCCN2CCOCC2)c1]

In other embodiments, R₁₃ is H and R₁₂ is

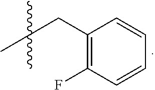 [SMILES: *Cc1ccccc1F]

In other embodiments, R₁₃ is H and R₁₂ is

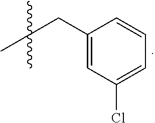 [SMILES: *Cc1cccc(Cl)c1]

In other embodiments, R₁₃ is H and R₁₂ is

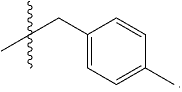 [SMILES: *Cc1ccc(C)cc1]

In yet another embodiment, R₁₂ and R₁₃ combine with the nitrogen to which they are attached to form

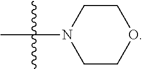 [SMILES: *N1CCOCC1]

In some embodiments, each R₁₄ is independently H. In other embodiments, one R₁₄ is Cl and three R₁₄ are H. In yet another embodiment, one R₁₄ is F and three are H.

In some embodiments, the compound of formula (1) is

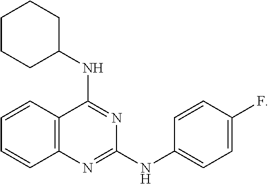 [SMILES: Fc1ccc(Nc2nc(NC3CCCCC3)c3ccccc3n2)cc1]

In some embodiments, the compound of formula (1) is

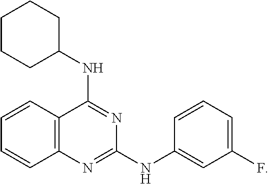 [SMILES: Fc1cccc(Nc2nc(NC3CCCCC3)c3ccccc3n2)c1]

In some embodiments, the compound of formula (1) is

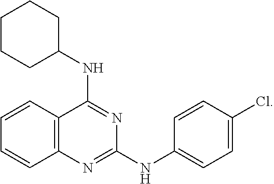 [SMILES: Clc1ccc(Nc2nc(NC3CCCCC3)c3ccccc3n2)cc1]

In some embodiments, the compound of formula (1) is

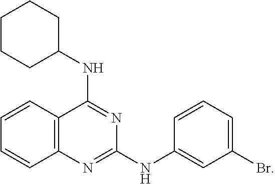 [SMILES: Brc1cccc(Nc2nc(NC3CCCCC3)c3ccccc3n2)c1]

In some embodiments, the compound of formula (1) is

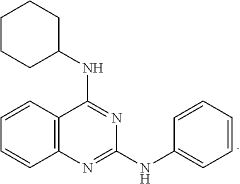 [SMILES: c1ccc(Nc2nc(NC3CCCCC3)c3ccccc3n2)cc1]

In some embodiments, the compound of formula (1) is

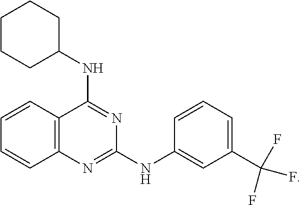 [SMILES: FC(F)(F)c1cccc(Nc2nc(NC3CCCCC3)c3ccccc3n2)c1]

In some embodiments, the compound of formula (1) is

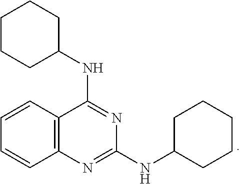 [SMILES: c1ccc2c(NC3CCCCC3)nc(NC3CCCCC3)nc2c1]

In some embodiments, the compound of formula (1) is

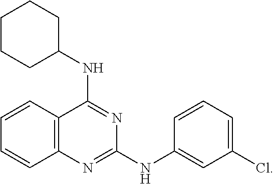 [SMILES: Clc1cccc(Nc2nc(NC3CCCCC3)c3ccccc3n2)c1]

In some embodiments, the compound of formula (1) is

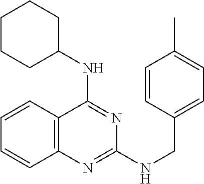 [SMILES: Cc1ccc(CNc2nc(NC3CCCCC3)c3ccccc3n2)cc1]

In some embodiments, the compound of formula (1) is

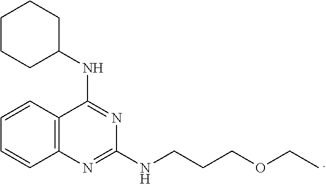 [SMILES: CCOCCCNc1nc(NC2CCCCC2)c2ccccc2n1]

In some embodiments, the compound of formula (1) is

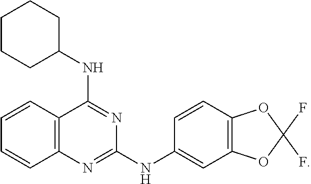 [SMILES: FC1(F)Oc2ccc(Nc3nc(NC4CCCCC4)c4ccccc4n3)cc2O1]

In some embodiments, the compound of formula (1) is

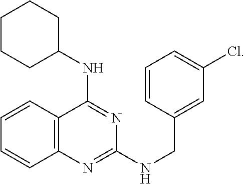 [SMILES: Clc1cccc(CNc2nc(NC3CCCCC3)c3ccccc3n2)c1]

In some embodiments, the compound of formula (1) is

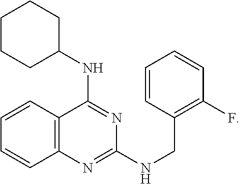 [SMILES: Fc1ccccc1CNc1nc(NC2CCCCC2)c2ccccc2n1]

In some embodiments, the compound of formula (1) is

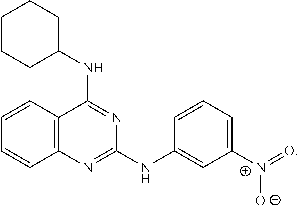 [SMILES: O=[N+]([O-])c1cccc(Nc2nc(NC3CCCCC3)c3ccccc3n2)c1]

In some embodiments, the compound of formula (1) is

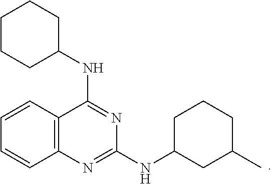 [SMILES: CC1CCCC(Nc2nc(NC3CCCCC3)c3ccccc3n2)C1]

In some embodiments, the compound of formula (1) is

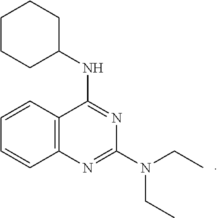 [SMILES: CCN(CC)c1nc(NC2CCCCC2)c2ccccc2n1]

In some embodiments, the compound of formula (1) is

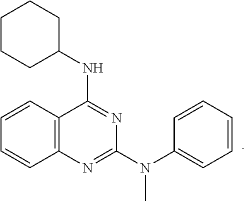 [SMILES: CN(c1ccccc1)c1nc(NC2CCCCC2)c2ccccc2n1]

In some embodiments, the compound of formula (1) is

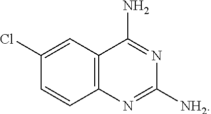 [SMILES: Nc1nc(N)c2cc(Cl)ccc2n1]

In some embodiments, the compound of formula (1) is

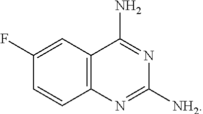 [SMILES: Nc1nc(N)c2cc(F)ccc2n1]

In some embodiments, the compound of formula (1) is

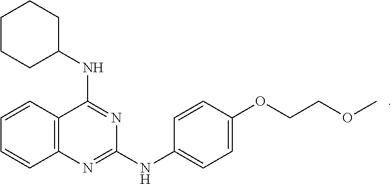 [SMILES: COCCOc1ccc(Nc2nc(NC3CCCCC3)c3ccccc3n2)cc1]

In some embodiments, the compound of formula (1) is

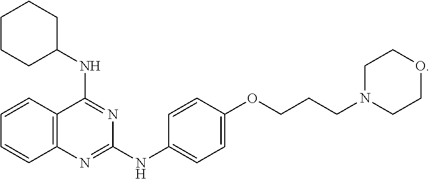 [SMILES: c1ccc2c(NC3CCCCC3)nc(Nc3ccc(OCCCN4CCOCC4)cc3)nc2c1]

In some embodiments, the compound of formula (1) is

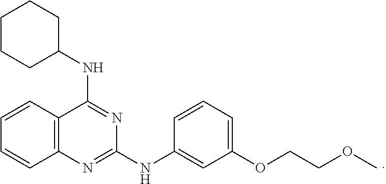 [SMILES: COCCOc1cccc(Nc2nc(NC3CCCCC3)c3ccccc3n2)c1]

In some embodiments, the compound of formula (1) is

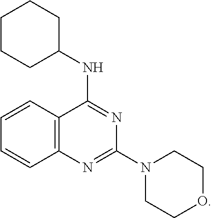 [SMILES: c1ccc2c(NC3CCCCC3)nc(N3CCOCC3)nc2c1]

In some embodiments, the compound of formula (1) is

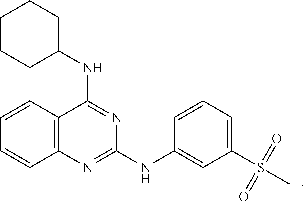 [SMILES: CS(=O)(=O)c1cccc(Nc2nc(NC3CCCCC3)c3ccccc3n2)c1]

In some embodiments, the compound of formula (1) is

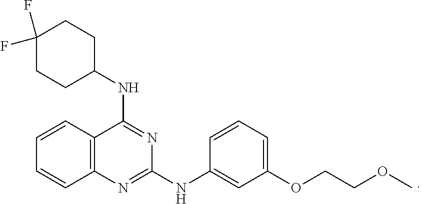 [SMILES: COCCOc1cccc(Nc2nc(NC3CCC(F)(F)CC3)c3ccccc3n2)c1]

In some embodiments, the compound of formula (1) is

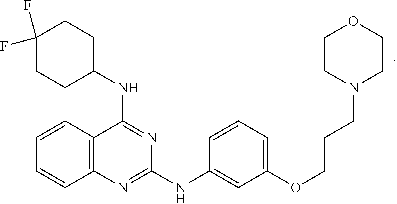 [SMILES: FC1(F)CCC(Nc2nc(Nc3cccc(OCCCN4CCOCC4)c3)nc3ccccc23)CC1]

In some embodiments, the compound of formula (1) or salt, solvate, isotopically labelled, stereoisomer, tautomer, and/or any mixture thereof is a compound of formula (1a), or a salt, solvate, isotopologue, stereoisomer, tautomer, and/or any mixture thereof:

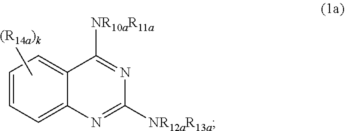

wherein:
R₁₀ₐ and R₁₁ₐ are each independently selected from the group consisting of H and optionally substituted C₃-C₈ cycloalkyl;
R₁₂ₐ is H;
R₁₃ₐ is a monosubstituted phenyl wherein the substituent is selected from —S(═O)₂CH₃, —(CH₂)ₙO(CH₂)ₘCH₃, and

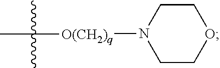

each occurrence of R₁₄ₐ is H;
k is 4; and
m and n are each independently 1, 2, or 3; with the proviso that, if R₁₃ₐ is phenyl monosubstituted with —(CH₂)ₙO(CH₂)ₘCH₃, then R₁₁ₐ is substituted C₃-C₈ cycloalkyl.

In some embodiments, R₁₀ₐ is H and R₁₁ₐ is

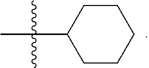 [SMILES: *C1CCCCC1]

In another embodiment, R₁₀ₐ is H and R₁₁ₐ is a C₆ cycloalkyl substituted with one or more of F, Br, Cl, or I. In some embodiments, R₁₀ₐ is H and R₁₁ₐ is

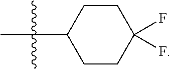 [SMILES: *C1CCC(F)(F)CC1]

In some embodiments, R₁₃ₐ is

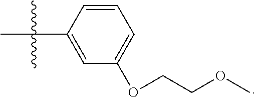 [SMILES: *c1cccc(OCCOC)c1]

In some embodiments, R₁₃ₐ is

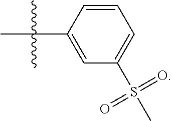 [SMILES: *c1cccc(S(C)(=O)=O)c1]

In some embodiments, R₁₃ₐ is

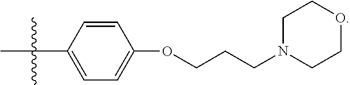 [SMILES: *c1ccc(OCCCN2CCOCC2)cc1]

In some embodiments, R₁₃ₐ is

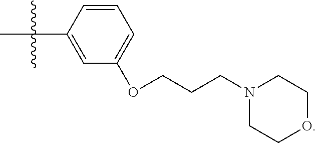 [SMILES: *c1cccc(OCCCN2CCOCC2)c1]

In some embodiments, when R₁₃ₐ is phenyl monosubstituted with —(CH₂)ₙO(CH₂)ₘCH₃, R₁₀ₐ is H and R₁₁ₐ is a substituted C₃-C₈ cycloalkyl. In one embodiment, when R₁₃ₐ is phenyl monosubstituted with —(CH₂)ₙO(CH₂)ₘCH₃, R₁₀ₐ is H, and R₁₁ₐ is a C₆ cycloalkyl substituted with F. In one embodiment, when R₁₃ₐ is phenyl monosubstituted with —(CH₂)ₙO(CH₂)ₘCH₃ and R₁₁ₐ is

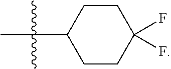 [SMILES: *C1CCC(F)(F)CC1]

In some embodiments, the compound of formula (1a) is

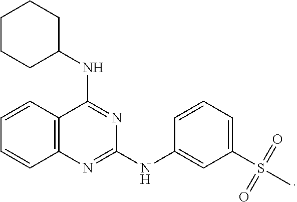 [SMILES: CS(=O)(=O)c1cccc(Nc2nc(NC3CCCCC3)c3ccccc3n2)c1]

In some embodiments, the compound of formula (1a) is

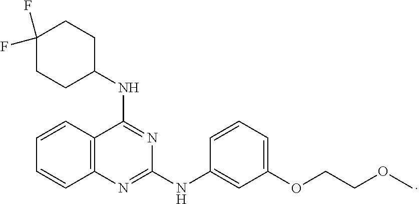 [SMILES: COCCOc1cccc(Nc2nc(NC3CCC(F)(F)CC3)c3ccccc3n2)c1]

In some embodiments, the compound of formula (1a) is

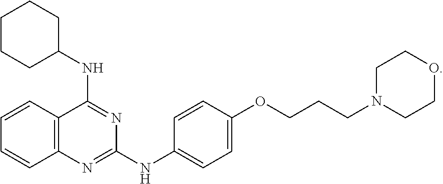 [SMILES: c1ccc2c(NC3CCCCC3)nc(Nc3ccc(OCCCN4CCOCC4)cc3)nc2c1]

In some embodiments, the compound of formula (1a) is

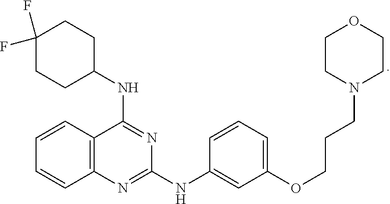 [SMILES: FC1(F)CCC(Nc2nc(Nc3cccc(OCCCN4CCOCC4)c3)nc3ccccc23)CC1]

In other embodiments, the negative allosteric modulator of β₂AR is a compound of formula (2) or a salt, solvate, isotopologue, stereoisomer, tautomer, and/or any mixture thereof:

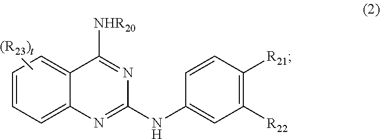

wherein:
R₂₀ is selected from the group consisting of C₃-C₈ cycloalkyl and C₄-C₇ heterocycloalkyl;
R₂₁ and R₂₂ are each independently selected from the group consisting of F, Cl, Br, I, C₁-C₆ alkoxy, and —SC(R₂₄)₃, with the proviso that R₂₁ is C₁-C₆ alkoxy if and only if R₂₂ is C₁-C₆ alkoxy;
each occurrence of R₂₃ is independently selected from the group consisting of H, C₁-C₆ alkyl, —C(R₂₅)₃, F, Cl, Br, and I;
each occurrence of R₂₄ and R₂₅ is independently selected from the group consisting of F, Cl, Br, and I; and
t is 4.

In some embodiments, R₂₀ is a C₆ cycloalkyl. In other embodiments, R₂₀ is a C₅ heterocycloalkyl comprising a nitrogen atom. In some embodiments, R₂₀ is

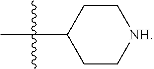 [SMILES: *C1CCNCC1]

In some embodiments, R₂₀ is

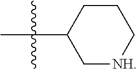 [SMILES: *C1CCCNC1]

In some embodiments, each of R₂₁ is F and R₂₂ is F. In other embodiments, each of R₂₁ is C₁ and R₂₂ is Cl. In other embodiments, each of R₂₁ is OCH₃ and R₂₂ is OCH₃. In other embodiments, R₂₁ is F and R₂₂ is Cl. In other embodiments, R₂₁ is C₁ and R₂₂ is F. In other embodiments, R₂₁ is C₁ and R₂₂ is SC(R₂₄)₃. In other embodiments, R₂₁ is SC(R₂₄)₃ and R₂₂ is Cl. In other embodiments, R₂₁ is C₁ and R₂₂ is SCF₃. In other embodiments, R₂₁ is SCF₃ and R₂₂ is Cl. In some embodiments, each of R₂₃ is H. In other embodiments, one of R₂₃ is F and three of R₂₃ are H. In other embodiments, one of R₂₃ is Cl and three of R₂₃ are H. In other embodiments, one of R₂₃ is Br and three of R₂₃ are H. In other embodiments, one of R₂₃ is C₁-C₆ alkyl and three of R₂₃ are H. In some embodiments, one of R₂₃ is CH₃ and three of R₂₃ are H. In yet another embodiment, one of R₂₃ is C(R₂₅)₃ and three of R₂₃ are H. In some embodiments, one of R₂₃ is —CF₃ and three of R₂₃ are H.

In some embodiments, the compound of formula (2) is

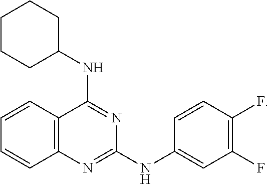 [SMILES: Fc1ccc(Nc2nc(NC3CCCCC3)c3ccccc3n2)cc1F]

In some embodiments, the compound of formula (2) is

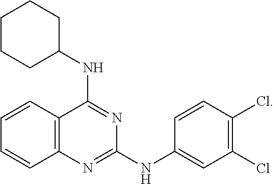 [SMILES: Clc1ccc(Nc2nc(NC3CCCCC3)c3ccccc3n2)cc1Cl]

In some embodiments, the compound of formula (2) is

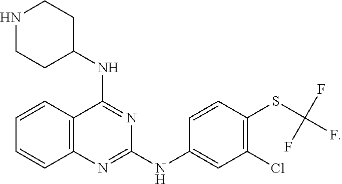 [SMILES: FC(F)(F)Sc1ccc(Nc2nc(NC3CCNCC3)c3ccccc3n2)cc1Cl]

In some embodiments, the compound of formula (2) is

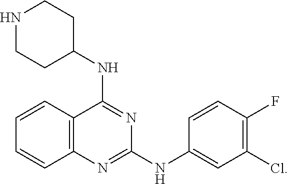 [SMILES: Fc1ccc(Nc2nc(NC3CCNCC3)c3ccccc3n2)cc1Cl]

In some embodiments, the compound of formula (2) is

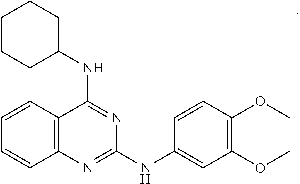 [SMILES: COc1ccc(Nc2nc(NC3CCCCC3)c3ccccc3n2)cc1OC]

In some embodiments, the compound of formula (2) is

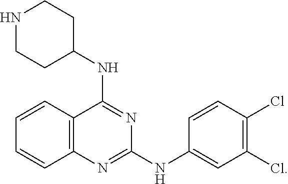 [SMILES: Clc1ccc(Nc2nc(NC3CCNCC3)c3ccccc3n2)cc1Cl]

In some embodiments, the compound of formula (2) is

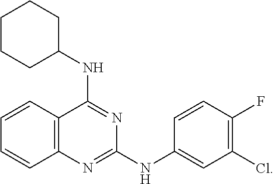 [SMILES: Fc1ccc(Nc2nc(NC3CCCCC3)c3ccccc3n2)cc1Cl]

In some embodiments, the compound of formula (2) is

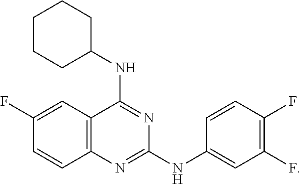 [SMILES: Fc1ccc2nc(Nc3ccc(F)c(F)c3)nc(NC3CCCCC3)c2c1]

In some embodiments, the compound of formula (2) is

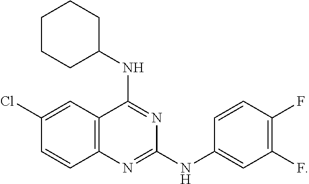 [SMILES: Fc1ccc(Nc2nc(NC3CCCCC3)c3cc(Cl)ccc3n2)cc1F]

In some embodiments, the compound of formula (2) is

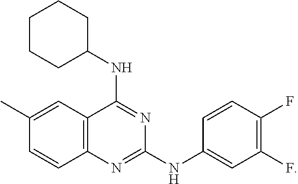 [SMILES: Cc1ccc2nc(Nc3ccc(F)c(F)c3)nc(NC3CCCCC3)c2c1]

In some embodiments, the compound of formula (2) is

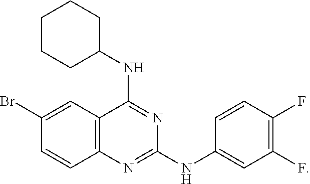 [SMILES: Fc1ccc(Nc2nc(NC3CCCCC3)c3cc(Br)ccc3n2)cc1F]

In some embodiments, the compound of formula (2) is

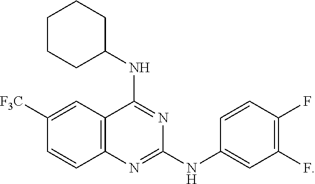 [SMILES: Fc1ccc(Nc2nc(NC3CCCCC3)c3cc(C(F)(F)F)ccc3n2)cc1F]

In some embodiments, the compound of formula (2) is

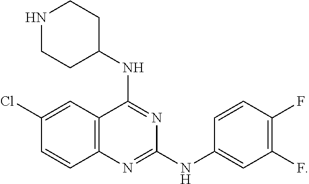 [SMILES: Fc1ccc(Nc2nc(NC3CCNCC3)c3cc(Cl)ccc3n2)cc1F]

In some embodiments, the compound of formula (2) is

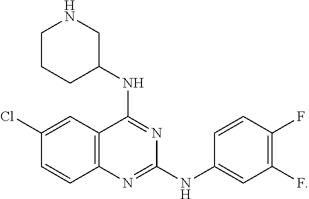 [SMILES: Fc1ccc(Nc2nc(NC3CCCNC3)c3cc(Cl)ccc3n2)cc1F]

In some embodiments, the compound of formula (2) or salt, solvate, isotopically labelled, stereoisomer, tautomer, and/or any mixture thereof is a compound of formula (2a) or a salt, solvate, isotopologue, stereoisomer, tautomer, and/or any mixture thereof:

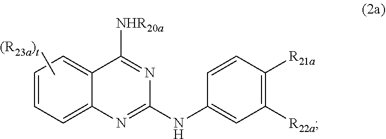

wherein:
R₂₀ₐ is selected from the group consisting of C₃-C₈ cycloalkyl and C₄-C₇ heterocycloalkyl;
R₂₁ₐ is F;
R₂₂ₐ is F;
each occurrence of R₂₃ₐ is independently selected from the group consisting of H, C₁-C₆ alkyl, —C(R₂₅ₐ)₃, F, Cl, Br, and I, wherein at least one R₂₃ₐ is selected from the group consisting of C₁-C₆ alkyl, —C(R₂₅ₐ)₃, F, Cl, Br, and I;
each R₂₅ₐ is independently selected from the group consisting of F, Cl, Br, and I; and
t is 4.

In some embodiments, R₂₀ₐ is a C₆ cycloalkyl. In certain embodiments, R₂₀ₐ is cyclohexyl. In other embodiments, R₂₀ₐ is a C₅ heterocycloalkyl comprising a nitrogen atom. In some embodiments, R₂₀ₐ is

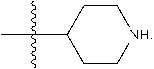 [SMILES: *C1CCNCC1]

In some embodiments, R₂₀ₐ is

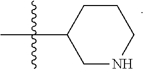 [SMILES: *C1CCCNC1]

In some embodiments, one of R₂₃ₐ is selected from F, Cl, or Br and three of R₂₃ₐ are H. In other embodiments, one of R₂₃ₐ is C₁-C₆ alkyl and three of R₂₃ are H. In some embodiments, one of R₂₃ₐ is —CH₃ and three of R₂₃ₐ are H. In yet another embodiment, one of R₂₃ₐ is —C(R₂₅ₐ)₃ and three of R₂₃ₐ are H. In some embodiments, one of R₂₃ₐ is —CF₃ and three of R₂₃ₐ are H.

In some embodiments, the compound of formula (2a) is

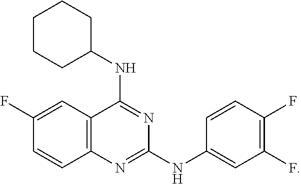 [SMILES: Fc1ccc2nc(Nc3ccc(F)c(F)c3)nc(NC3CCCCC3)c2c1]

In some embodiments, the compound of formula (2a) is

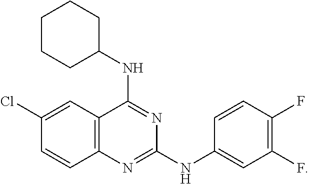 [SMILES: Fc1ccc(Nc2nc(NC3CCCCC3)c3cc(Cl)ccc3n2)cc1F]

In some embodiments, the compound of formula (2a) is

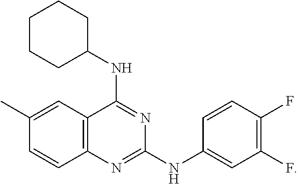 [SMILES: Cc1ccc2nc(Nc3ccc(F)c(F)c3)nc(NC3CCCCC3)c2c1]

In some embodiments, the compound of formula (2a) is

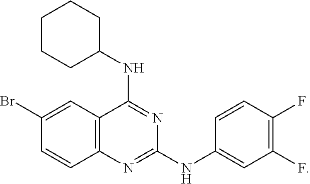 [SMILES: Fc1ccc(Nc2nc(NC3CCCCC3)c3cc(Br)ccc3n2)cc1F]

In some embodiments, the compound of formula (2a) is

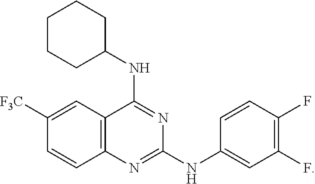 [SMILES: Fc1ccc(Nc2nc(NC3CCCCC3)c3cc(C(F)(F)F)ccc3n2)cc1F]

In some embodiments, the compound of formula (2a) is

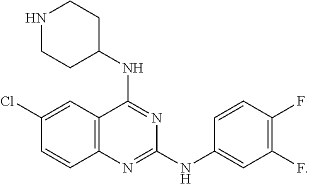 [SMILES: Fc1ccc(Nc2nc(NC3CCNCC3)c3cc(Cl)ccc3n2)cc1F]

In some embodiments, the compound of formula (2a) is

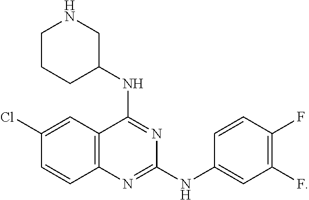 [SMILES: Fc1ccc(Nc2nc(NC3CCCNC3)c3cc(Cl)ccc3n2)cc1F]

In other embodiments, the negative allosteric modulator of β₂AR is a compound of formula (3) or a salt, solvate, isotopologue, stereoisomer, tautomer, and/or any mixture thereof:

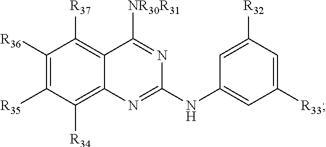

wherein:
R₃₀ and R₃₁ are each independently selected from the group consisting of: H; C₁-C₆ linear alkyl optionally substituted with at least one substituent selected from the group consisting of NR₃₈R₃₉, C₃-C₈ cycloalkyl, phenyl, and OH; C₃-C₁₂ cycloalkyl optionally substituted with at least one substituent selected from the group consisting of C₁-C₆ alkyl, C═O, and NR₃₈R₃₉; optionally substituted C₄-C₇ heterocycloalkyl optionally substituted with at least one substituent selected from the group consisting of phenyl, benzyl, CH₂CH₂OCH₃, and C(═O)R₄₁; phenyl substituted with one or more substituents selected from the group consisting of F, Br, I, OH, C₁-C₆ alkyl, C₁-C₆ alkoxy, N(R₄₀)₂, and C(═O)R₄₁;

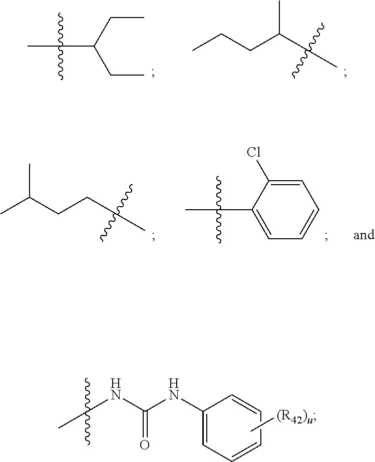

R₃₂ and R₃₃ are each independently selected from the group consisting of F, Cl, Br, and I;
R₃₄ and R₃₆ are each independently selected from the group consisting of H, C₁-C₆ alkyl, C₁-C₆ alkoxy, F, Cl, Br, and I;
R₃₅ and R₃₇ are each independently selected from the group consisting of H and C₁-C₆ alkyl;
R₃₈ and R₃₉ are each independently selected from the group consisting of H, C₁-C₆ alkyl, and C₆-C₁₂ aryl;
each occurrence R₄₀ is selected from the group consisting of H and C₁-C₆ linear alkyl;
R₄₁ is selected from the group consisting of H, C₁-C₆ alkyl, and C₁-C₆ alkoxy;
R₄₂ is selected from the group consisting of F, Cl, Br, and I; and
u is selected from the group consisting of 1, 2, 3, 4, and 5.

In some embodiments, R₃₀ is H and R₃₁ is H. In other embodiments, R₃₀ is C₁-C₆ linear alkyl and R₃₁ is C₁-C₆ linear alkyl. In some embodiments, R₃₀ is CH₂CH₃ and R₃₁ is CH₂CH₃. In other embodiments, R₃₀ is H and R₃₁ is an unsubstituted C₁-C₆ linear alkyl. In some embodiments, R₃₀ is H and R₃₁ is selected from CH₂CH₃ and —(CH₂)₄CH₃. In other embodiments, R₃₀ is H and R₃₁ is a substituted C₁-C₆ linear alkyl. In some embodiments, R₃₀ is H and R₃₁ is a substituted C₁-C₆ linear alkyl wherein the substituent is selected from OH, phenyl, C₆ cycloalkyl, NH₂, N(CH₃)₂, and NH(C₆-C₁₂ aryl). In some embodiments, R₃₀ is H and R₃₁ is selected from —(CH₂)₂₀H, —(CH₂)phenyl, —(CH₂)₂phenyl, —(CH₂)cyclohexane, —(CH₂)₂cyclohexane, —(CH₂)₃NH₂, —(CH₂)₃N(CH₃)₂, and

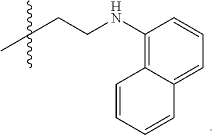 [SMILES: *CCNc1cccc2ccccc12]

In other embodiments, R₃₀ is H and R₃₁ is an unsubstituted C₃-C₁₂ cycloalkyl. In some embodiments, R₃₀ is H and R₃₁ is selected from cyclopentane, cyclohexane, cycloheptane, and adamantyl. In other embodiments, R₃₀ is H and R₃₁ is a substituted C₃-C₁₂ cycloalkyl. In some embodiments, R₃₀ is H and R₃₁ is a substituted C₆ cycloalkyl wherein the substituent is selected from —CH₃, C═O, and —N(CH₂CH₃)₂. In some embodiments, R₃₀ is H and R₃₁ is

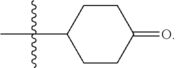 [SMILES: *C1CCC(=O)CC1]

In some embodiments, R₃₀ is H and R₃₁ is

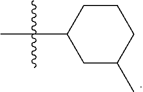 [SMILES: *C1CCCC(C)C1]

In some embodiments, R₃₀ is H and R₃₁ is

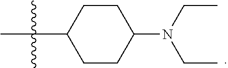 [SMILES: *C1CCC(N(CC)CC)CC1]

In other embodiments, R₃₀ is H and R₃₁ is an unsubstituted C₄-C₇ heterocycloalkyl. In some embodiments, R₃₀ is H and R₃₁ is an unsubstituted C₅ heterocycloalkyl comprising one nitrogen atom. In some embodiments, R₃₀ is H and R₃₁ is unsubstituted piperidine. In other embodiments, R₃₀ is H and R₃₁ is a substituted C₄-C₇ heterocycloalkyl. In some embodiments, R₃₀ is H and R₃₁ is a substituted C₅ heterocycloalkyl comprising one nitrogen atom. In some embodiments, R₃₀ is H and R₃₁ is substituted piperidine wherein the substituent is selected from phenyl, —(C═O)O(C₁-C₆ alkyl), —(C₁-C₆ alkyl)OCH₃, and —(C₁-C₆ alkyl)phenyl. In some embodiments, R₃₀ is H and R₃₁ is

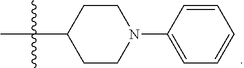 [SMILES: *C1CCN(c2ccccc2)CC1]

In some embodiments, R₃₀ is H and R₃₁ is

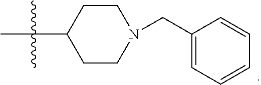 [SMILES: *C1CCN(Cc2ccccc2)CC1]

In some embodiments, R₃₀ is H and R₃₁ is

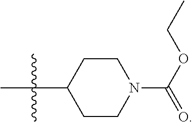 [SMILES: *C1CCN(C(=O)OCC)CC1]

In some embodiments, R₃₀ is H and R₃₁ is

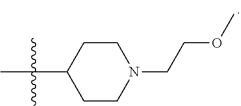 [SMILES: *C1CCN(CCOC)CC1]

In other embodiments, R₃₀ is H and R₃₁ is substituted phenyl, wherein the substituent is selected from F, —(CH₂)₂₀H, —OCH₃, —C(═O)CH₃, —N(CH₂CH₃)₂, and combinations thereof. In some embodiments, R₃₀ is H and R₃₁ is

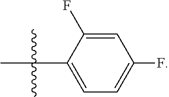 [SMILES: *c1ccc(F)cc1F]

In some embodiments, R₃₀ is H and R₃₁ is

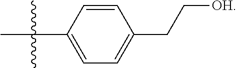 [SMILES: *c1ccc(CCO)cc1]

In some embodiments, R₃₀ is H and R₃₁ is

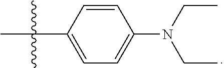 [SMILES: *c1ccc(N(CC)CC)cc1]

In some embodiments, R₃₀ is H and R₃₁ is

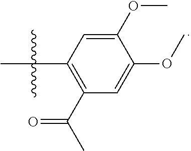 [SMILES: *c1cc(OC)c(OC)cc1C(C)=O]

In other embodiments, R₃₀ is H and R₃₁ is

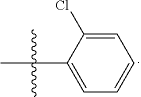 [SMILES: *c1ccccc1Cl]

In other embodiments, R₃₀ is H and R₃₁ is

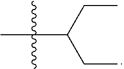 [SMILES: *C(CC)CC]

In other embodiments, R₃₀ is H and R₃₁

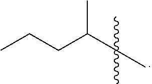 [SMILES: *C(C)CCC]

In other embodiments, R₃₀ is H and R₃₁

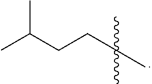 [SMILES: *CCC(C)C]

In yet another embodiment, R₃₀ is H and R₃₁ is

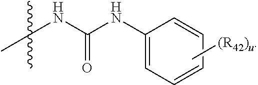

In some embodiments, R₃₀ is H and R₃₁ is

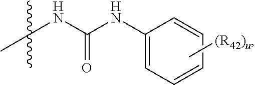

wherein R₄₂ is F and u is 1.

In some embodiments, R₃₂ is Cl and R₃₃ is Cl. In other embodiments, R₃₂ is F and R₃₃ is F. In other embodiments, R₃₂ is F and R₃₃ is I.

In some embodiments, R₃₄ is H. In other embodiments, R₃₄ is C₁-C₆ alkyl. In some embodiments, R₃₄ is CH₃.

In some embodiments, R₃₅ is H. In other embodiments, R₃₅ is C₁-C₆ alkyl. In some embodiments, R₃₅ is CH₃.

In some embodiments, R₃₆ is H. In other embodiments, R₃₆ is C₁-C₆ alkyl. In some embodiments, R₃₆ is CH₃. In some embodiments, R₃₆ is F. In some embodiments, R₃₆ is Cl.

In some embodiments, R₃₇ is H.

In some embodiments, the compound of formula (3) is

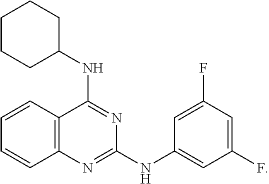 [SMILES: Fc1cc(F)cc(Nc2nc(NC3CCCCC3)c3ccccc3n2)c1]

In some embodiments, the compound of formula (3) is

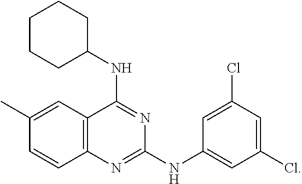 [SMILES: Cc1ccc2nc(Nc3cc(Cl)cc(Cl)c3)nc(NC3CCCCC3)c2c1]

In some embodiments, the compound of formula (3) is

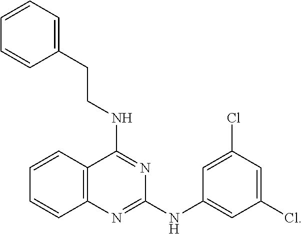 [SMILES: Clc1cc(Cl)cc(Nc2nc(NCCc3ccccc3)c3ccccc3n2)c1]

In some embodiments, the compound of formula (3) is

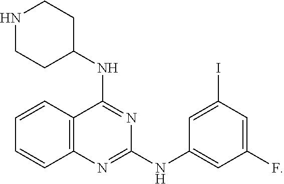 [SMILES: Fc1cc(I)cc(Nc2nc(NC3CCNCC3)c3ccccc3n2)c1]

In some embodiments, the compound of formula (3) is

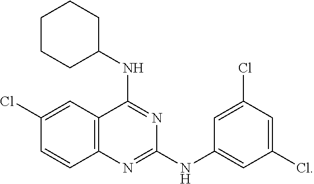 [SMILES: Clc1cc(Cl)cc(Nc2nc(NC3CCCCC3)c3cc(Cl)ccc3n2)c1]

In some embodiments, the compound of formula (3) is

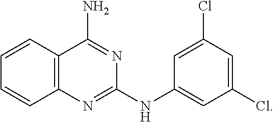 [SMILES: Nc1nc(Nc2cc(Cl)cc(Cl)c2)nc2ccccc12]

In some embodiments, the compound of formula (3) is

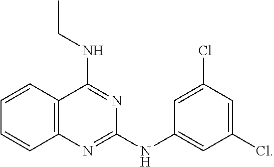 [SMILES: CCNc1nc(Nc2cc(Cl)cc(Cl)c2)nc2ccccc12]

In some embodiments, the compound of formula (3) is

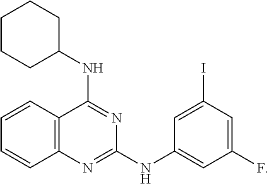 [SMILES: Fc1cc(I)cc(Nc2nc(NC3CCCCC3)c3ccccc3n2)c1]

In some embodiments, the compound of formula (3) is

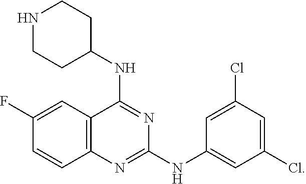 [SMILES: Fc1ccc2nc(Nc3cc(Cl)cc(Cl)c3)nc(NC3CCNCC3)c2c1]

In some embodiments, the compound of formula (3) is

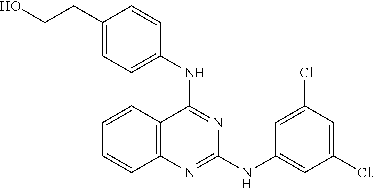 [SMILES: OCCc1ccc(Nc2nc(Nc3cc(Cl)cc(Cl)c3)nc3ccccc23)cc1]

In some embodiments, the compound of formula (3) is

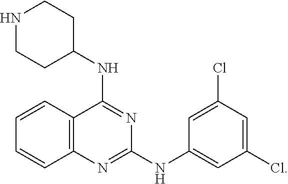 [SMILES: Clc1cc(Cl)cc(Nc2nc(NC3CCNCC3)c3ccccc3n2)c1]

In some embodiments, the compound of formula (3) is

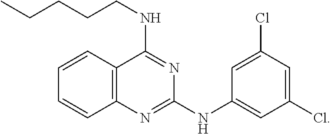 [SMILES: CCCCCNc1nc(Nc2cc(Cl)cc(Cl)c2)nc2ccccc12]

In some embodiments, the compound of formula (3) is

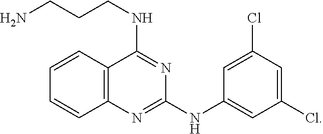 [SMILES: NCCCNc1nc(Nc2cc(Cl)cc(Cl)c2)nc2ccccc12]

In some embodiments, the compound of formula (3) is

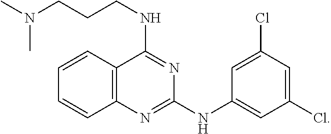 [SMILES: CN(C)CCCNc1nc(Nc2cc(Cl)cc(Cl)c2)nc2ccccc12]

In some embodiments, the compound of formula (3) is

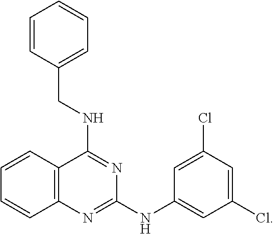 [SMILES: Clc1cc(Cl)cc(Nc2nc(NCc3ccccc3)c3ccccc3n2)c1]

In some embodiments, the compound of formula (3) is

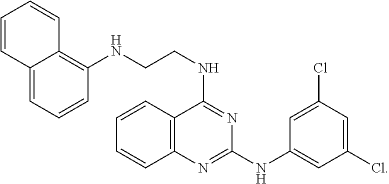 [SMILES: Clc1cc(Cl)cc(Nc2nc(NCCNc3cccc4ccccc34)c3ccccc3n2)c1]

In some embodiments, the compound of formula (3) is

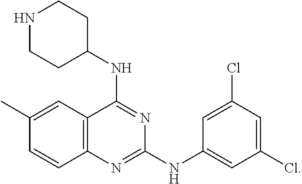 [SMILES: Cc1ccc2nc(Nc3cc(Cl)cc(Cl)c3)nc(NC3CCNCC3)c2c1]

In some embodiments, the compound of formula (3) is

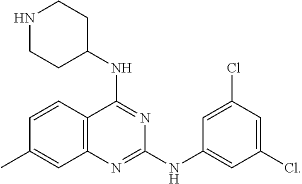 [SMILES: Cc1ccc2c(NC3CCNCC3)nc(Nc3cc(Cl)cc(Cl)c3)nc2c1]

In some embodiments, the compound of formula (3) is

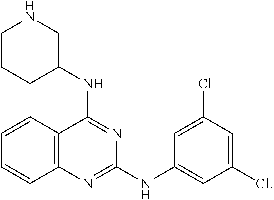 [SMILES: Clc1cc(Cl)cc(Nc2nc(NC3CCCNC3)c3ccccc3n2)c1]

In some embodiments, the compound of formula (3) is

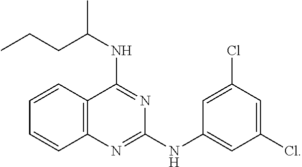 [SMILES: CCCC(C)Nc1nc(Nc2cc(Cl)cc(Cl)c2)nc2ccccc12]

In some embodiments, the compound of formula (3) is

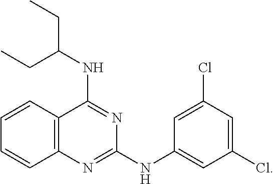 [SMILES: CCC(CC)Nc1nc(Nc2cc(Cl)cc(Cl)c2)nc2ccccc12]

In some embodiments, the compound of formula (3) is

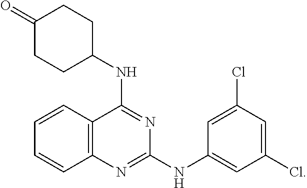 [SMILES: O=C1CCC(Nc2nc(Nc3cc(Cl)cc(Cl)c3)nc3ccccc23)CC1]

In some embodiments, the compound of formula (3) is

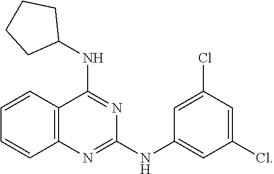 [SMILES: Clc1cc(Cl)cc(Nc2nc(NC3CCCC3)c3ccccc3n2)c1]

In some embodiments, the compound of formula (3) is

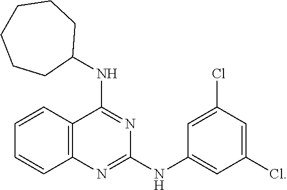 [SMILES: Clc1cc(Cl)cc(Nc2nc(NC3CCCCCC3)c3ccccc3n2)c1]

In some embodiments, the compound of formula (3) is

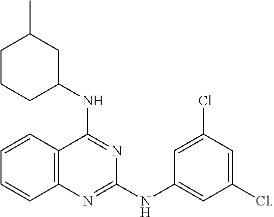 [SMILES: CC1CCCC(Nc2nc(Nc3cc(Cl)cc(Cl)c3)nc3ccccc23)C1]

In some embodiments, the compound of formula (3) is

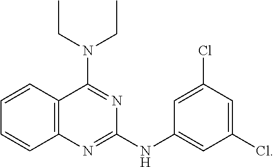 [SMILES: CCN(CC)c1nc(Nc2cc(Cl)cc(Cl)c2)nc2ccccc12]

In some embodiments, the compound of formula (3) is

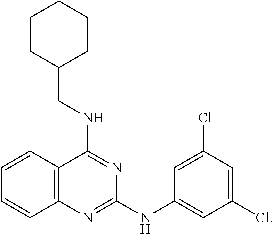 [SMILES: Clc1cc(Cl)cc(Nc2nc(NCC3CCCCC3)c3ccccc3n2)c1]

In some embodiments, the compound of formula (3) is

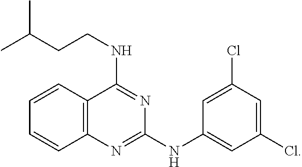 [SMILES: CC(C)CCNc1nc(Nc2cc(Cl)cc(Cl)c2)nc2ccccc12]

In some embodiments, the compound of formula (3) is

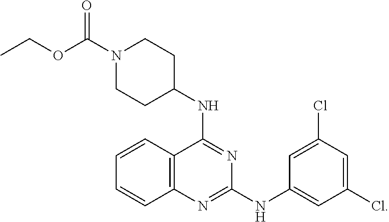 [SMILES: CCOC(=O)N1CCC(Nc2nc(Nc3cc(Cl)cc(Cl)c3)nc3ccccc23)CC1]

In some embodiments, the compound of formula (3) is

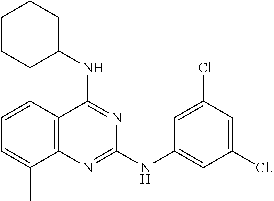 [SMILES: Cc1cccc2c(NC3CCCCC3)nc(Nc3cc(Cl)cc(Cl)c3)nc12]

In some embodiments, the compound of formula (3) is

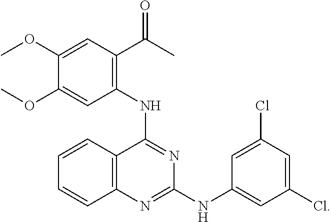 [SMILES: COc1cc(Nc2nc(Nc3cc(Cl)cc(Cl)c3)nc3ccccc23)c(C(C)=O)cc1OC]

In some embodiments, the compound of formula (3) is

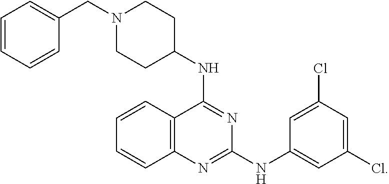 [SMILES: Clc1cc(Cl)cc(Nc2nc(NC3CCN(Cc4ccccc4)CC3)c3ccccc3n2)c1]

In some embodiments, the compound of formula (3) is

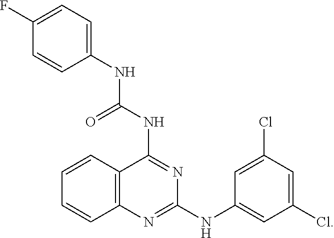 [SMILES: O=C(Nc1ccc(F)cc1)Nc1nc(Nc2cc(Cl)cc(Cl)c2)nc2ccccc12]

In some embodiments, the compound of formula (3) is

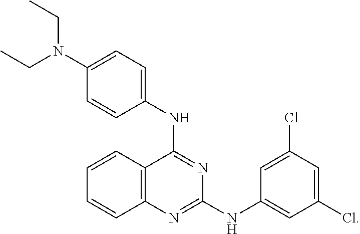 [SMILES: CCN(CC)c1ccc(Nc2nc(Nc3cc(Cl)cc(Cl)c3)nc3ccccc23)cc1]

In some embodiments, the compound of formula (3) is

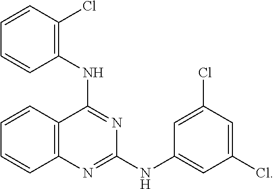 [SMILES: Clc1cc(Cl)cc(Nc2nc(Nc3ccccc3Cl)c3ccccc3n2)c1]

In some embodiments, the compound of formula (3) is

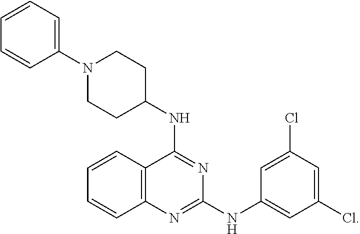 [SMILES: Clc1cc(Cl)cc(Nc2nc(NC3CCN(c4ccccc4)CC3)c3ccccc3n2)c1]

In some embodiments, the compound of formula (3) is

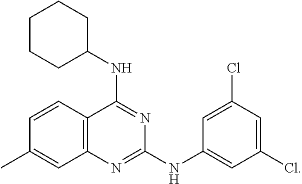 [SMILES: Cc1ccc2c(NC3CCCCC3)nc(Nc3cc(Cl)cc(Cl)c3)nc2c1]

### In

some embodiments, the compound of formula (3) is

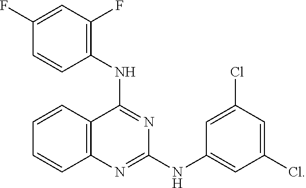 [SMILES: Fc1ccc(Nc2nc(Nc3cc(Cl)cc(Cl)c3)nc3ccccc23)c(F)c1]

In some embodiments, the compound of formula (3) is

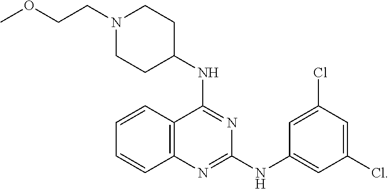 [SMILES: COCCN1CCC(Nc2nc(Nc3cc(Cl)cc(Cl)c3)nc3ccccc23)CC1]

In some embodiments, the compound of formula (3) is

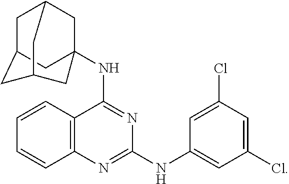 [SMILES: Clc1cc(Cl)cc(Nc2nc(NC34CC5CC(CC(C5)C3)C4)c3ccccc3n2)c1]

In some embodiments, the compound of formula (3) is

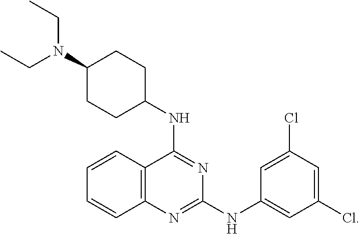

In some embodiments, the compound of formula (3) is

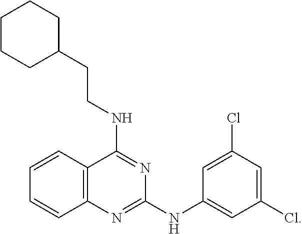 [SMILES: Clc1cc(Cl)cc(Nc2nc(NCCC3CCCCC3)c3ccccc3n2)c1]

TABLE 1

| Cmpd | Structure | Name |
|---|---|---|
| 1 | 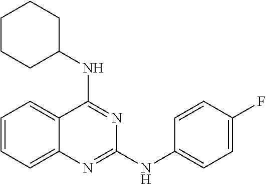 [SMILES: Fc1ccc(Nc2nc(NC3CCCCC3)c3ccccc3n2)cc1] | N4-cyclohexyl-N2-(4- fluorophenyl)quinazoline-2,4-diamine |
| 2 | 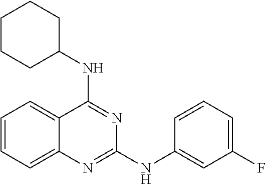 [SMILES: Fc1cccc(Nc2nc(NC3CCCCC3)c3ccccc3n2)c1] | N4-cyclohexyl-N2-(3- fluorophenyl)quinazoline-2,4-diamine |
| 3 | 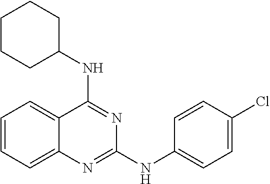 [SMILES: Clc1ccc(Nc2nc(NC3CCCCC3)c3ccccc3n2)cc1] | N2-(4-chlorophenyl)-N4- cyclohexylquinazoline-2,4-diamine |
| 4 | 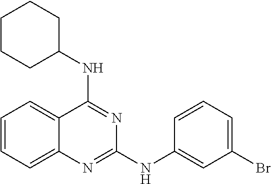 [SMILES: Brc1cccc(Nc2nc(NC3CCCCC3)c3ccccc3n2)c1] | N2-(3-bromophenyl)-N4- cyclohexylquinazoline-2,4-diamine |
| 5 | 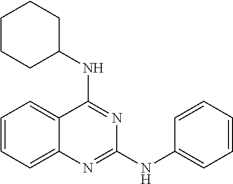 [SMILES: c1ccc(Nc2nc(NC3CCCCC3)c3ccccc3n2)cc1] | N4-cyclohexyl-N2-phenylquinazoline-2,4- diamine |
| 6 | 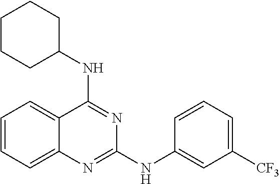 [SMILES: FC(F)(F)c1cccc(Nc2nc(NC3CCCCC3)c3ccccc3n2)c1] | N4-cyclohexyl-N2-(3- (trifluoromethyl)phenyl)quinazoline-2,4- diamine |
| 7 | 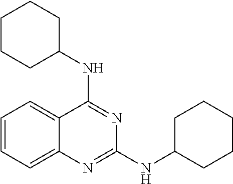 [SMILES: c1ccc2c(NC3CCCCC3)nc(NC3CCCCC3)nc2c1] | N2,N4-dicyclohexylquinazoline-2,4-diamine |
| 8 | 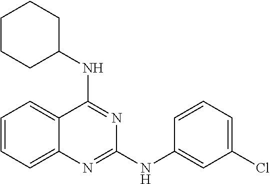 [SMILES: Clc1cccc(Nc2nc(NC3CCCCC3)c3ccccc3n2)c1] | N2-(3-chlorophenyl)-N4- cyclohexylquinazoline-2,4-diamine |
| 9 | 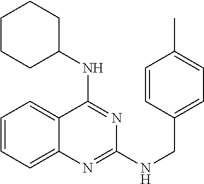 [SMILES: Cc1ccc(CNc2nc(NC3CCCCC3)c3ccccc3n2)cc1] | N4-cyclohexyl-N2-(4- methylbenzyl)quinazoline-2,4-diamine |
| 10 | 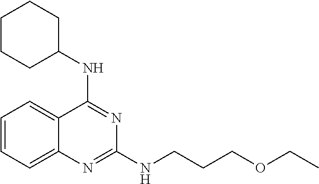 [SMILES: CCOCCCNc1nc(NC2CCCCC2)c2ccccc2n1] | N4-cyclohexyl-N2-(3- ethoxypropyl)quinazoline-2,4-diamine |
| 11 | 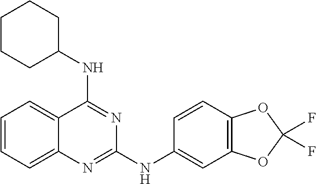 [SMILES: FC1(F)Oc2ccc(Nc3nc(NC4CCCCC4)c4ccccc4n3)cc2O1] | N4-cyclohexyl-N2-(2,2- difluorobenzo[d][1,3]dioxol-5- yl)quinazoline-2,4-diamine |
| 12 | 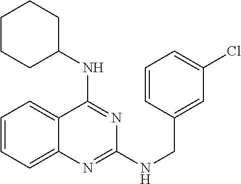 [SMILES: Clc1cccc(CNc2nc(NC3CCCCC3)c3ccccc3n2)c1] | N2-(3-chlorobenzyl)-N4- cyclohexylquinazoline-2,4-diamine |
| 13 | 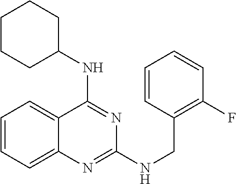 [SMILES: Fc1ccccc1CNc1nc(NC2CCCCC2)c2ccccc2n1] | N4-cyclohexyl-N2-(2- fluorobenzyl)quinazoline-2,4-diamine |
| 14 | 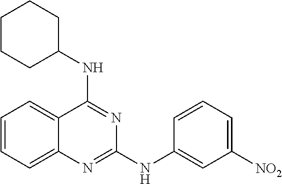 [SMILES: O=[N+]([O-])c1cccc(Nc2nc(NC3CCCCC3)c3ccccc3n2)c1] | N4-cyclohexyl-N2-(3- nitrophenyl)quinazoline-2,4-diamine |
| 15 | 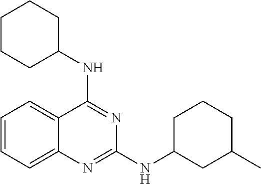 [SMILES: CC1CCCC(Nc2nc(NC3CCCCC3)c3ccccc3n2)C1] | N4-cyclohexyl-N2-(3- methylcyclohexyl)quinazoline-2,4-diamine |
| 16 | 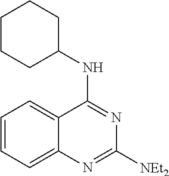 | N4-cyclohexyl-N2,N2-diethylquinazoline- 2,4-diamine |
| 17 | 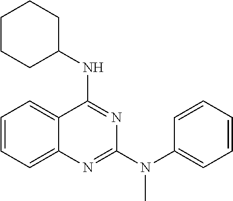 [SMILES: CN(c1ccccc1)c1nc(NC2CCCCC2)c2ccccc2n1] | N4-cyclohexyl-N2-methyl-N2- phenylquinazoline-2,4-diamine |
| 18 | 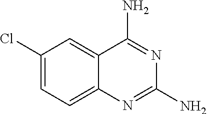 [SMILES: Nc1nc(N)c2cc(Cl)ccc2n1] | 6-chloroquinazoline-2,4-diamine |
| 19 | 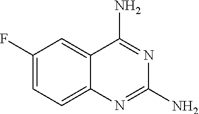 [SMILES: Nc1nc(N)c2cc(F)ccc2n1] | 6-fluoroquinazoline-2,4-diamine |
| 20 | 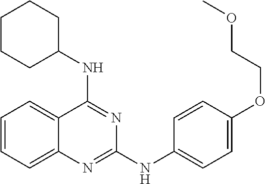 [SMILES: COCCOc1ccc(Nc2nc(NC3CCCCC3)c3ccccc3n2)cc1] | N4-cyclohexyl-N2-(4-(2- methoxyethoxy)phenyl)quinazoline-2,4- diamine |
| 21 | 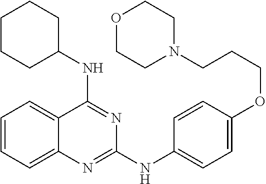 [SMILES: c1ccc2c(NC3CCCCC3)nc(Nc3ccc(OCCCN4CCOCC4)cc3)nc2c1] | N4-cyclohexyl-N2-(4-(3- morpholinopropoxy)phenyl)quinazoline-2,4- diamine |
| 22 | 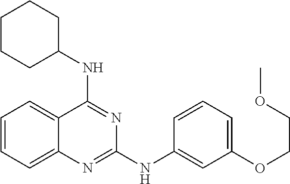 [SMILES: COCCOc1cccc(Nc2nc(NC3CCCCC3)c3ccccc3n2)c1] | N4-cyclohexyl-N2-(3-(2- methoxyethoxy)phenyl)quinazoline-2,4- diamine |
| 23 | 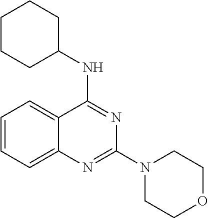 [SMILES: c1ccc2c(NC3CCCCC3)nc(N3CCOCC3)nc2c1] | N-cyclohexyl-2-morpholinoquinazolin-4- amine |
| 24 | 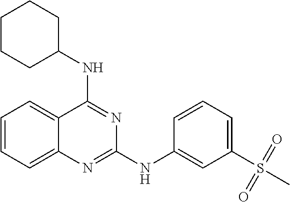 [SMILES: CS(=O)(=O)c1cccc(Nc2nc(NC3CCCCC3)c3ccccc3n2)c1] | N4-cyclohexyl-N2-(3- (methylsulfonyl)phenyl)quinazoline-2,4- diamine |
| 25 | 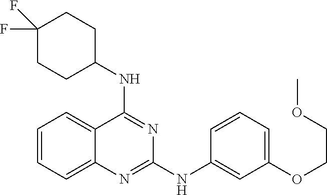 [SMILES: COCCOc1cccc(Nc2nc(NC3CCC(F)(F)CC3)c3ccccc3n2)c1] | N4-(4,4-difluorocyclohexyl)-N2-(3-(2- methoxyethoxy)phenyl)quinazoline-2,4- diamine |
| 26 | 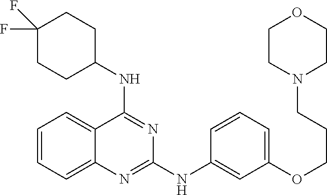 [SMILES: FC1(F)CCC(Nc2nc(Nc3cccc(OCCCN4CCOCC4)c3)nc3ccccc23)CC1] | N4-(4,4-difluorocyclohexyl)-N2-(3-(3- morpholinopropoxy)phenyl)quinazoline-2,4- diamine |
| 27 | 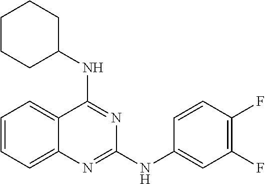 [SMILES: Fc1ccc(Nc2nc(NC3CCCCC3)c3ccccc3n2)cc1F] | N4-cyclohexyl-N2-(3,4- difluorophenyl)quinazoline-2,4-diamine |
| 28 | 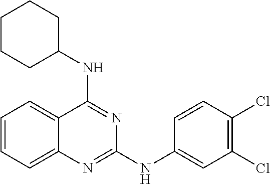 [SMILES: Clc1ccc(Nc2nc(NC3CCCCC3)c3ccccc3n2)cc1Cl] | N4-cyclohexyl-N2-(3,4- dichlorophenyl)quinazoline-2,4-diamine |
| 29 | 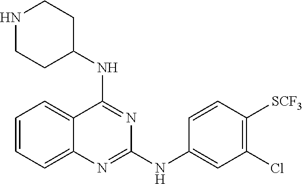 | N2-(3-chloro-4- ((trifluoromethyl)thio)phenyl)-N4- (piperidin-4-yl)quinazoline-2,4-diamine |
| 30 | 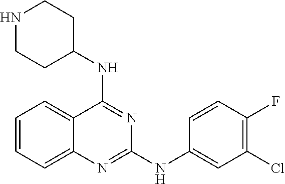 [SMILES: Fc1ccc(Nc2nc(NC3CCNCC3)c3ccccc3n2)cc1Cl] | N2-(3-chloro-4-fluorophenyl)-N4- (piperidin-4-yl)quinazoline-2,4-diamine |
| 31 | 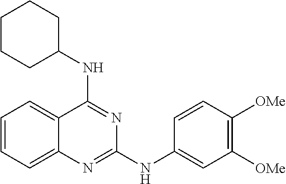 [SMILES: COc1ccc(Nc2nc(NC3CCCCC3)c3ccccc3n2)cc1OC] | N4-cyclohexyl-N2-(3,4- dimethoxyphenyl)quinazoline-2,4-diamine |
| 32 | 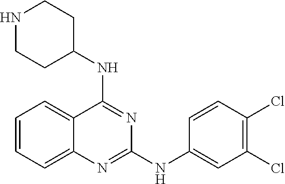 [SMILES: Clc1ccc(Nc2nc(NC3CCNCC3)c3ccccc3n2)cc1Cl] | N2-(3,4-dichlorophenyl)-N4-(piperidin-4- yl)quinazoline-2,4-diamine |
| 33 | 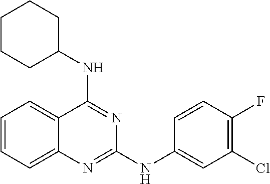 [SMILES: Fc1ccc(Nc2nc(NC3CCCCC3)c3ccccc3n2)cc1Cl] | N2-(3-chloro-4-fluorophenyl)-N4- cyclohexylquinazoline-2,4-diamine |
| 34 | 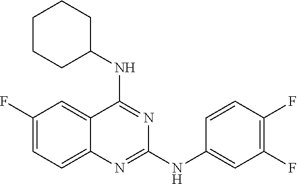 [SMILES: Fc1ccc2nc(Nc3ccc(F)c(F)c3)nc(NC3CCCCC3)c2c1] | N4-cyclohexyl-N2-(3,4-difluorophenyl)-6- fluoroquinazoline-2,4-diamine |
| 35 | 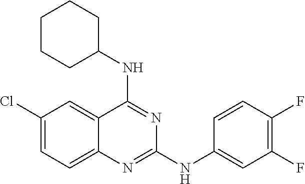 [SMILES: Fc1ccc(Nc2nc(NC3CCCCC3)c3cc(Cl)ccc3n2)cc1F] | 6-chloro-N4-cyclohexyl-N2-(3,4- difluorophenyl)quinazoline-2,4-diamine |
| 36 | 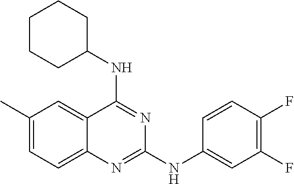 [SMILES: Cc1ccc2nc(Nc3ccc(F)c(F)c3)nc(NC3CCCCC3)c2c1] | N4-cyclohexyl-N2-(3,4-difluorophenyl)-6- methylquinazoline-2,4-diamine |
| 37 | 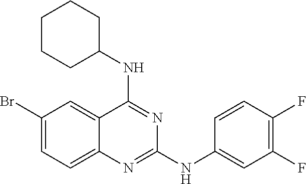 [SMILES: Fc1ccc(Nc2nc(NC3CCCCC3)c3cc(Br)ccc3n2)cc1F] | 6-bromo-N4-cyclohexyl-N2-(3,4- difluorophenyl)quinazoline-2,4-diamine |
| 38 | 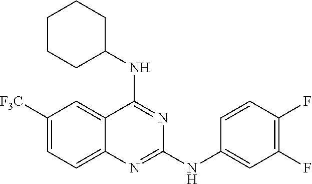 [SMILES: Fc1ccc(Nc2nc(NC3CCCCC3)c3cc(C(F)(F)F)ccc3n2)cc1F] | N4-cyclohexyl-N2-(3,4-difluorophenyl)-6- (trifluoromethyl)quinazoline-2,4-diamine |
| 39 | 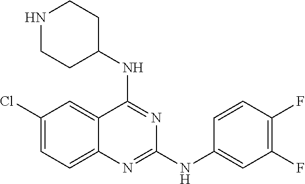 [SMILES: Fc1ccc(Nc2nc(NC3CCNCC3)c3cc(Cl)ccc3n2)cc1F] | 6-chloro-N2-(3,4-difluorophenyl)-N4- (piperidin-4-yl)quinazoline-2,4-diamine |
| 40 | 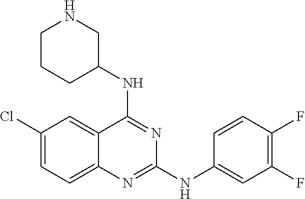 [SMILES: Fc1ccc(Nc2nc(NC3CCCNC3)c3cc(Cl)ccc3n2)cc1F] | 6-chloro-N2-(3,4-difluorophenyl)-N4- (piperidin-3-yl)quinazoline-2,4-diamine |
| 41 | 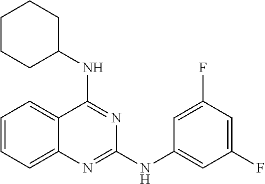 [SMILES: Fc1cc(F)cc(Nc2nc(NC3CCCCC3)c3ccccc3n2)c1] | N4-cyclohexyl-N2-(3,5- difluorophenyl)quinazoline-2,4-diamine |
| 42 | 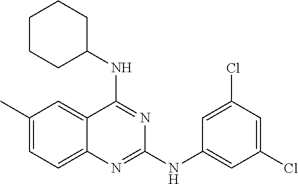 [SMILES: Cc1ccc2nc(Nc3cc(Cl)cc(Cl)c3)nc(NC3CCCCC3)c2c1] | N4-cyclohexyl-N2-(3,5-dichlorophenyl)-6- methylquinazoline-2,4-diamine |
| 43 | 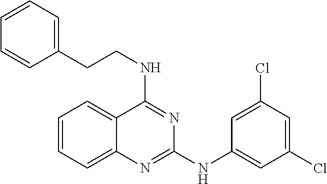 [SMILES: Clc1cc(Cl)cc(Nc2nc(NCCc3ccccc3)c3ccccc3n2)c1] | N2-(3,5-dichlorophenyl)-N4- phenethylquinazoline-2,4-diamine |
| 44 | 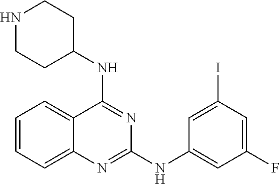 [SMILES: Fc1cc(I)cc(Nc2nc(NC3CCNCC3)c3ccccc3n2)c1] | N2-(3-fluoro-5-iodophenyl)-N4-(piperidin- 4-yl)quinazoline-2,4-diamine |
| 45 | 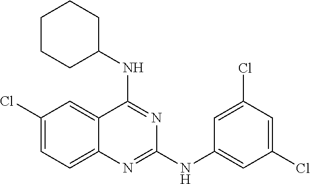 [SMILES: Clc1cc(Cl)cc(Nc2nc(NC3CCCCC3)c3cc(Cl)ccc3n2)c1] | 6-chloro-N4-cyclohexyl-N2-(3,5- dichlorophenyl)quinazoline-2,4-diamine |
| 46 | 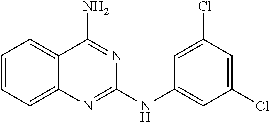 [SMILES: Nc1nc(Nc2cc(Cl)cc(Cl)c2)nc2ccccc12] | N2-(3,5-dichlorophenyl)quinazoline-2,4- diamine |
| 47 | 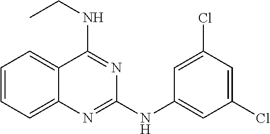 [SMILES: CCNc1nc(Nc2cc(Cl)cc(Cl)c2)nc2ccccc12] | N2-(3,5-dichlorophenyl)-N4- ethylquinazoline-2,4-diamine |
| 48 | 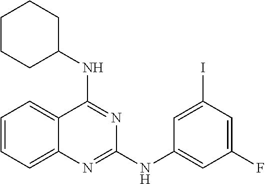 [SMILES: Fc1cc(I)cc(Nc2nc(NC3CCCCC3)c3ccccc3n2)c1] | N4-cyclohexyl-N2-(3-fluoro-5- iodophenyl)quinazoline-2,4-diamine |
| 49 | 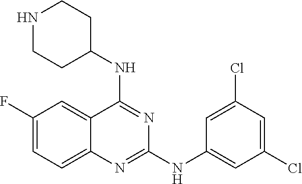 [SMILES: Fc1ccc2nc(Nc3cc(Cl)cc(Cl)c3)nc(NC3CCNCC3)c2c1] | N2-(3,5-dichlorophenyl)-6-fluoro-N4- (piperidin-4-yl)quinazoline-2,4-diamine |
| 50 | 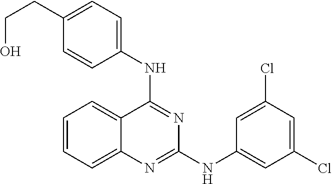 [SMILES: OCCc1ccc(Nc2nc(Nc3cc(Cl)cc(Cl)c3)nc3ccccc23)cc1] | 2-(4-((2-((3,5- dichlorophenyl)amino)quinazolin-4- yl)amino)phenyl)ethan-1-ol |
| 51 | 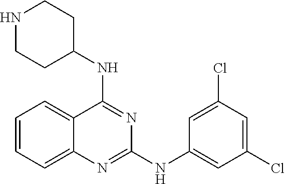 [SMILES: Clc1cc(Cl)cc(Nc2nc(NC3CCNCC3)c3ccccc3n2)c1] | N2-(3,5-dichlorophenyl)-N4-(piperidin-4- yl)quinazoline-2,4-diamine |
| 52 | 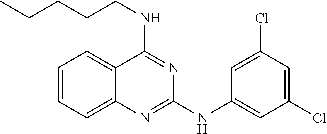 [SMILES: CCCCCNc1nc(Nc2cc(Cl)cc(Cl)c2)nc2ccccc12] | N2-(3,5-dichlorophenyl)-N4- pentylquinazoline-2,4-diamine |
| 53 | 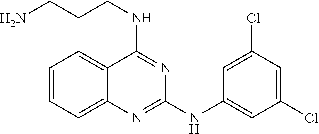 [SMILES: NCCCNc1nc(Nc2cc(Cl)cc(Cl)c2)nc2ccccc12] | N4-(3-aminopropyl)-N2-(3,5- dichlorophenyl)quinazoline-2,4-diamine |
| 54 | 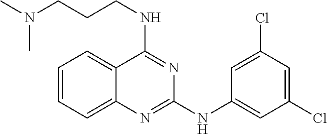 [SMILES: CN(C)CCCNc1nc(Nc2cc(Cl)cc(Cl)c2)nc2ccccc12] | N2-(3,5-dichlorophenyl)-N4-(3- (dimethylamino)propyl)quinazoline-2,4- diamine |
| 55 | 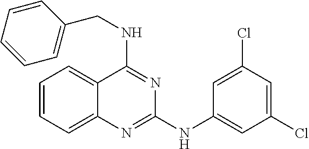 [SMILES: Clc1cc(Cl)cc(Nc2nc(NCc3ccccc3)c3ccccc3n2)c1] | N4-benzyl-N2-(3,5- dichlorophenyl)quinazoline-2,4-diamine |
| 56 | 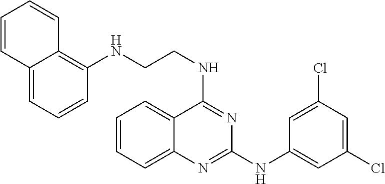 [SMILES: Clc1cc(Cl)cc(Nc2nc(NCCNc3cccc4ccccc34)c3ccccc3n2)c1] | N2-(3,5-dichlorophenyl)-N4-(2-(naphthalen- 1-ylamino)ethyl)quinazoline-2,4-diamine |
| 57 | 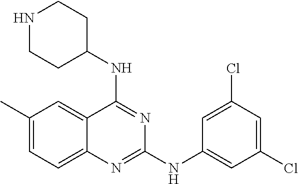 [SMILES: Cc1ccc2nc(Nc3cc(Cl)cc(Cl)c3)nc(NC3CCNCC3)c2c1] | N2-(3,5-dichlorophenyl)-6-methyl-N4- (piperidin-4-yl)quinazoline-2,4-diamine |
| 58 | 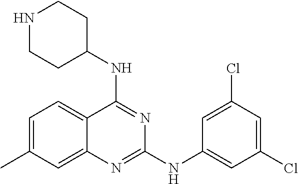 [SMILES: Cc1ccc2c(NC3CCNCC3)nc(Nc3cc(Cl)cc(Cl)c3)nc2c1] | N2-(3,5-dichlorophenyl)-7-methyl-N4- (piperidin-4-yl)quinazoline-2,4-diamine |
| 59 | 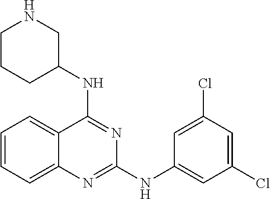 [SMILES: Clc1cc(Cl)cc(Nc2nc(NC3CCCNC3)c3ccccc3n2)c1] | N2-(3,5-dichlorophenyl)-N4-(piperidin-3- yl)quinazoline-2,4-diamine |
| 60 | 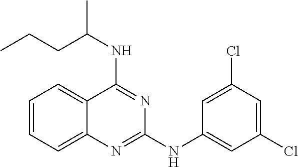 [SMILES: CCCC(C)Nc1nc(Nc2cc(Cl)cc(Cl)c2)nc2ccccc12] | N2-(3,5-dichlorophenyl)-N4-(pentan-2- yl)quinazoline-2,4-diamine |
| 61 | 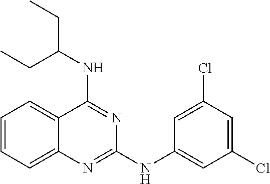 [SMILES: CCC(CC)Nc1nc(Nc2cc(Cl)cc(Cl)c2)nc2ccccc12] | N2-(3,5-dichlorophenyl)-N4-(pentan-3- yl)quinazoline-2,4-diamine |
| 62 | 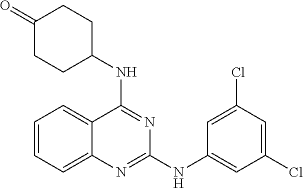 [SMILES: O=C1CCC(Nc2nc(Nc3cc(Cl)cc(Cl)c3)nc3ccccc23)CC1] | 4-((2-((3,5- dichlorophenyl)amino)quinazolin-4- yl)amino)cyclohexan-1-one |
| 63 | 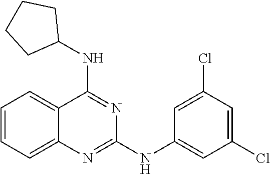 [SMILES: Clc1cc(Cl)cc(Nc2nc(NC3CCCC3)c3ccccc3n2)c1] | N4-cyclopentyl-N2-(3,5- dichlorophenyl)quinazoline-2,4-diamine |
| 64 | 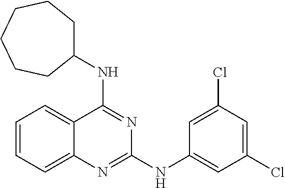 [SMILES: Clc1cc(Cl)cc(Nc2nc(NC3CCCCCC3)c3ccccc3n2)c1] | N4-cycloheptyl-N2-(3,5- dichlorophenyl)quinazoline-2,4-diamine |
| 65 | 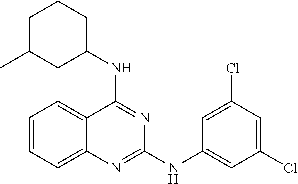 [SMILES: CC1CCCC(Nc2nc(Nc3cc(Cl)cc(Cl)c3)nc3ccccc23)C1] | N2-(3,5-dichlorophenyl)-N4-(3- methylcyclohexyl)quinazoline-2,4-diamine |
| 66 | 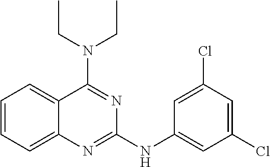 [SMILES: CCN(CC)c1nc(Nc2cc(Cl)cc(Cl)c2)nc2ccccc12] | N2-(3,5-dichlorophenyl)-N4,N4- diethylquinazoline-2,4-diamine |
| 67 | 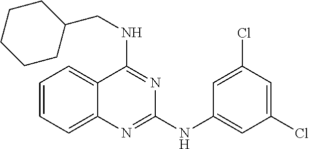 [SMILES: Clc1cc(Cl)cc(Nc2nc(NCC3CCCCC3)c3ccccc3n2)c1] | N4-(cyclohexylmethyl)-N2-(3,5- dichlorophenyl)quinazoline-2,4-diamine |
| 68 | 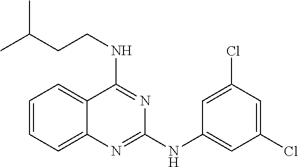 [SMILES: CC(C)CCNc1nc(Nc2cc(Cl)cc(Cl)c2)nc2ccccc12] | N2-(3,5-dichlorophenyl)-N4- isopentylquinazoline-2,4-diamine |
| 69 | 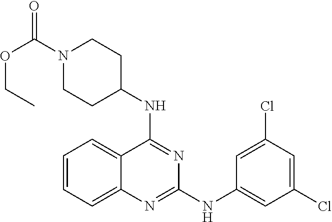 [SMILES: CCOC(=O)N1CCC(Nc2nc(Nc3cc(Cl)cc(Cl)c3)nc3ccccc23)CC1] | ethyl 4-((2-((3,5- dichlorophenyl)amino)quinazolin-4- yl)amino)piperidine-1-carboxylate |
| 70 | 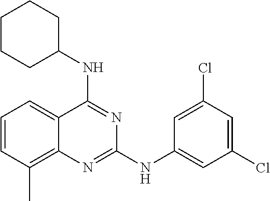 [SMILES: Cc1cccc2c(NC3CCCCC3)nc(Nc3cc(Cl)cc(Cl)c3)nc12] | N4-cyclohexyl-N2-(3,5-dichlorophenyl)-8- methylquinazoline-2,4-diamine |
| 71 | 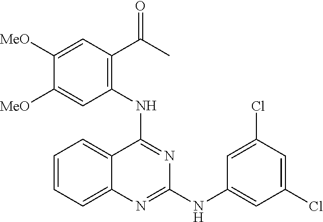 [SMILES: COc1cc(Nc2nc(Nc3cc(Cl)cc(Cl)c3)nc3ccccc23)c(C(C)=O)cc1OC] | 1-(2-((2-((3,5- dichlorophenyl)amino)quinazolin-4- yl)amino)-4,5-dimethoxyphenyl)ethan-1-one |
| 72 | 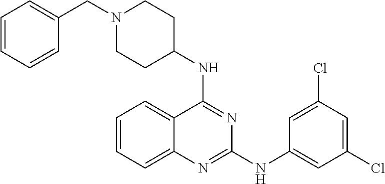 [SMILES: Clc1cc(Cl)cc(Nc2nc(NC3CCN(Cc4ccccc4)CC3)c3ccccc3n2)c1] | N4-(1-benzylpiperidin-4-yl)-N2-(3,5- dichlorophenyl)quinazoline-2,4-diamine |
| 73 | 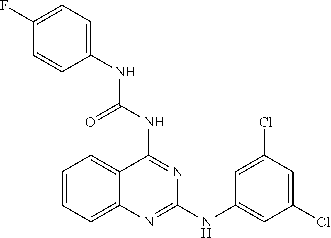 [SMILES: O=C(Nc1ccc(F)cc1)Nc1nc(Nc2cc(Cl)cc(Cl)c2)nc2ccccc12] | 1-(2-((3,5-dichlorophenyl)amino)quinazolin- 4-yl)-3-(4-fluorophenyl)urea |
| 74 | 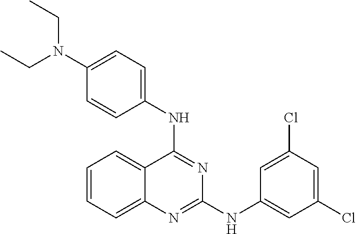 [SMILES: CCN(CC)c1ccc(Nc2nc(Nc3cc(Cl)cc(Cl)c3)nc3ccccc23)cc1] | N2-(3,5-dichlorophenyl)-N4-(4- (diethylamino)phenyl)quinazoline-2,4- diamine |
| 75 | 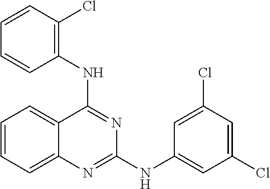 [SMILES: Clc1cc(Cl)cc(Nc2nc(Nc3ccccc3Cl)c3ccccc3n2)c1] | N4-(2-chlorophenyl)-N2-(3,5- dichlorophenyl)quinazoline-2,4-diamine |
| 76 | 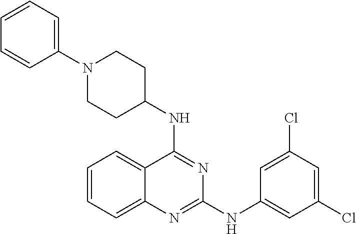 [SMILES: Clc1cc(Cl)cc(Nc2nc(NC3CCN(c4ccccc4)CC3)c3ccccc3n2)c1] | N2-(3,5-dichlorophenyl)-N4-(1- phenylpiperidin-4-yl)quinazoline-2,4- diamine |
| 77 | 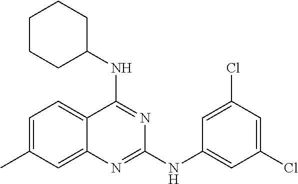 [SMILES: Cc1ccc2c(NC3CCCCC3)nc(Nc3cc(Cl)cc(Cl)c3)nc2c1] | N4-cyclohexyl-N2-(3,5-dichlorophenyl)-7- methylquinazoline-2,4-diamine |
| 78 | 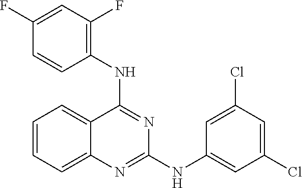 [SMILES: Fc1ccc(Nc2nc(Nc3cc(Cl)cc(Cl)c3)nc3ccccc23)c(F)c1] | N2-(3,5-dichlorophenyl)-N4-(2,4- difluorophenyl)quinazoline-2,4-diamine |
| 79 | 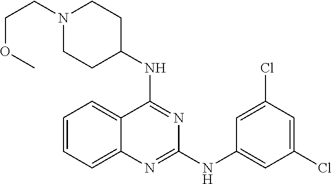 [SMILES: COCCN1CCC(Nc2nc(Nc3cc(Cl)cc(Cl)c3)nc3ccccc23)CC1] | N2-(3,5-dichlorophenyl)-N4-(1-(2- methoxyethyl)piperidin-4-yl)quinazoline- 2,4-diamine |
| 80 | 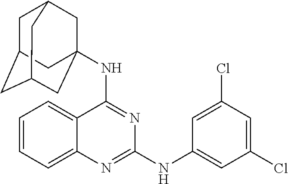 [SMILES: Clc1cc(Cl)cc(Nc2nc(NC34CC5CC(CC(C5)C3)C4)c3ccccc3n2)c1] | N4-((3s,5s,7s)-adamantan-1-yl)-N2-(3,5- dichlorophenyl)quinazoline-2,4-diamine |
| 81 | 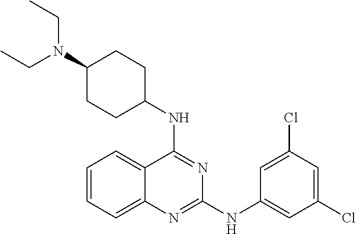 | N2-(3,5-dichlorophenyl)-N4-(4- (diethylamino)cyclohexyl)quinazoline-2,4- diamine |
| 82 | 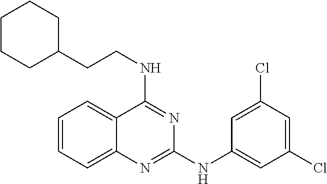 [SMILES: Clc1cc(Cl)cc(Nc2nc(NCCC3CCCCC3)c3ccccc3n2)c1] | N4-(2-cyclohexylethyl)-N2-(3,5- dichlorophenyl)quinazoline-2,4-diamine |

In some embodiments, the negative allosteric modulator of β₂AR comprises a compound of formula (1) and a compound of formula (2). In other embodiments, the negative allosteric modulator of β₂AR comprises a compound of formula (1) and a compound of formula (3). In yet another embodiment, the negative allosteric modulator of β₂AR comprises a compound of formula (2) and a compound of formula (3). In yet another embodiment, the negative allosteric modulator of β₂AR comprises a compound of formula (1), a compound of formula (2), and a compound of formula (3).

The compounds of the present disclosure may be prepared using methods known to those skilled in the art of organic synthesis in view of the disclosures of International Patent Application Publication Nos. WO2006105056A2, WO199207844A1, WO2011140527A2, and WO2015073836A1, and/or Chou et al. (ChemMedChem, 2013, 8(2):297-312), which are hereby incorporated by reference herein in their entireties.

The compounds of the disclosure may possess one or more stereocenters, and each stereocenter may exist independently in either the (R)- or (S)-configuration. In certain embodiments, compounds described herein are present in optically active or racemic forms. The compounds described herein encompass racemic, optically active, regioisomeric and stereoisomeric forms, or combinations thereof that possess the therapeutically useful properties described herein. Preparation of optically active forms is achieved in any suitable manner, including, by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or any mixtures thereof, or in the case where two or more chiral centers are present, all diastereomers or any mixtures thereof.

In certain embodiments, the compounds of the disclosure exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature.

Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²F, and ³⁵S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In all of the embodiments provided herein, examples of suitable optional substituents are not intended to limit the scope of the claimed disclosure. The compounds of the disclosure may contain any of the substituents, or combinations of substituents, provided herein.

### Salts

The compounds described herein may form salts with acids or bases, and such salts are included in the present disclosure. The term “salts” embraces addition salts of free acids or bases that are useful within the methods of the disclosure. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present disclosure, such as for example utility in process of synthesis, purification or formulation of compounds useful within the methods of the disclosure.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, O-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the disclosure.

Suitable pharmaceutically acceptable base addition salts of compounds of the disclosure include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

In certain embodiments, the compounds described herein can be administered to the subject as part of a pharmaceutical composition that further includes at least one pharmaceutical excipient. The pharmaceutical composition may be formulated for administration by any method known in the art. In certain embodiments, the pharmaceutical composition is formulated for inhalational administration. Further details of dose and administration are discussed elsewhere herein.

### Administration/Dosage/Formulations

The regimen of administration may affect what constitutes an effective amount. The therapeutic formulations may be administered to the subject either prior to or after the onset of a disease or disorder contemplated in the disclosure. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, preferably a mammal, more preferably a human, may be carried out using known procedures, at dosages and for periods of time effective to treat a disease or disorder contemplated in the disclosure. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated in the disclosure. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the disclosure is from about 1 and 5,000 mg/kg of body weight/per day. The pharmaceutical compositions useful for practicing the disclosure may be administered to deliver a dose of from ng/kg/day and 100 mg/kg/day. One of ordinary skill in the art would be able to study the relevant factors and make the determination regarding the effective amount of the therapeutic compound without undue experimentation.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder contemplated in the disclosure.

In certain embodiments, the compositions of the disclosure are formulated using one or more pharmaceutically acceptable excipients or carriers. In other embodiments, the pharmaceutical compositions of the disclosure comprise a therapeutically effective amount of a compound of the disclosure and a pharmaceutically acceptable carrier. In yet other embodiments, the compound of the disclosure is the only therapeutically effective agent administered to the subject. In yet other embodiments, the compound of the disclosure is the only therapeutically effective agent in the composition.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the disclosure are administered to the patient in dosages that range from one to five times per day or more. In other embodiments, the compositions of the disclosure are administered to the patient in range of dosages that include, but are not limited to, once every day, every two days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Compounds of the disclosure for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments there between.

In some embodiments, the dose of a compound of the disclosure is from about 1 mg and about 2,500 mg. In some embodiments, a dose of a compound of the disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder contemplated in the disclosure.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., anti-fibrotic agents.

Routes of administration of any of the compositions of the disclosure include oral, nasal, rectal, intravaginal, parenteral, buccal, sublingual or topical. The compounds for use in the disclosure may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the disclosure may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulfate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e., powder or granular) form for reconstitution with a suitable vehicle (e.g., sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Inhalational Administration and the Like

Routes of administration of any of the compositions of the disclosure include nasal, inhalational, intratracheal, intrapulmonary, and intrabronchial.

Suitable compositions and dosage forms include, for example, dispersions, suspensions, solutions, syrups, granules, beads, powders, pellets, liquid sprays for nasal or oral administration, dry powder or aerosolized formulations for inhalation, and the like. It should be understood that the formulations and compositions that would be useful in the present disclosure are not limited to the particular formulations and compositions that are described herein.

Powdered and granular formulations of a pharmaceutical preparation of the disclosure may be prepared using known methods. Such formulations may be administered directly to a subject, used, for example, to form a material that is suitable to administration to a subject. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the disclosure may be prepared, packaged, or sold in a formulation suitable for pulmonary administration via the buccal cavity. Such a formulation may comprise dry particles that comprise the active ingredient and have a diameter in the range from about 0.5 to about 7 micrometers, and preferably from about 1 to about 6 micrometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 micrometers and at least 95% of the particles by number have a diameter less than 7 micrometers. More preferably, at least 95% of the particles by weight have a diameter greater than 1 nanometer and at least 90% of the particles by number have a diameter less than 6 micrometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the disclosure formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 micrometers.

The pharmaceutical composition of the disclosure may be delivered using an inhalator such as those recited in U.S. Pat. No. 8,333,192 B2, which is incorporated herein by reference in its entirety.

The formulations described herein as being useful for pulmonary delivery are also useful for intranasal delivery of a pharmaceutical composition of the disclosure.

Another formulation suitable for intranasal administration is a coarse powder comprising the active ingredient and having an average particle from about 0.2 to 500 micrometers. Such a formulation is administered in the manner in which snuff is taken, i.e. by rapid inhalation through the nasal passage from a container of the powder held close to the nares. Formulations suitable for nasal administration may, for example, comprise from about as little as 0.1% (w/w) and as much as 100% (w/w) of the active ingredient, and may further comprise one or more of the additional ingredients described herein.

Additional Administration Forms

Additional dosage forms of this disclosure include dosage forms as described in U.S. Pat. Nos. 6,340,475; 6,488,962; 6,451,808; 5,972,389; 5,582,837; and 5,007,790. Additional dosage forms of this disclosure also include dosage forms as described in U.S. Patent Applications Nos. 20030147952; 20030104062; 20030104053; 20030044466; 20030039688; and 20020051820. Additional dosage forms of this disclosure also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

Controlled Release Formulations and Drug Delivery Systems

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material that provides sustained release properties to the compounds. As such, the compounds for use the method of the disclosure may be administered in the form of microparticles, for example, by injection or in the form of wafers or discs by implantation.

In certain embodiments, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Dosing

The therapeutically effective amount or dose of a compound of the present disclosure depends on the age, sex and weight of the patient, the current medical condition of the patient and the progression of a disease or disorder contemplated in the disclosure. The skilled artisan is able to determine appropriate dosages depending on these and other factors.

A suitable dose of a compound of the present disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the disclosure is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In certain embodiments, patients require intermittent treatment on a long-term basis upon any recurrence of symptoms and/or infection.

The compounds for use in the method of the disclosure may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, and reducing/oxidizing agents, with art-recognized alternatives and using no more than routine experimentation, are within the scope of the present application. It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### EXPERIMENTAL EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

### Methods

Cell culture GLOSENSOR™ cells (Promega) were maintained in Dulbecco's modified Eagle's medium (DMEM) containing 10% fetal bovine serum (FBS) and 200 μg/ml Hygromycin B. PATHHUNTER® ADRB2 cells (DiscoveRx) were maintained with the AssayComplete™ Cell Culture kit (DiscoveRx catalog #92-3107G). HEK 293 cells were maintained in DMEM containing 10% FBS and 12.5 mM HEPES, pH 7.2. HEK 293 cells stably overexpressing β₂AR included 250 μg/ml G418. Cells were incubated at 37° C. in a humidified incubator with 5% CO₂.
High throughput assay for cAMP production. 6,250 GLOSENSOR™ cells in 25 μL of CO₂-independent medium (Invitrogen catalog #18045088) supplemented with 10% FBS were plated per well in sterile, white, tissue-culture treated 384-well plates and allowed to attach overnight at 37° C. in 5% CO₂ atmosphere. Plates were removed from the incubator, 25 μL of CO₂-independent medium, 10% FBS and 44% v/v GLOSENSOR™ cAMP reagent (Promega) was added to each well and plates were allowed to equilibrate to room temperature for two hours. 0.25 μL of orthogonally compressed libraries from the Lankenau Chemical Genomics Center (LCGC) were transferred into plates using a high-density replication tool and allowed to incubate with cells for one hour at 37° C. Cells were read every three minutes for 18 minutes on a PolarStar Optima plate reader (BMG) to establish peak signal. ISO was added to each well to a final concentration of 1 μM, and luminescence was determined in kinetic mode at room temperature (23-25° C.). Response was analyzed relative to forskolin (7 μM) and alprenolol (1 μM) as positive and negative controls, respectively.
High throughput assay for β-arrestin recruitment to β₂AR. 5,000 PATHHUNTER® ADRB2 cells in 20 μL of AssayComplete™ cell plating reagent (DiscoveRx catalog #93-0563R2A) were plated per well in sterile, white, tissue-culture treated 384-well plates and allowed to attach overnight at 37° C. in 5% CO₂ atmosphere. 0.25 μL of orthogonally compressed libraries from the Lankenau Chemical Genomics Center (LCGC) were transferred into plates using a high-density replication tool and allowed to incubate with cells for 45 minutes at 37° C. ISO (5 μL of 5 μM stock in cell plating medium) was added to each well to a final concentration of 1 μM, and cells were incubated for 70 minutes at 37° C. Plates were removed from the incubator, and 12.5 μL of PATHHUNTER® detection solution (DiscoveRx catalog #93-0001) was added to each well. Plates were incubated in the dark at room temperature for one hour. Luminescence was determined at room temperature (23-25° C.) on a PolarStar Optima plate reader (BMG). Response was analyzed relative to vehicle (DMSO) and alprenolol (1 μM) as positive and negative controls, respectively.
Screening data analysis and IC₅₀ determination. Negative control signal was subtracted from all data and responses were normalized relative to full response (positive control minus negative control). Relative assay bias was calculated from the ratio of normalized cAMP and arrestin recruitment responses. Wells from orthogonally-compressed libraries displaying Gₛ-bias (negative allosteric modulators of arrestin recruitment and positive allosteric modulators of cAMP production, excluding inverse agonists) were deconvoluted to identify compounds for follow-up analysis. Candidate compounds were titrated from 10 μM to 10 nM concentration in half-logarithmic steps in GLOSENSOR™ and PATHHUNTER® assays in order to determine IC₅₀ values for cAMP production and β-arrestin recruitment. Compounds displaying confirmed Gₛ-bias were progressed to secondary screening.
cAMP measurement. HEK 293 cells stably overexpressing β₂AR were seeded in poly-L-lysine coated 24-well plates and incubated at 37° C. Cells were pretreated with 0.1% DMSO or 10 μM DFPQ for 30 min prior to stimulation with indicated concentrations of ISO for 10 min. Cells were then lysed in 0.1 M HCl for 20 min at room temperature. cAMP levels were measured using the Caymen Chemical Cyclic AMP EIA kit following the manufacturer's instructions.
Analysis of/3-arresting binding to the β₂AR using bioluminescence resonance energy transfer (BRET). HEK 293 cells were transfected with pcDNA-O-arrestin2-GFP10 and either pcDNA3-β₂AR-RlucII, pcDNA3-β₁AR-RlucII or pcDNA3-CXCR4-RlucII using X-tremegene HP9 complexed in serum-free optiMEM. 24 hr after transfection, cells were replated at 100,000 cells per well in an opaque, poly-L-lysine coated 96-well plate and incubated overnight at 37° C. Cells were then pretreated with 0.1% DMSO or DFPQ for 30 min followed by the addition of Coelenterazine 400a and agonist stimulation. BRET was measured using a Tecan Infinite F500 microplate reader. BRET ratios were calculated as the light emitted by the GFP10 acceptor divided by the total light emitted by the RLucII donor.
Analysis of Gₛ activation by GTPγS binding. 18 μM of purified Gₛ heterotrimer in 2% 3:1 dimyristoyl phosphatidylcholine (DOPC):3-([3-cholamidopropyl]dimethylammonio)-2-hydroxy-1-propanesulfonate (CHAPSO) bicelles with 1.13 mM cholesterol hemisuccinate (CHS), 20 mM HEPES, pH 7.5, and 100 mM NaCl was incubated in the presence of 1.5 μM β₂AR for 2 hr on ice to allow protein incorporation into the lipid bicelles. 2 μl of reconstituted β₂AR-Gₛ was diluted 200-fold in 20 mM HEPES, pH 7.5, 150 mM NaCl, 1 mM MgCl₂ and 38.5 nM [³⁵S]GTPγS with or without 10 μM DFPQ and incubated for 30 min. 20 μl reactions were initiated by the addition of 1 μM ISO and incubated for 15 min at room temperature while negative control samples had no ISO. Bound [35 S]GTPγS was collected by rapid filtration on GF/B filters, washed 4 times with 4 ml of cold GTPγS wash buffer and analyzed by liquid scintillation counting.
In-cell β₂AR phosphorylation. HEK 293 cells stably overexpressing FLAG-β₂AR were seeded into poly-L-lysine coated 6-well plates and incubated at 37° C. Cells were pretreated with 0.1% DMSO or indicated concentrations of DFPQ for 30 min prior to stimulation with 1 μM ISO for 10 min. Cells were then lysed on ice, scraped, and sonicated. Lysates were immunoprecipitated using rabbit polyclonal anti-FLAG and Protein G agarose beads. Immunoprecipitated proteins were separated by SDS-PAGE and analyzed by western blot using a β₂AR C-terminal tail or phospho-specific antibody.
In vitro tubulin phosphorylation assay. GRK2 and GRK5-mediated phosphorylation of tubulin was assayed by incubating purified kinase (50 nM) and tubulin (1.5 μM) in 20 mM Tris-HCl, pH 7.4, 5 mM MgCl₂, 30 mM NaCl, 0.5 mM EDTA, 100 μM [γ³²P]ATP (2000 cpm/pmol) at 30° C. for the indicated times. After incubation, reactions were stopped with SDS sample buffer and the samples were electrophoresed on 10% SDS-polyacrylamide gels and visualized by autoradiography. Band intensity was measured by densitometry using ImageJ.
Receptor internalization. HEK 293 cells stably expressing FLAG-β₂AR were seeded into poly-L-lysine coated 24-well plates and incubated at 37° C. Cells were pretreated with 0.1% DMSO or 10 μM DFPQ for 30 min prior to stimulation with 1 μM ISO for 0-60 min. Cells were then fixed on ice and processed for cell surface ELISA with polyclonal anti-FLAG primary antibody, anti-rabbit HRP secondary antibody, and incubation with (2,2′-Azinobis [3-ethylbenzothiazoline-6-sulfonic acid]-diammonium salt) (ABTS). Absorbance was then measured on a plate reader at 405 nm.
Functional desensitization in HEK 293 cells. HEK 293 cells were seeded in poly-L-lysine coated 24-well plates and incubated at 37° C. Cells were incubated with 0.1% DMSO or 10 μM DFPQ for 30 min prior to stimulation with 1 μM ISO for 30 min. Cells were washed 3 times with PBS and incubated with various concentrations of ISO for 10 min at 37° C. Cells were then lysed in 0.1 M HCl for 20 min at room temperature and cAMP levels were measured using the Caymen Chemical Cyclic AMP EIA kit following the manufacturer's instructions.
Measurement of airway contractility. For ex vivo evaluation, lungs were harvested from mouse strain C₅₇BL/6 (10-16 weeks old). Tracheotomy was performed for cannulation to gain access to lungs. The thoracic cavity was exposed to detach lung tissue from the diaphragm to allow for space for lungs to expand. Warm molten low melting point agarose (2-4% w/v, ˜1 ml total volume) was injected into murine lungs through the cannula using a 1 ml syringe. Lungs were monitored for appropriate inflation. Following this, ˜0.2 ml of air was injected into the expanded lungs and mice were placed at 4° C. for 30-45 min to allow for agarose to solidify. At the end of incubation, the lung tissue was excised and the left lung lobe was processed for generation of lung tissue slices using an OTS-5000 tissue slicer. On day 1, airway tissue was contracted with 1 μM methacholine and then relaxed with 1 μM ISO±1 μM DFPQ overnight. On day 2, tissue was washed, re-contracted with methacholine, and then re-challenged with ISO. Luminal airway was monitored by microscopy over the indicated time course and images were collected at various time points indicated in the results. Images were analyzed post hoc using the ImageJ software.
Human airway smooth muscle cell scratch assay. HASM cells were seeded into 24-well plates, and a line was scratched in the center of the cell monolayer using a sterile 200 μl pipette tip and then washed three times with PBS to remove the cell debris. Migration of HASM cells into the cleared area was determined at 0 and 24 hours in the presence of PDGF-BB (20 ng/ml) stimulation. All images were captured by an EVOS FL Auto Cell Imaging System inverted microscope (Life Technologies, Carlsbad, CA). Cell-free area was quantitated using ImageJ.
Quantification and statistical analysis. All statistical analyses were produced using Prism 8.0 (GraphPad Software). All data are expressed as the mean±standard error of the mean (SEM), unless otherwise stated in the figure legend.

### Example 1: Identifying Small-Molecule Allosteric Modulators of the β₂AR

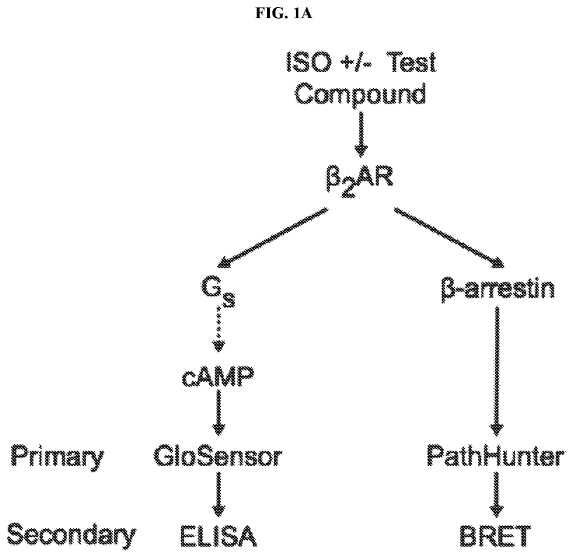

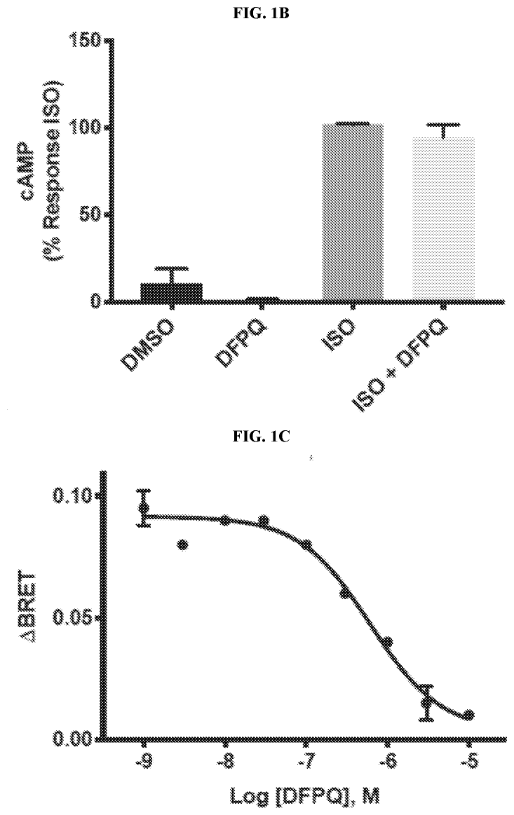

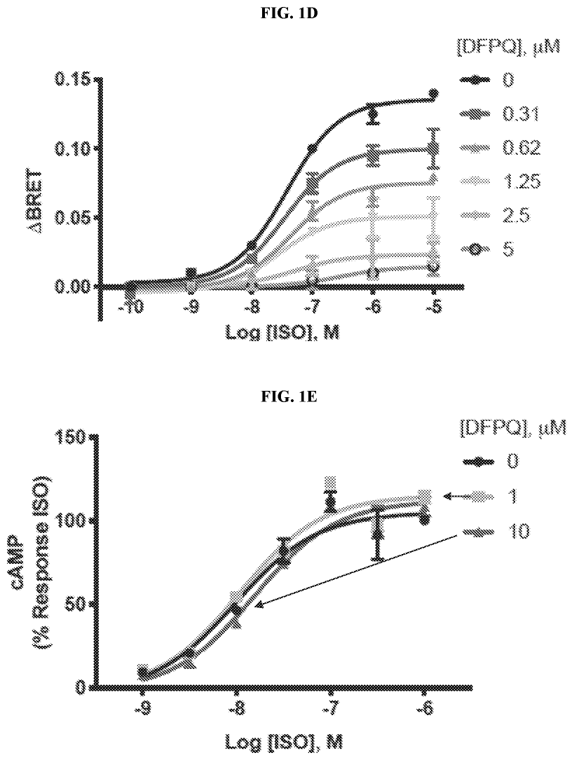

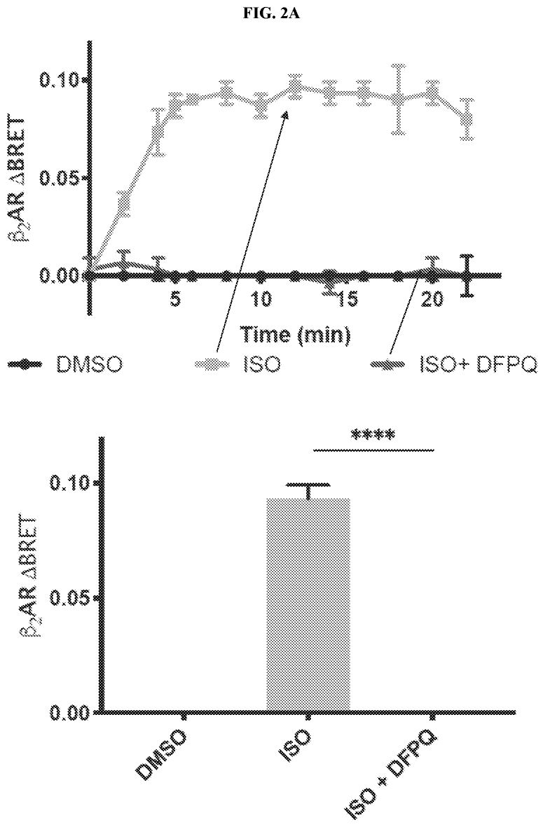

In an effort to discover novel small-molecule allosteric modulators of the β₂AR, compounds were screened in orthogonal primary and secondary assays for cAMP production and β-arrestin2 recruitment to the β₂AR. In primary screens, cells expressing β₂AR were stimulated with the agonist ISO in the presence or absence of 10 NM test compounds and evaluated for cAMP production using the GLOSENSOR™ assay and β₂AR/β-arrestin2 association using the PATHHUNTER® ADRB2 assay. Hits from primary screening were then titrated in GLOSENSOR™ and PATHHUNTER® assays in order to identify compounds capable of inhibiting ISO-promoted β-arrestin2 recruitment in a dose-dependent manner with minimal effect on cAMP production. The most potent compounds displaying NAM activity in the primary screens were further characterized by competitive ELISA to measure cAMP production and bioluminescence resonance energy transfer (BRET) to analyze β-arrestin2 recruitment to the β₂AR. The orthogonal nature of the detection methods used for primary and secondary screening allowed us to confirm compound-induced phenotypes and rule out false positives arising from assay artifacts. This assay schema is summarized in FIG. 1A.

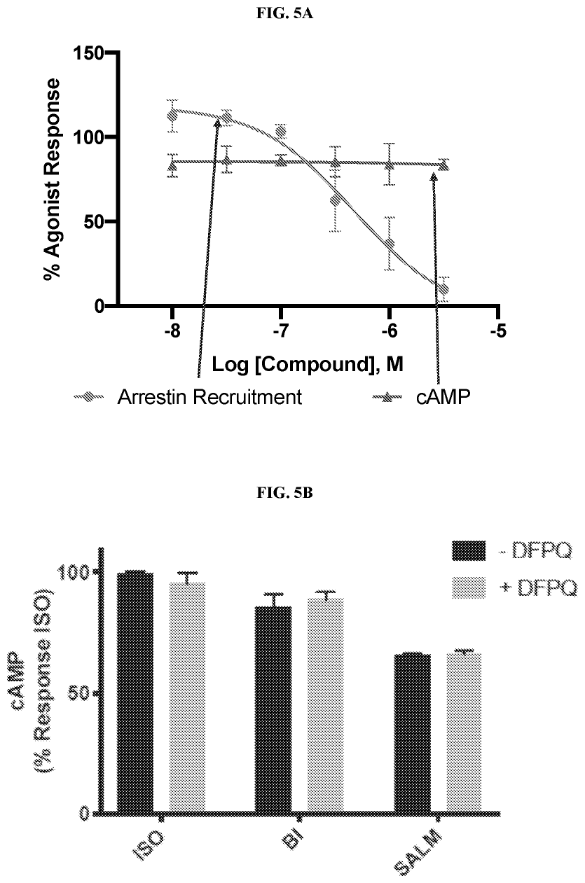

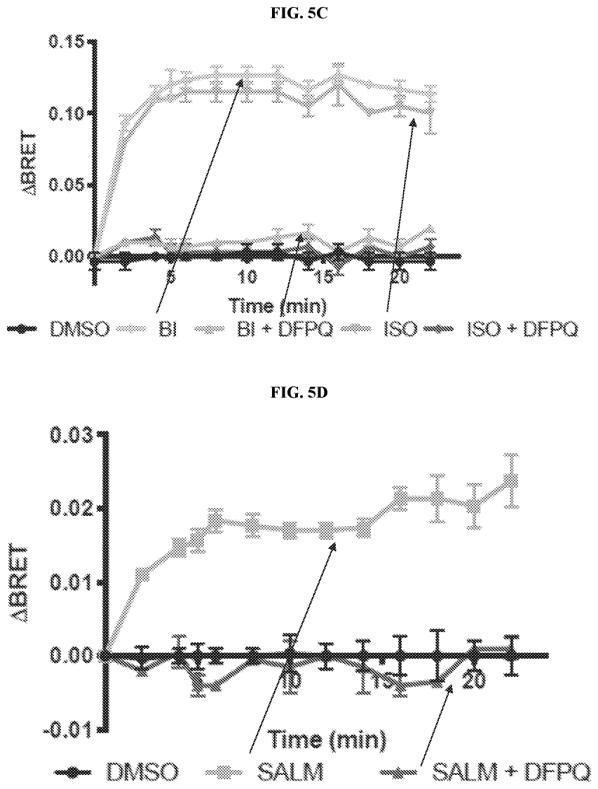

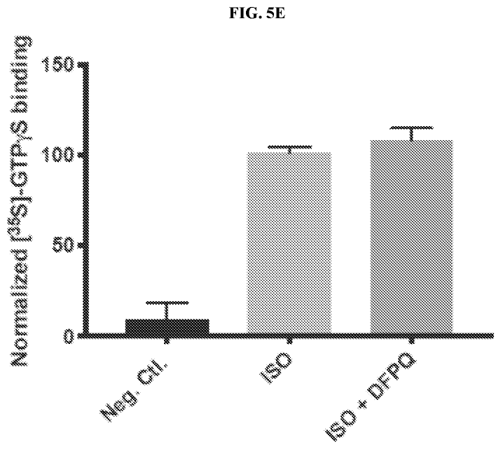

Primary screening of diverse small molecule libraries totaling more than 152,000 compounds identified 578 compounds that scored as either β-arrestin NAMs or positive allosteric modulators (PAMs) of ISO-stimulated cAMP production. Titration of these hits confirmed 57 compounds that displayed biased inhibition of ISO-mediated β-arrestin2 recruitment to the β₂AR relative to ISO-stimulated cAMP production. Many of these compounds were quinazoline derivatives that demonstrated the desired signaling properties when further investigated in secondary assays. The most potent of these compounds, a (3,4-difluorophenyl) quinazoline derivative (DFPQ), selectively inhibited β-arrestin2 binding in the primary (FIG. 5A) and secondary screens with no effect on ISO-stimulated cAMP production (FIG. 1B) and potent inhibition of β-arrestin binding to the β₂AR with an IC₅₀ of ˜0.6 μM (FIG. 1C). This signaling profile was maintained for pharmacologically distinct β-agonists including the full agonist BI-167107 and the partial agonist salmeterol (FIGS. 7B-7D). Thus, the screening identified DFPQ as a strongly biased modulator of β-arrestin recruitment to the β₂AR.

To further confirm an allosteric mechanism of action, a combination of functional assays was used to pharmacologically profile the interaction of DFPQ with the β₂AR. Typically, functional readouts of negative allosteric modulators would be expected to demonstrate a decreased maximal response to an orthosteric agonist in the presence of increasing concentrations of the modulator. These properties were evaluated for ISO dose response curves in cells treated with increasing concentrations of DFPQ. In order to incorporate the biased nature of the effects of DFPQ on β₂AR signaling, these experiments were performed for both β-arrestin recruitment and cAMP production. Using β-arrestin recruitment as a functional readout, ISO dose response curves demonstrate the classical decreased response typical of NAMs at increasing concentrations of DFPQ (FIG. 1D). Global curve fitting and Schild analysis of these data demonstrate non-competitive inhibition, a strong indicator of an allosteric mechanism of action for this response. Notably, increasing concentrations of DFPQ have no effect on ISO dose response curves using cAMP production as a functional readout and incubation with 10 μM DFPQ (FIG. 1E). In addition, DFPQ had no effect on ISO-dependent activation of purified Gₛ by the β₂AR as assessed by radiolabeled GTPγS binding (FIG. 5E). These results suggest that in the presence of ISO and DFPQ, the efficacy for β-arrestin recruitment is diminished while efficacy for Gₛ activation is unaffected, and indicate that DFPQ is a β-arrestin biased negative allosteric modulator.

### Example 2: Receptor Specificity of DFPQ

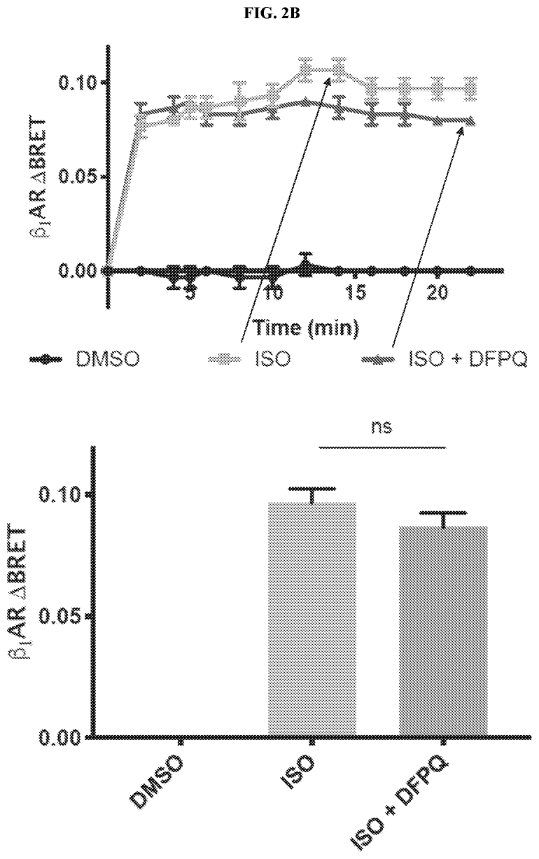

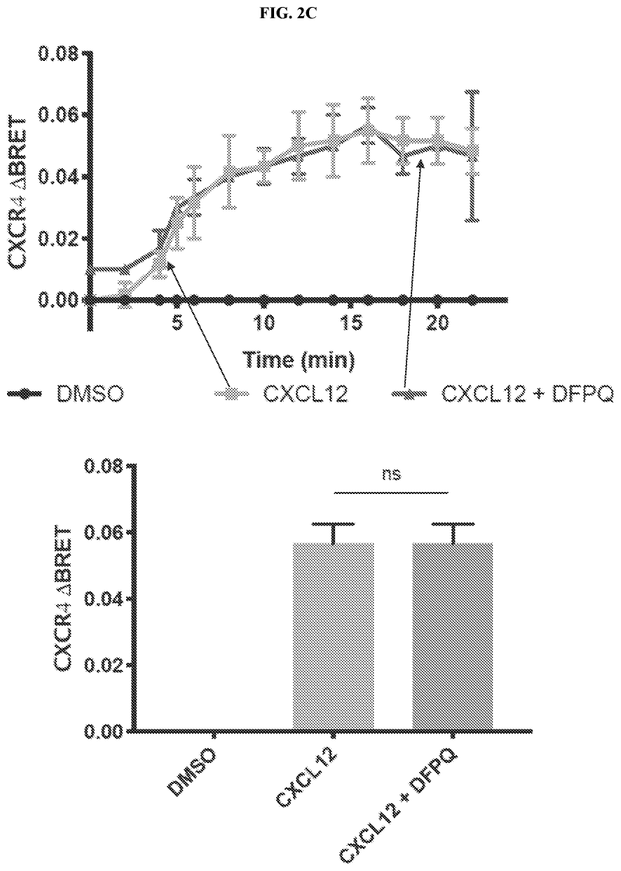

Novel therapeutic compounds often have undesirable side effects due to off-target interactions. A goal of investigating β₂AR allosteric modulators is the potential high degree of receptor specificity that can be achieved when targeting domains outside of the endogenous ligand-binding site. Domains that have not co-evolved with receptor family members for binding endogenous ligands are less conserved, and can therefore be used to chemically discriminate between related receptors. To evaluate receptor specificity, BRET was used to measure the effect of DFPQ on β-arrestin interaction with the β₂AR, β₁AR and CXCR4. Evaluating both the peak BRET signal and extended time course for agonist-promoted β₂AR/β-arrestin2 interaction shows that β-arrestin2 recruitment to the β₂AR is fully inhibited by 10 μM DFPQ over the measured time course (FIG. 2A). In contrast, β-arrestin2 interaction with the β₁AR, the most homologous GPCR to the β₂AR, is not significantly inhibited by 10 μM DFPQ (FIG. 2B). Additionally, there was also no effect of DFPQ on (3-arrestin2 interaction with CXCR4 (FIG. 2C). Collectively, these data demonstrate that DFPQ is highly selective for the β₂AR and is acting through a receptor specific mechanism rather than broadly inhibiting β-arrestin interaction with GPCRs.

### Example 3: Structure Activity Relationship of Quinazolines and Inhibition of 13-Arrestin Recruitment

The chemical structure of DFPQ represents a novel chemical space for β₂AR ligands. To assess structure activity relationships of DFPQ, chemically related quinazoline structures present in the primary compound library were evaluated by dose response analysis using the screening assays shown in FIG. 1A. From these studies, it was determined that R₁ substitutions of the difluorophenyl head group modulated efficacy and affinity (Table 2 and Table 3). The most important substituent for efficacy is the para-fluoro moiety at the 4 position of the phenyl group with 4-fluoro phenyl substitution demonstrating the highest efficacy for inhibition of β-arrestin recruitment. An unmodified phenyl ring is −10-fold less potent while chloro substituents of the phenyl group also show a significant decrease in efficacy particularly at the meta position. Most substitutions of the phenyl ring with other groups also significantly reduced potency. R₃ substitutions of the cyclohexane group were determined to be a potential driver of signaling bias, however, R₃ substitutions containing the difluorophenyl head group were poorly represented in the library, making direct comparisons difficult (Table 3). Nevertheless, all substitutions of the cyclohexane reduced potency and all had reduced β-arrestin bias. R₂ substitutions of the quinazoline ring scaffold were also poorly represented in the library although addition of a chloro to the quinazoline at position 6 improved potency while modifications at position 8 significantly reduced potency and bias (Table 2). Synthesis of compounds containing a difluorophenyl at R₁ with modifications at R₂ identified a number of higher affinity compounds with either a bromo or trifluoromethyl addition to the quinazoline at position 6 (Table 2).

### Example 4: DFPQ Inhibits GRK-Mediated Phosphorylation of the β₂AR

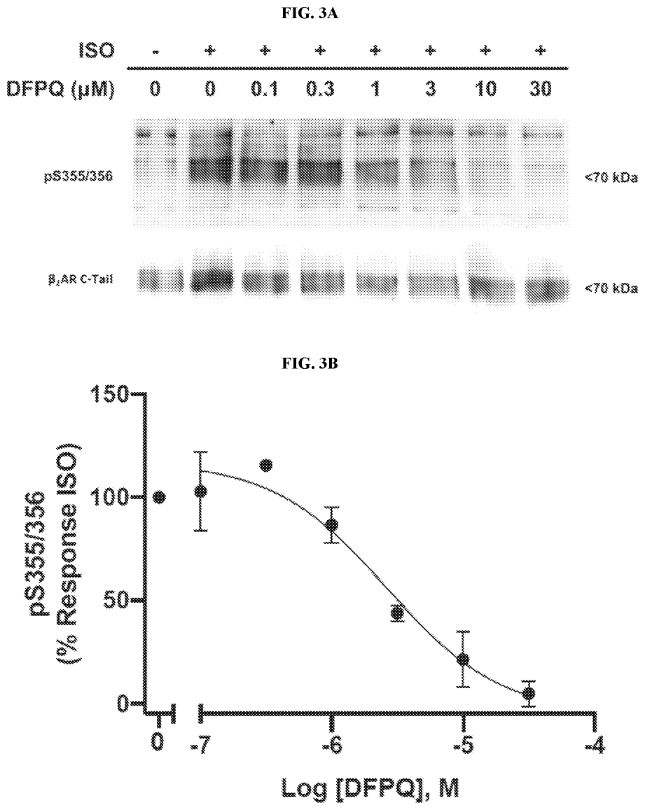

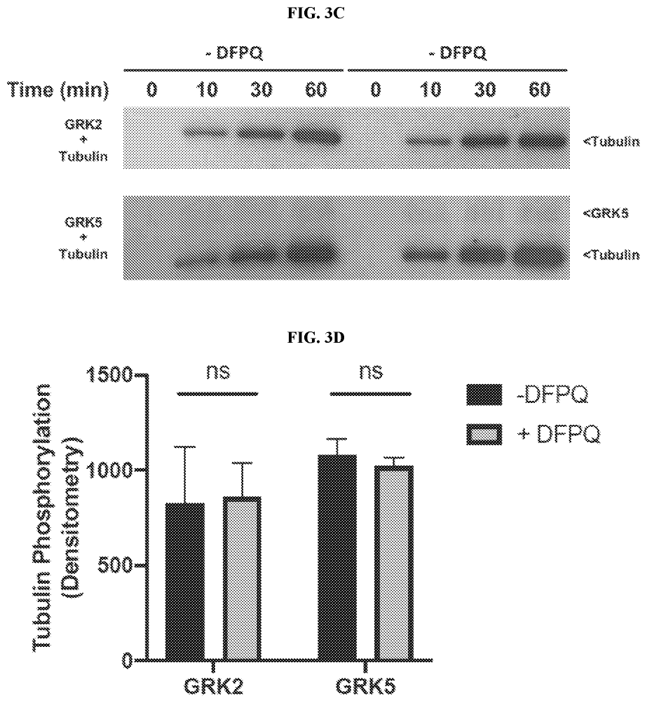

G protein-coupled receptor kinases (GRKs) play a role in orchestrating biased agonism at the β₂AR as receptor phosphorylation is a prerequisite for the recruitment of (3-arrestin. To investigate the mechanistic role of β₂AR phosphorylation by GRKs on the observed signaling phenotype demonstrated by DFPQ, the effect of DFPQ on agonist-promoted phosphorylation of the β₂AR was evaluated. In-cell phosphorylation of the β₂AR was examined in HEK 293 cells expressing FLAG-β₂AR and was monitored using a phosphospecific antibody targeting PSer^{355/356}, a primary site of GRK-mediated phosphorylation in the β₂AR. The cells were stimulated with ISO in the presence of a range of DFPQ concentrations. Treatment with ISO alone induced robust phosphorylation of the β₂AR, while DFPQ effectively inhibited ISO-stimulated phosphorylation with an IC₅₀ of 2.6 μM±1.0 μM (FIGS. 3A3B).

To help ensure that the inhibition of GRK-mediated phosphorylation of the β₂AR was not a function of inhibiting GRK catalytic activity, the effect of DFPQ on GRK2 and GRK5 phosphorylation of a shared substrate, tubulin, was measured in vitro using purified kinases and substrate. Phosphorylation of tubulin by GRK2 or GRK5 was unaffected by DFPQ treatment over a 60-minute time course (FIG. 3C). Three experiments were quantified at the 60-minute timepoint (FIG. 3D), revealing that GRK catalytic activity is unaffected by DFPQ treatment. This is also supported by the lack of an effect of DFPQ on GRK5 autophosphorylation (FIG. 3C).

These results suggest that DFPQ may act by stabilizing a conformation of the β₂AR that is unfavorable for GRK-mediated phosphorylation while also demonstrating that DFPQ has no direct effect on GRK catalytic activity. The disruption of β₂AR phosphorylation by DFPQ likely contributes to the observed β-arrestin bias of this compound.

### Example 5: DFPQ Treatment Antagonizes Agonist-Promoted β₂AR Internalization and Desensitization

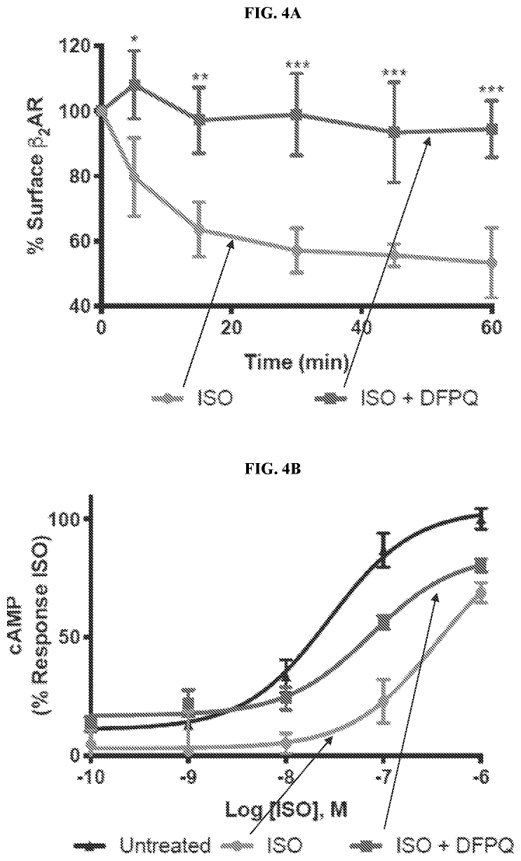

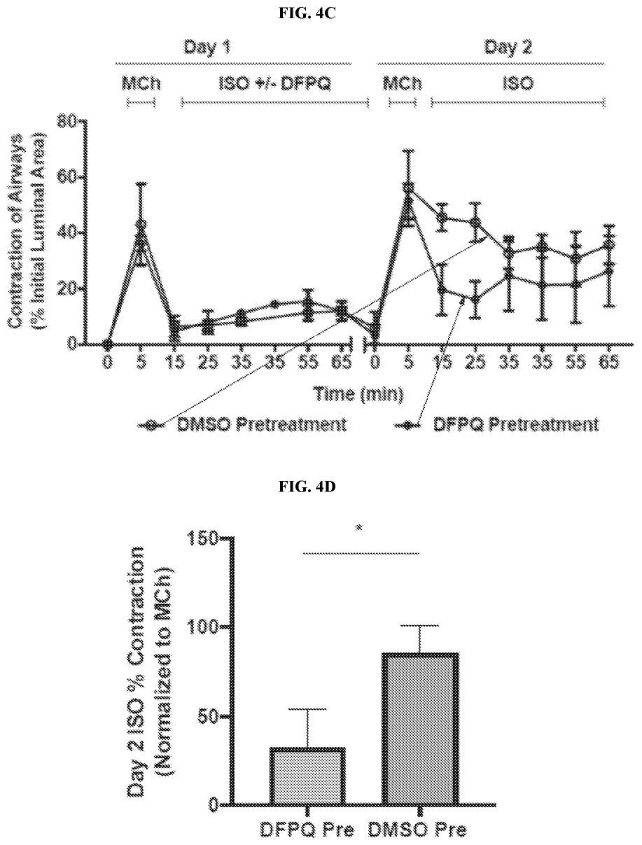

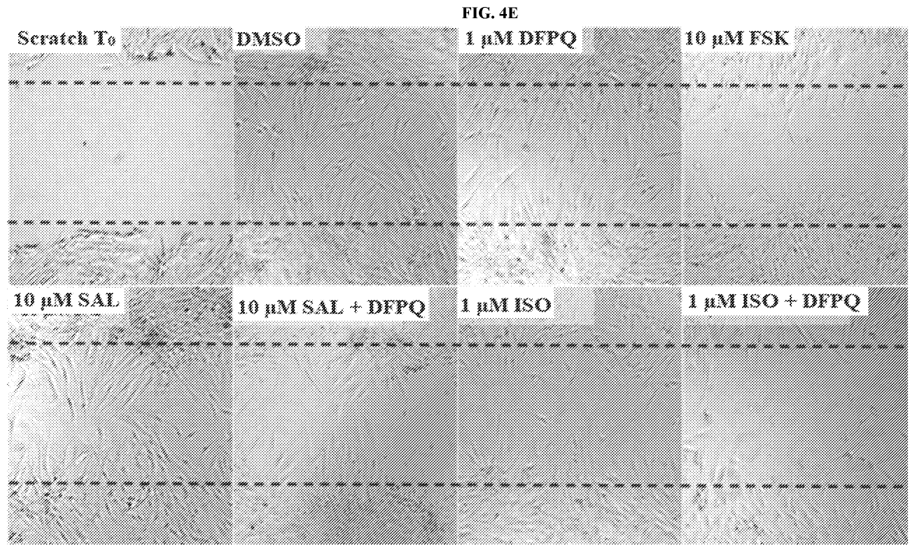

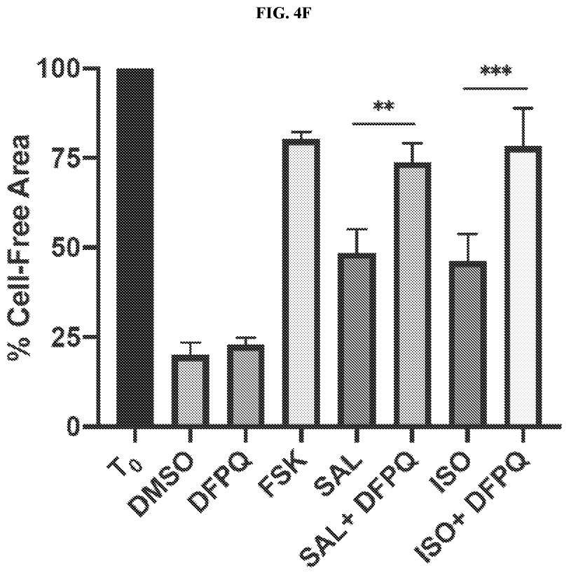

β-arrestin binding to the β₂AR is essential for agonist-promoted internalization of the receptor. To evaluate the ability of DFPQ to modulate β₂AR internalization, cell surface expression of FLAG-tagged β₂AR was measured by ELISA post ISO stimulation. ISO induced a rapid decrease in cell surface expression of the β₂AR sustained over a 60 min time-course while agonist-induced internalization of the receptor was fully inhibited by DFPQ (FIG. 4A).

To evaluate whether DFPQ can inhibit agonist-induced desensitization of the β₂AR, we established an in-cell desensitization assay to monitor cAMP response after sustained agonist treatment. HEK 293 cells were stimulated with ISO for 30 min in the presence or absence of DFPQ, washed, and then re-stimulated with various doses of ISO to generate dose response curves for cAMP production. Relative to a control generated from cells that were not pre-treated with ISO, a 15-fold right-shift in the ISO EC₅₀ was observed (29±6 nM in control vs. 432±140 nM in pre-treated cells) (FIG. 4B). Addition of 10 RM DFPQ during the 30 min ISO pre-treatment largely protected against this desensitization (EC₅₀=74±5 nM). Taken together, these data demonstrate that DFPQ effectively attenuates agonist-induced desensitization and internalization of the β₂AR.

### Example 6: Airway Smooth Muscle Function is Protected from Desensitization by DFPQ Treatment

The pathophysiology of asthma is complex and multifactorial. In order to examine the observed protective effects of DFPQ on β₂AR desensitization in a physiologically relevant system, in-cell desensitization experiments were recapitulated in mouse airway tissue. The muscarinic receptor agonist methacholine was used to contract airway smooth muscle. The contraction was rapidly reversed by treatment with ISO in the presence or absence of DFPQ (FIG. 4C, day 1). The following day, and after chronic (overnight) exposure to ISO, airway tissues were washed, re-challenged with methacholine and again treated with ISO and airway contractility was measured. In this ex vivo model for agonist-induced desensitization of response, ISO mediated reversal of airway contraction was significantly desensitized on day 2 but was largely preserved in the DFPQ pre-treated tissue relative to control (FIG. 4C). DFPQ shows significant protection from desensitization up to 25 minutes post ISO challenge on day 2 (FIG. 4D). This result is consistent with the observed biochemical and cell data, and indicates that DFPQ can effectively block β₂AR desensitization in a complex physiologically relevant system.

The effects of DFPQ on beta-agonist regulation of primary human airway smooth muscle (HASM) cell migration was investigated using a scratch assay. ASM migration is believed to contribute to deleterious airway remodeling that leads to irreversible lung resistance in chronic asthma. The Gₛ-adenylyl cyclase-cAMP-PKA signaling axis is inhibitory to this HASM migration. At 24 hours post scratch, HASM cells incubated with 20 ng/mL platelet derived growth factor (PDGF) (DMSO control) migrated into the scratched area while addition of the adenylyl cyclase activator forskolin (FSK) effectively inhibited migration (FIG. 4E). Cells treated with PDGF plus the β-agonists salmeterol (SAL) or ISO both showed reduced migration relative to PDGF alone, however, this effect was significantly enhanced in the presence of 1 μM DFPQ. These data are quantified in FIG. 4F and demonstrate that DFPQ enhances Gₛ-dependent effects of β-agonists in primary HASM cells. Without wishing to be limited by any theory, these results indicate that in certain embodiments β-arrestin-biased NAMs such as DFPQ can help address an underlying cause of asthma pathogenesis by better inhibiting airway remodeling.

It is now understood that simple on/off models of receptor activation do not capture the full complement of GPCR signal transduction. As a result, single endpoint measurement in drug discovery efforts for GPCR targeted therapeutics necessarily excludes information about receptor signaling or regulation that may be relevant to side-effect profiles for a novel receptor ligand. A single GPCR may couple to multiple G proteins, interact with multiple kinases and arrestins, and have other binding partners that modulate signaling and/or regulate receptor expression. A therapeutic effect may be downstream of a measured endpoint, while harmful side effects may be downstream of other signaling events or protein-protein interactions. Interactions with downstream transducers/effectors of GPCR signaling are coupled to conformational changes in the receptor, and experimental techniques such as nuclear magnetic resonance spectroscopy and hydrogen deuterium exchange in aqueous solution have shown that GPCRs constantly explore conformational space. The concept of biased signaling suggests that a receptor can be stabilized in a conformation that selectively promotes receptor activation toward specific downstream effectors or one that prevents subsequent interactions with regulatory proteins. Thorough profiling of receptor signaling with respect to physiological response provides an avenue toward discovering ligands that promote therapeutic responses and minimize interactions with effectors that promote harmful side effects. The potential value of this approach to discovery has been previously explored for several GPCRs including μ-opioid, cannabinoid and dopamine receptors.

In the work described herein, β₂AR-mediated Gₛ activation and β-arrestin2 interaction were examined. Previous studies have shown that cAMP production through Gₛ is the primary mediator of airway smooth muscle relaxation while GRK phosphorylation and β-arrestin recruitment promote the desensitization of response to β-agonists and the internalization of the β₂AR. β-arrestins have also been implicated in eliciting an inflammatory response in the airway. For these reasons, molecules that bias β₂AR signaling toward Gₛ without promoting β-arrestin recruitment can hold clinical utility for reversing airway constriction in asthma attacks without the side effects currently observed for balanced β-agonists. The effects of test compounds on cAMP production and β-arrestin interaction in the presence of the balanced agonist ISO led to the discovery of DFPQ, a potent and selective β-arrestin-biased NAM for the β₂AR. In the presence of ISO, DFPQ was able to antagonize GRK-mediated phosphorylation and β-arrestin interaction with the β₂AR without inhibiting cAMP production. Moreover, when examined in functional assays, the effects of DFPQ interaction on the β₂AR demonstrated the hallmarks of allostery.

The extracellular domains of GPCRs have gained appreciation as novel druggable allosteric sites. Rearrangements in the extracellular loops and transmembrane domains can create a secondary binding pocket that has been referred to as the “extracellular vestibule,” and these domains have roles in ligand recognition, receptor specificity, and signaling properties for a variety of GPCRs. The interaction of the M2 muscarinic acetylcholine receptor (M2AChR) with the selective positive allosteric modulator, LY2119620, is the prototypical example of this type of small molecule modulator. Structural coupling of the extracellular vestibule, the orthosteric binding pocket, and the intracellular surface of the receptor likely account for the ability of LY2119620 to affect the affinity and efficacy of orthosteric ligands. While the DFPQ binding site on the β₂AR is not known, and without wishing to be limited by any theory, allosteric modulators of the β₂AR targeting a homologous site may similarly affect the interactions of extracellular domains with transmembrane domains, thereby altering the signaling properties of known β-agonists. The results of this study indicate that DFPQ can modify the agonist-induced active state of the receptor such that the β₂AR preferentially interacts with Gₛ compared with GRKs and β-arrestins.

β-agonists that are currently prescribed for asthma come with “black box” warnings from the US Food and Drug Administration, and studies have shown that long-term use of some of these drugs increases the risk of hospitalization, life-threatening exacerbation, and death. This is due in part to GRK and β-arrestin interactions that induce desensitization and internalization of the β₂AR and promote inflammatory responses. β-arrestins are critical in the pathogenesis of asthma with both inflammatory and physiological effects. Various studies utilizing in vivo and in vitro models of pathological asthmatic inflammatory response have shown that β-arrestin2 knockout is protective from mucin production, airway hyper-responsiveness and immune cell infiltration. Thus, compounds that inhibit β-arrestin interaction with the β₂AR like DFPQ can hold a clinical advantage by mitigating deleterious arrestin-dependent effects. In this study, DFPQ was shown to inhibit agonist-induced internalization of the β₂AR and to protect against agonist-induced desensitization in both cell and ex vivo tissue models. Furthermore, DFPQ enhances the PKA dependent inhibition of HASM migration in primary cells, which may address airway remodeling in the pathogenesis of asthma. For these reasons, DFPQ constitutes an improved class of asthma therapeutics. Additionally, DFPQ can be used to better describe the molecular mechanisms of biased signaling through the β₂AR and the diverse signaling profiles propagated by β₂AR ligands.

Table 2 and Table 3 illustrate various compounds contemplated within the disclosure and selected biological data of compound comprising different R₁, R₂, and/or R₃ substituents. The structure activity relationship of quinazoline derivatives on β₂AR-promoted activation of cAMP production and β-arrestin2 binding is shown as functional group substitutions effect on PathHunter™ IC₅₀ values. The fold bias is denoted as the GloSensor™ IC₅₀:PathHunter™ IC₅₀ ratio.

TABLE 2
Assay Results for Selected Compounds

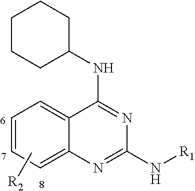

| | | | |
|---|---|---|---|
| R1 Substituent | R₂ Substituent | IC₅₀ Arrestin (μM) | *Arrestin fold bias |

| | | | |
|---|---|---|---|
| ethylpropylether | N/A^{§} | 12 ± 1 | No |
| cyclohexyl | N/A | 4 ± 1 | >10 |
| m-trifluoromethylphenyl | N/A | 3 ± 1 | ~3 |
| m-chlorobenzyl | N/A | 21 ± 2 | No |
| p-methylbenzyl | N/A | 8 ± 2 | ~4 |
| m,p-dimethoxyphenyl | N/A | 14 ± 3 | >7 |
| p-chlorophenyl | N/A | 0.8 ± 0.2 | >100 |
| m,p-dichlorophenyl | N/A | 3.0 ± 0.7 | >100 |
| p-fluorophenyl | N/A | 0.23 ± 0.07 | >200 |
| m-fluorophenyl | N/A | 0.4 ± 0.1 | >200 |
| m-difluorophenyl | N/A | 0.20 ± 0.05 | >200 |
| m-chlorophenyl | N/A | 6.0 ± 0.8 | >10 |
| m-chloro-p-fluorophenyl | N/A | 3 ± 1 | ~9 |
| phenyl | N/A | 3.0 ± 0.7 | >100 |
| m-dichlorophenyl | 6-methyl | 3.0 ± 0.5 | >20 |
| m-dichlorophenyl | 7-methyl | 10 ± 3 | >10 |
| m-dichlorophenyl | 8-methyl | >100 | No |
| m-dichlorophenyl | 6-chloro | 8 ± 2 | >20 |
| m,p-difluorophenyl | 6-chloro | 0.12 ± 0.05 | >400 |
| m,p-difluorophenyl | 6-fluoro | 0.50 ± 0.15 | >30 |
| m,p-difluorophenyl | 6-bromo | 0.03 ± 0.02 | >900 |
| m,p-difluorophenyl | 6-methyl | 0.36 ± 0.18 | >45 |
| m,p-difluorophenyl | 6-trifluoromethyl | 0.07 ± 0.04 | >1000 |

*GloSensor ™ IC₅₀: PathHunter ™ IC₅₀
^{§}N/A indicates that all R₂ are H

TABLE 3
Assay Results for Selected Compounds

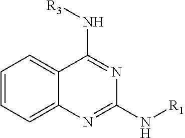

| R₁ Substituent | R₃ Substituent | IC₅₀ Arrestin (μM) | *Arrestin fold bias |
|---|---|---|---|
| m-dichlorophenyl | H | 10 ± 2 | No |
| m-dichlorophenyl | ethyl | 13 ± 2 | No |
| m-dichlorophenyl | pentyl | 23 ± 5 | ~3 |
| m-dichlorophenyl | cyclopentyl | 9 ± 3 | ~2 |
| m-dichlorophenyl | 3-methylcyclohexyl | 8 ± 5 | >10 |
| m-dichlorophenyl | cycloheptyl | 5 ± 2 | >20 |
| m-dichlorophenyl | propylamine | 25 ± 2 | No |
| m-dichlorophenyl | dimethylpropylamine | 26 ± 3 | >3 |
| m-dichlorophenyl | piperidine | 22 ± 3 | >6 |
| m-chloro-p- | piperidine | 7 ± 2 | ~9 |
| fluorophenyl | | | |
| m-dichlorophenyl | p- | 18 ± 2 | No |
| | hydroxyethylphenyl | | |
| m-dichlorophenyl | benzyl | 30 ± 4 | No |
| m-dichlorophenyl | phenethyl | 7 ± 2 | ~3 |

*GloSensor ™ IC₅₀: PathHunter ™ IC₅₀

Sequence Listing

SEQ ID NO: 1 (β2-adrenergic receptor)
MGQPGNGSAFLLAPNGSHAPDHDVTQERDEVWVVGMGIVMSLIVLAIVEG
NVLVITAIAKFERLQTVTNYFITSLACADLVMGLAVVPFGAAHILMKMWT
FGNFWCEFWTSIDVLCVTASIETLCVIAVDRYFAITSPFKYQSLLTKNKA
RVIILMVWIVSGLTSFLPIQMHWYRATHQEAINCYANETCCDFFTNQAYA
IASSIVSFYVPLVIMVFVYSRVFQEAKRQLQKIDKSEGRFHVONLSQVEQ
DGRTGHGLRRSSKFCLKEHKALKTLGIIMGTFTLCWLPFFIVNIVHVIQD
NLIRKEVYILLNWIGYVNSGFNPLIYCRSPDFRIAFQELLCLRRSSLKAY
GNGYSSNGNTGEQSGYHVEQEKENKLLCEDLPGTEDFVGHQGTVPSDNID
SQGRNCSTNDSLL

### Enumerated Embodiments

The following enumerated embodiments are provided, the numbering of which is not to be construed as designating levels of importance.

Embodiment 1 provides a method of treating airway disease in a subject, the method comprising at least one of the following:
(i) administering to the subject a therapeutically effective amount of a compound of formula (1), or a pharmaceutically acceptable salt, solvate, isotopologue, stereoisomer, tautomer, and/or any mixture thereof:

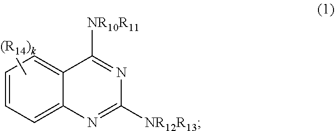

wherein:
R₁₀ and R₁₁ are each independently selected from the group consisting of H and optionally substituted C₃-C₈ cycloalkyl;
R₁₂ and R₁₃ are each independently selected from the group consisting of H; C₁-C₆ alkyl; C₁-C₆ alkoxy; optionally substituted C₃-C₈ cycloalkyl; —(CH₂)ₙO(CH₂)ₘCH₃; optionally monosubstituted phenyl wherein the optional substituent is selected from the group consisting of F, Br, NO₂, CF₃, —S(═O)₂CH₃, —O(CH₂)ₚOCH₃, and

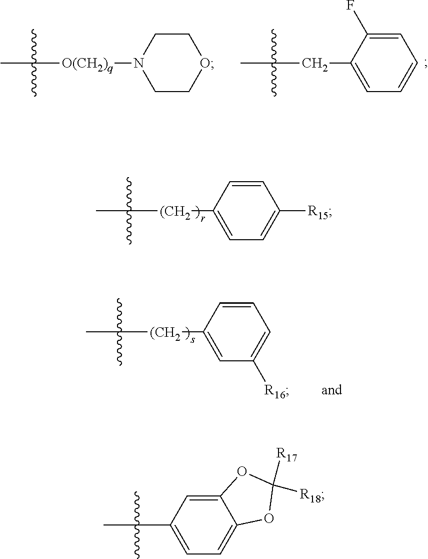

or
   R₁₂ and R₁₃ combine with the nitrogen to which they are attached to form

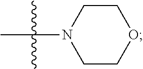 [SMILES: *N1CCOCC1]

each occurrence of R₁₄ is independently selected from the group consisting of H, C₁-C₆ alkyl, F, Br, Cl, and I;
R₁₅, R₁₇, and R₁₈ are each independently selected from the group consisting of C₁-C₆ alkyl, F, Br, Cl, and I;
R₁₆ is selected from the group consisting of F, Br, Cl, I, and —C(R₁₉)₃;
R₁₉ is selected from the group consisting of F, Br, Cl, and I;
k is 4;
m, n, p, and q are each independently selected from the group consisting of 1, 2, and 3; and
r and s are each independently selected from the group consisting of 0 and 1;
(ii) administering to the subject a therapeutically effective amount of a compound of formula (2), or a pharmaceutically acceptable salt, solvate, isotopologue, stereoisomer, tautomer, and/or any mixture thereof:

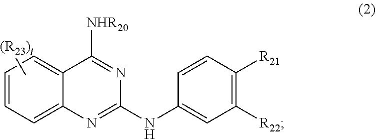

wherein:
R₂₀ is selected from the group consisting of C₃-C₈ cycloalkyl and C₄-C₇ heterocycloalkyl; R₂₁ and R₂₂ are each independently selected from the group consisting of F, Cl, Br, I,
C₁-C₆ alkoxy, and —SC(R₂₄)₃, with the proviso that R₂₁ is C₁-C₆ alkoxy if and only if R₂₂ is C₁-C₆ alkoxy;
each occurrence R₂₃ is independently selected from the group consisting of H, C₁-C₆ alkyl, —C(R₂₅)₃, F, Cl, Br, and I;
each occurrence of R₂₄ and R₂₅ is independently selected from the group consisting of
F, Cl, Br, and I; and
t is 4; or
(iii) administering to the subject a therapeutically effective amount of a compound of formula (3), or a pharmaceutically acceptable salt, solvate, isotopologue, stereoisomer, tautomer, and/or any mixture thereof:

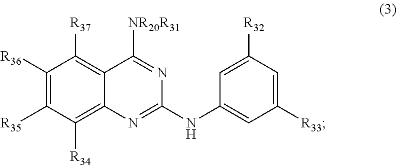

wherein:
R₃₀ and R₃₁ are each independently selected from the group consisting of: H; C₁-C₆ linear alkyl optionally substituted with at least one substituent selected from the group consisting of —NR₃₈R₃₉, C₃-C₈ cycloalkyl, phenyl, and OH; C₃-C₁₂ cycloalkyl optionally substituted with at least one substituent selected from the group consisting of C₁-C₆ alkyl, C═O, and —NR₃₈R₃₉, wherein the —NR₃₈R₃₉ is optionally substituted with C₄-C₇ heterocycloalkyl; C₄-C₇ heterocycloalkyl optionally substituted with at least one substituent selected from the group consisting of phenyl, benzyl, CH₂CH₂OCH₃, and C(═O)R₄₁; phenyl substituted with one or more substituents selected from the group consisting of F, Br, I, OH, C₁-C₆ alkyl, C₁-C₆ alkoxy, —N(R₄₀)₂, and —C(═O)R₄₁;

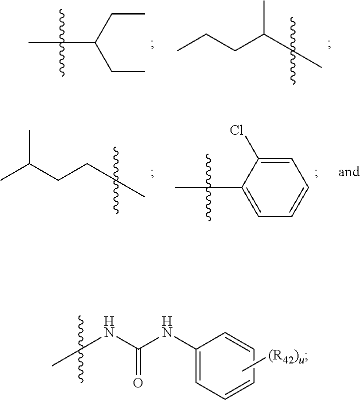

R₃₂ and R₃₃ are each independently selected from the group consisting of F, Cl, Br, and I; R₃₄ and R₃₆ are each independently selected from the group consisting of H, C₁-C₆ alkyl, C₁-C₆ alkoxy, F, Cl, Br, and I;
R₃₅ and R₃₇ are each independently selected from the group consisting of H and C₁-C₆ alkyl;
R₃₈ and R₃₉ are each independently selected from the group consisting of H, C₁-C₆ alkyl, and C₆-C₁₂ aryl;
each R₄₀ is selected from the group consisting of H and C₁-C₆ linear alkyl;
R₄₁ is selected from the group consisting of H, C₁-C₆ alkyl, and C₁-C₆ alkoxy;
R₄₂ is selected from the group consisting of F, Cl, Br, and I; and
u is 1, 2, 3, 4, or 5.

Embodiment 2 provides the method of Embodiment 1, wherein the compound comprises a compound of formula (1) wherein one of the following applies:
(i) R₁₀ and R₁₁ are each H; or
(ii) R₁₀ is H and R₁₁ is selected from the group consisting of

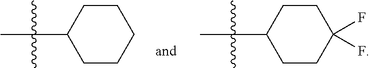 [SMILES: *C1CCCCC1 ; *C1CCC(F)(F)CC1]

Embodiment 3 provides the method of Embodiment 1 or 2, wherein the compound comprises a compound of formula (1) wherein one of the following applies:
(i) R₁₂ and R₁₃ are each H;
(ii) R₁₂ and R₁₃ are each CH₂CH₃;
(iii) R₁₂ is CH₃ and R₁₃ is phenyl;
(iii) R₁₂ is H and R₁₃ is selected from the group consisting of

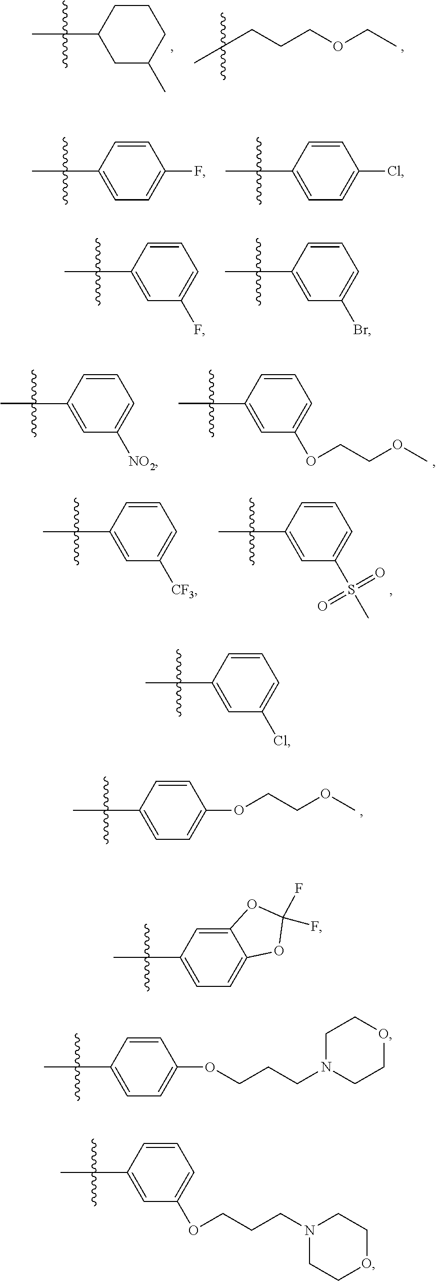

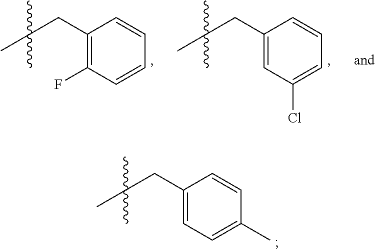

or
(iv) R₁₂ and R₁₃ combine with the nitrogen to which they are attached to form

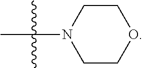 [SMILES: *N1CCOCC1]

Embodiment 4 provides the method of any one of Embodiments 1-3, wherein the compound comprises a compound of formula (1) wherein one of the following applies:
(i) each R₁₄ is independently H; or
(ii) three R₁₄ are H and one R₁₄ is selected from the group consisting of Cl and F.

Embodiment 5 provides the method of Embodiment 1, wherein the compound comprises a compound of formula (1) selected from the group consisting of:

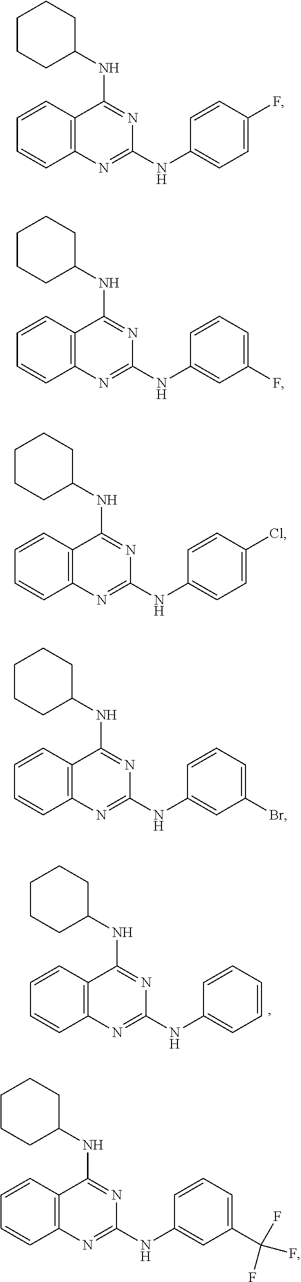 [SMILES: Fc1ccc(Nc2nc(NC3CCCCC3)c3ccccc3n2)cc1 ; Fc1cccc(Nc2nc(NC3CCCCC3)c3ccccc3n2)c1 ; Clc1ccc(Nc2nc(NC3CCCCC3)c3ccccc3n2)cc1 ; Brc1cccc(Nc2nc(NC3CCCCC3)c3ccccc3n2)c1 ; c1ccc(Nc2nc(NC3CCCCC3)c3ccccc3n2)cc1 ; FC(F)(F)c1cccc(Nc2nc(NC3CCCCC3)c3ccccc3n2)c1]

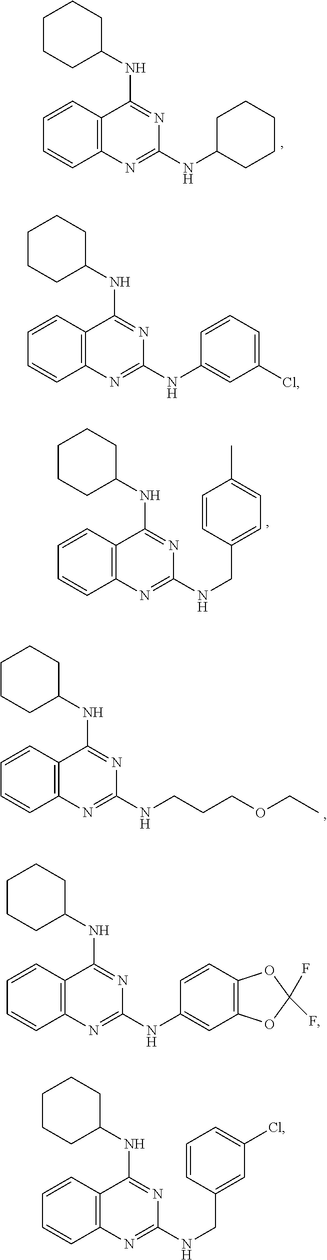 [SMILES: c1ccc2c(NC3CCCCC3)nc(NC3CCCCC3)nc2c1 ; Clc1cccc(Nc2nc(NC3CCCCC3)c3ccccc3n2)c1 ; Cc1ccc(CNc2nc(NC3CCCCC3)c3ccccc3n2)cc1 ; CCOCCCNc1nc(NC2CCCCC2)c2ccccc2n1 ; FC1(F)Oc2ccc(Nc3nc(NC4CCCCC4)c4ccccc4n3)cc2O1 ; Clc1cccc(CNc2nc(NC3CCCCC3)c3ccccc3n2)c1]

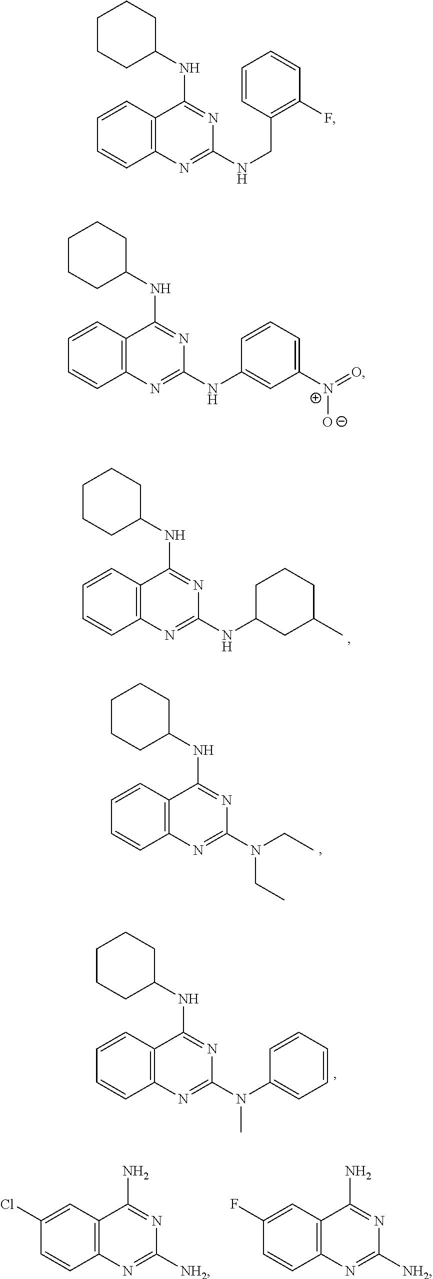

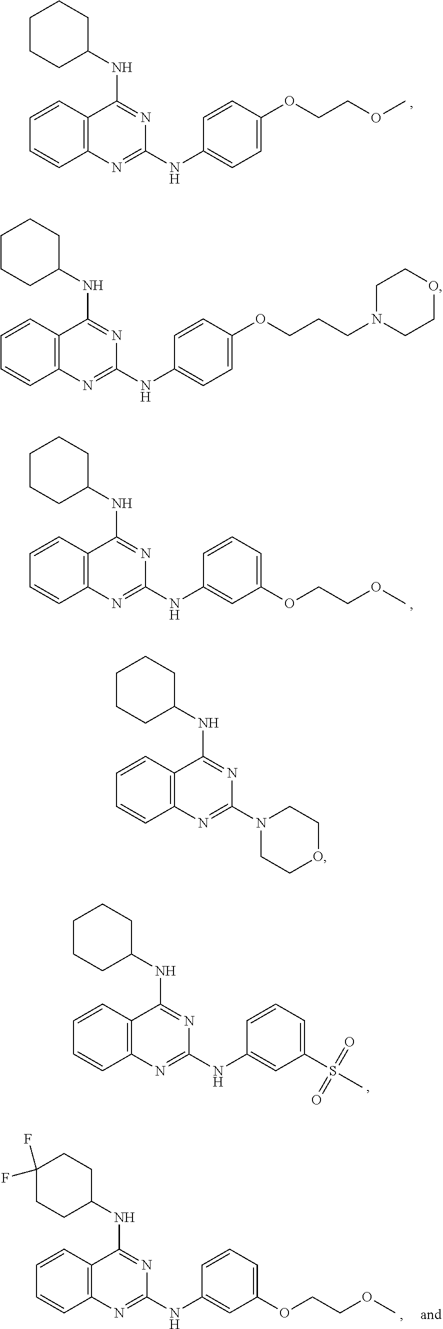 [SMILES: COCCOc1ccc(Nc2nc(NC3CCCCC3)c3ccccc3n2)cc1 ; c1ccc2c(NC3CCCCC3)nc(Nc3ccc(OCCCN4CCOCC4)cc3)nc2c1 ; COCCOc1cccc(Nc2nc(NC3CCCCC3)c3ccccc3n2)c1 ; c1ccc2c(NC3CCCCC3)nc(N3CCOCC3)nc2c1 ; CS(=O)(=O)c1cccc(Nc2nc(NC3CCCCC3)c3ccccc3n2)c1 ; COCCOc1cccc(Nc2nc(NC3CCC(F)(F)CC3)c3ccccc3n2)c1]

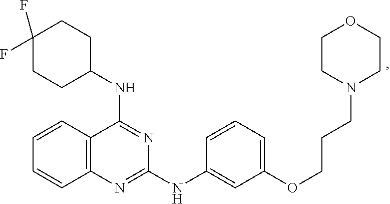 [SMILES: FC1(F)CCC(Nc2nc(Nc3cccc(OCCCN4CCOCC4)c3)nc3ccccc23)CC1]

and combinations thereof.

Embodiment 6 provides the method of Embodiment 1, wherein the compound comprises a compound of formula (2) wherein R₂₀ is selected from the group consisting of cyclohexane,

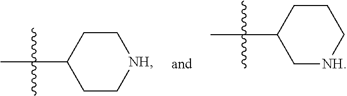 [SMILES: *C1CCNCC1 ; *C1CCCNC1]

Embodiment 7 provides the method of Embodiment 1 or 6, wherein the compound comprises a compound of formula (2) wherein one of the following applies:
(i) each of R₂₁ is F and R₂₂ is F;
(ii) each of R₂₁ is C₁ and R₂₂ is Cl;
(iii) each of R₂₁ is OCH₃ and R₂₂ is OCH₃;
(iv) R₂₁ is F and R₂₂ is Cl;
(v) R₂₁ is C₁ and R₂₂ is F;
(vi) R₂₁ is C₁ and R₂₂ is SCF₃; or
(vii) R₂₁ is SCF₃ and R₂₂ is Cl.

Embodiment 8 provides the method of any one of Embodiments 1 and 6-7, wherein the compound comprises a compound of formula (2) wherein one of the following applies:
(i) each of R₂₃ is H; or
(ii) three of R₂₃ are H and one R₂₃ is selected from the group consisting of F, Cl, Br, CH₃, and CF₃.

Embodiment 9 provides the method of Embodiment 1 or 6, wherein the compound comprises a compound of formula (2) selected from the group consisting of:

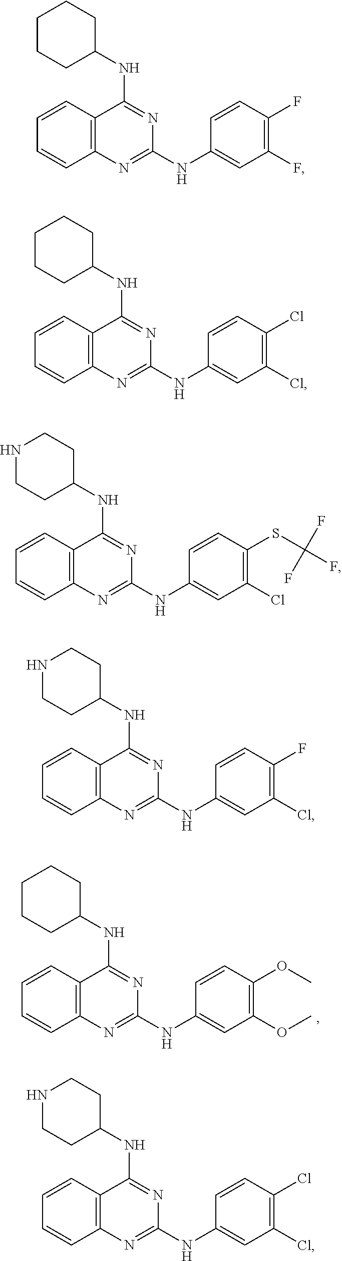 [SMILES: Fc1ccc(Nc2nc(NC3CCCCC3)c3ccccc3n2)cc1F ; Clc1ccc(Nc2nc(NC3CCCCC3)c3ccccc3n2)cc1Cl ; FC(F)(F)Sc1ccc(Nc2nc(NC3CCNCC3)c3ccccc3n2)cc1Cl ; Fc1ccc(Nc2nc(NC3CCNCC3)c3ccccc3n2)cc1Cl ; COc1ccc(Nc2nc(NC3CCCCC3)c3ccccc3n2)cc1OC ; Clc1ccc(Nc2nc(NC3CCNCC3)c3ccccc3n2)cc1Cl]

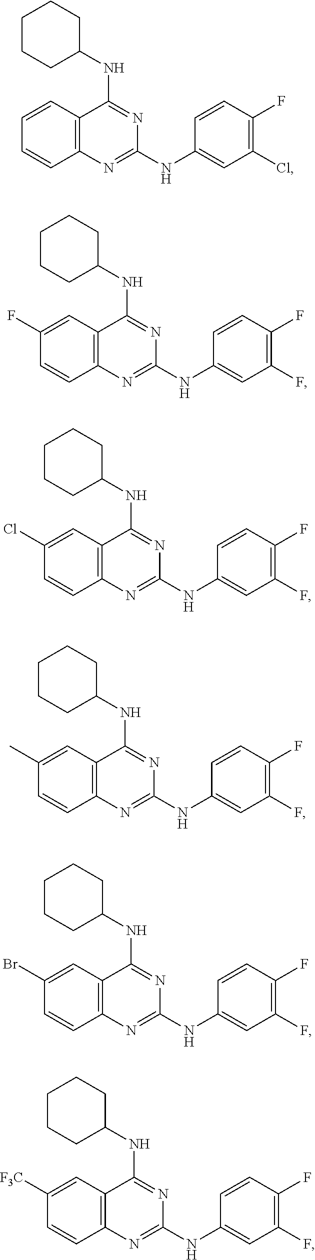 [SMILES: Fc1ccc(Nc2nc(NC3CCCCC3)c3ccccc3n2)cc1Cl ; Fc1ccc2nc(Nc3ccc(F)c(F)c3)nc(NC3CCCCC3)c2c1 ; Fc1ccc(Nc2nc(NC3CCCCC3)c3cc(Cl)ccc3n2)cc1F ; Cc1ccc2nc(Nc3ccc(F)c(F)c3)nc(NC3CCCCC3)c2c1 ; Fc1ccc(Nc2nc(NC3CCCCC3)c3cc(Br)ccc3n2)cc1F ; Fc1ccc(Nc2nc(NC3CCCCC3)c3cc(C(F)(F)F)ccc3n2)cc1F]

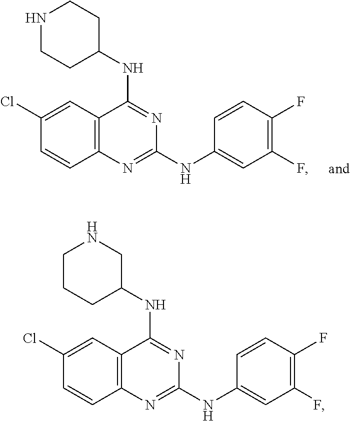 [SMILES: Fc1ccc(Nc2nc(NC3CCNCC3)c3cc(Cl)ccc3n2)cc1F ; Fc1ccc(Nc2nc(NC3CCCNC3)c3cc(Cl)ccc3n2)cc1F]

and combinations thereof.

Embodiment 10 provides the method of Embodiment 1, wherein the compound comprises a compound of formula (3) wherein one of the following applies:
(i) R₃₀ and R₃₁ are each H;
(ii) R₃₀ and R₃₁ are each —CH₂CH₃; or
(iii) R₃₀ is H and R₃₁ is selected from the group consisting of —CH₂CH₃, —(CH₂)₄CH₃, —(CH₂)₂₀H, —(CH₂)phenyl, —(CH₂)₂phenyl, —(CH₂)cyclohexane, —(CH₂)₂cyclohexane, —(CH₂)₃NH₂, —(CH₂)₃N(CH₃)₂,

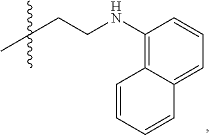 [SMILES: *CCNc1cccc2ccccc12]

cyclopentane, cyclohexane, cycloheptane, adamantyl,

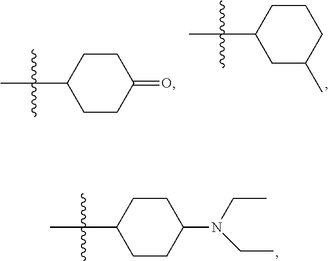

piperidine,

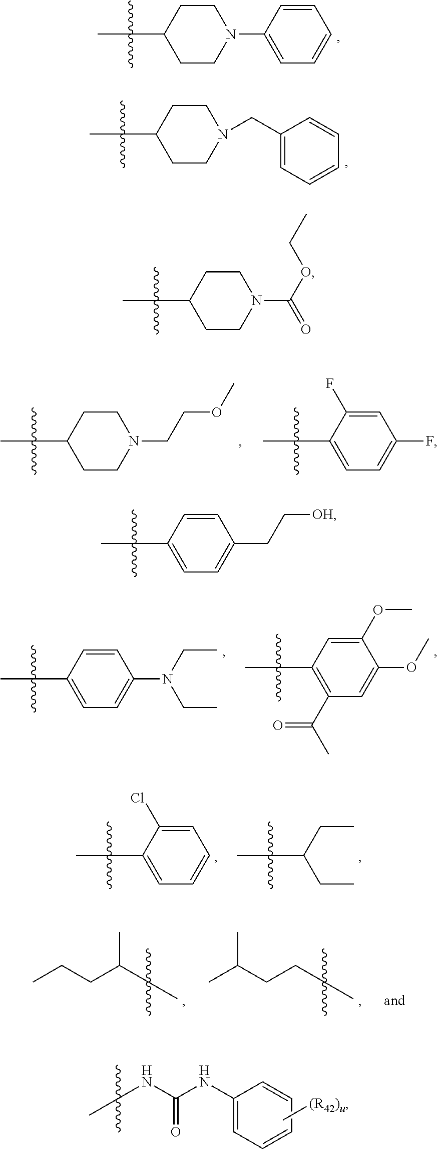

wherein R₄₂ is F and u is 1.

Embodiment 11 provides the method of Embodiment 1 or 10, wherein the compound comprises a compound of formula (3) wherein one of the following applies:
(i) R₃₂ is Cl and R₃₃ is Cl;
(ii) R₃₂ is F and R₃₃ is F; or
(iii) R₃₂ is F and R₃₃ is I.

Embodiment 12 provides the method of any one of Embodiments 1 and 10-11, wherein the compound comprises a compound of formula (3) wherein at least one of the following applies:
(i) R₃₄ is selected from the group consisting of H and CH₃;
(ii) R₃₅ is selected from the group consisting of H and CH₃;
(iii) R₃₆ is selected from the group consisting of H, CH₃, F, and Cl; and
(iv) R₃₇ is H.

Embodiment 13 provides the method of Embodiment 1, wherein the compound comprises a compound of formula (3) selected from the group consisting of:

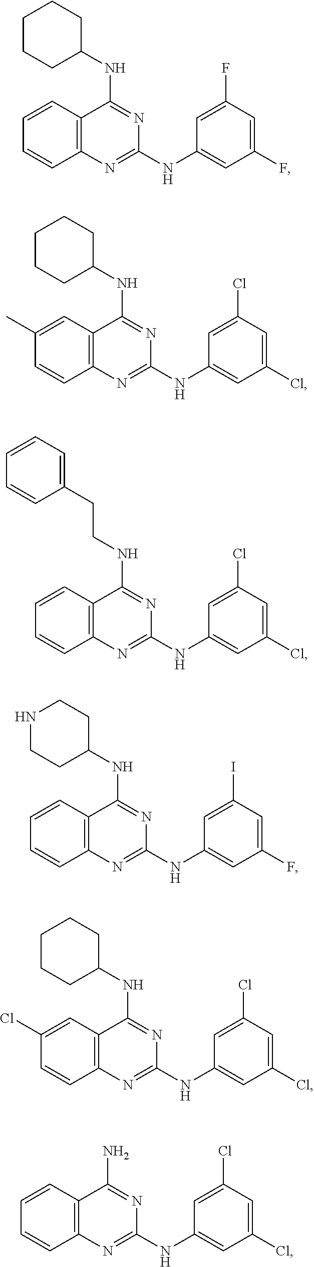 [SMILES: Fc1cc(F)cc(Nc2nc(NC3CCCCC3)c3ccccc3n2)c1 ; Cc1ccc2nc(Nc3cc(Cl)cc(Cl)c3)nc(NC3CCCCC3)c2c1 ; Clc1cc(Cl)cc(Nc2nc(NCCc3ccccc3)c3ccccc3n2)c1 ; Fc1cc(I)cc(Nc2nc(NC3CCNCC3)c3ccccc3n2)c1 ; Clc1cc(Cl)cc(Nc2nc(NC3CCCCC3)c3cc(Cl)ccc3n2)c1 ; Nc1nc(Nc2cc(Cl)cc(Cl)c2)nc2ccccc12]

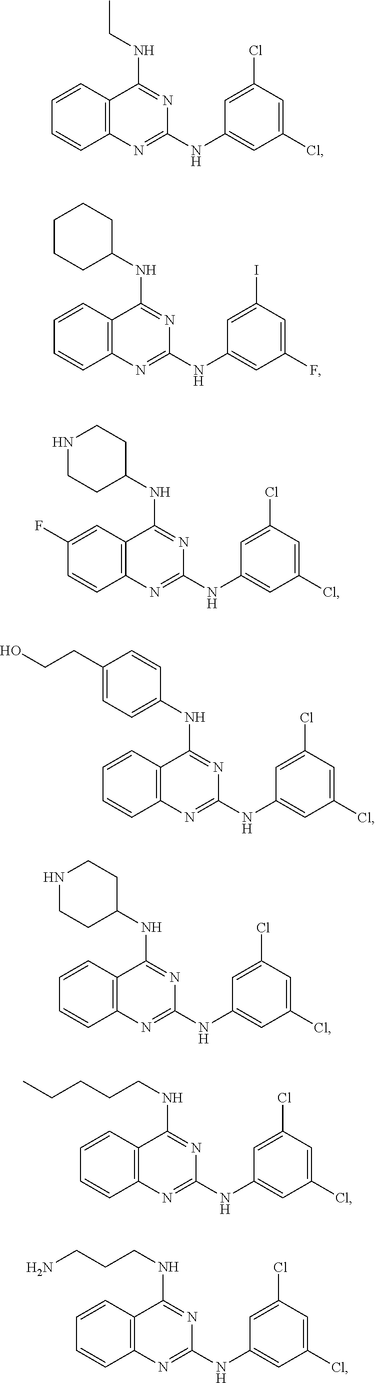

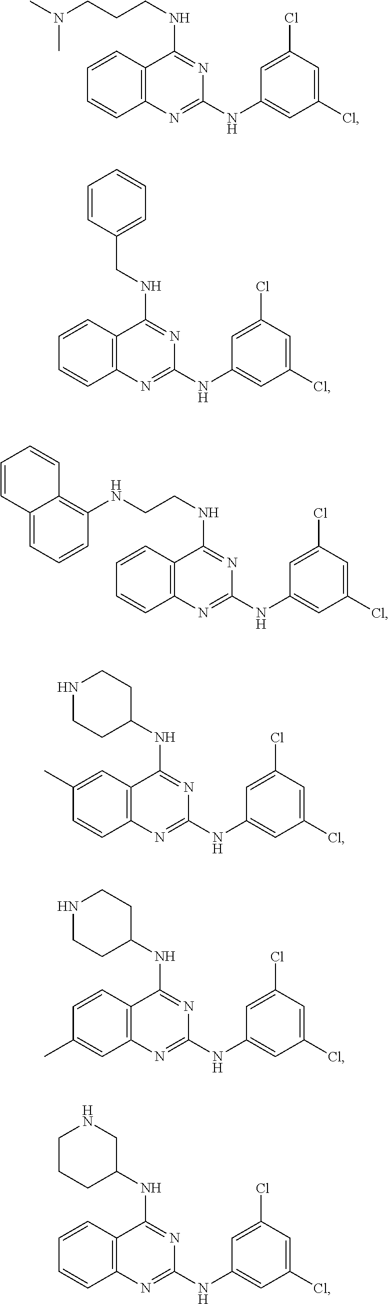 [SMILES: CN(C)CCCNc1nc(Nc2cc(Cl)cc(Cl)c2)nc2ccccc12 ; Clc1cc(Cl)cc(Nc2nc(NCc3ccccc3)c3ccccc3n2)c1 ; Clc1cc(Cl)cc(Nc2nc(NCCNc3cccc4ccccc34)c3ccccc3n2)c1 ; Cc1ccc2nc(Nc3cc(Cl)cc(Cl)c3)nc(NC3CCNCC3)c2c1 ; Cc1ccc2c(NC3CCNCC3)nc(Nc3cc(Cl)cc(Cl)c3)nc2c1 ; Clc1cc(Cl)cc(Nc2nc(NC3CCCNC3)c3ccccc3n2)c1]

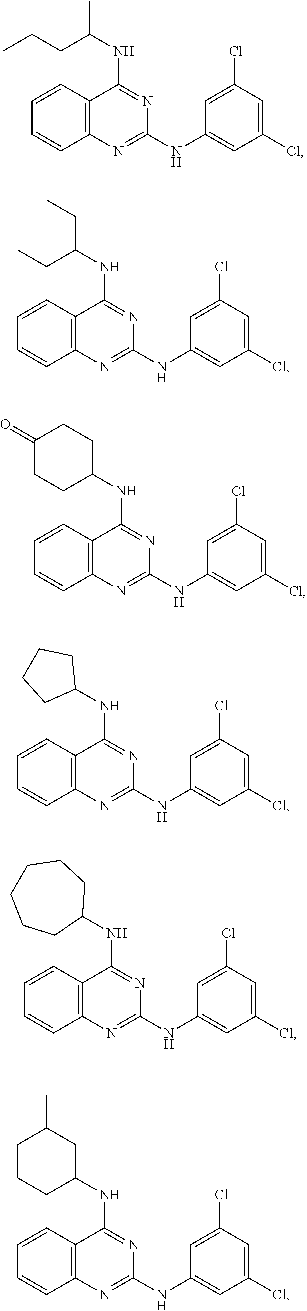 [SMILES: CCCC(C)Nc1nc(Nc2cc(Cl)cc(Cl)c2)nc2ccccc12 ; CCC(CC)Nc1nc(Nc2cc(Cl)cc(Cl)c2)nc2ccccc12 ; O=C1CCC(Nc2nc(Nc3cc(Cl)cc(Cl)c3)nc3ccccc23)CC1 ; Clc1cc(Cl)cc(Nc2nc(NC3CCCC3)c3ccccc3n2)c1 ; Clc1cc(Cl)cc(Nc2nc(NC3CCCCCC3)c3ccccc3n2)c1 ; CC1CCCC(Nc2nc(Nc3cc(Cl)cc(Cl)c3)nc3ccccc23)C1]

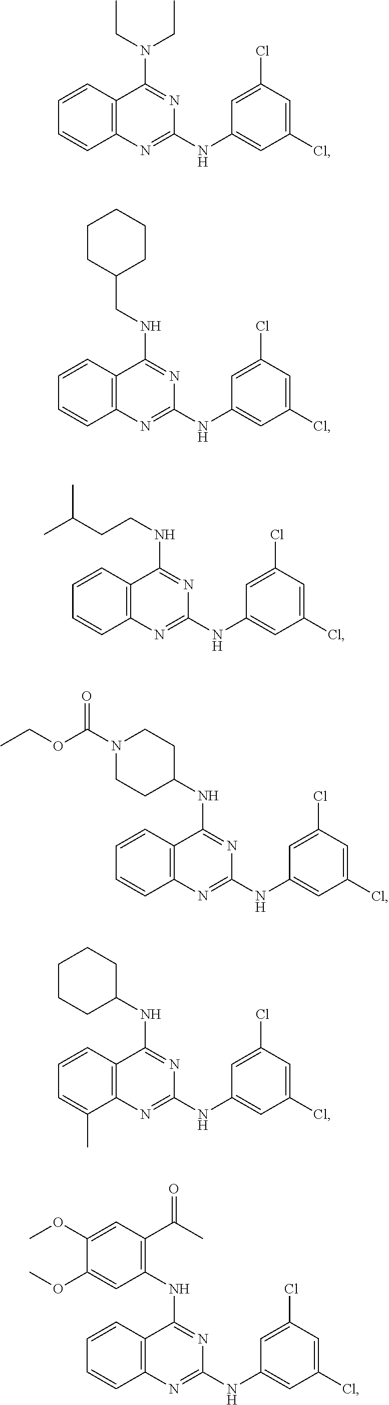 [SMILES: CCN(CC)c1nc(Nc2cc(Cl)cc(Cl)c2)nc2ccccc12 ; Clc1cc(Cl)cc(Nc2nc(NCC3CCCCC3)c3ccccc3n2)c1 ; CC(C)CCNc1nc(Nc2cc(Cl)cc(Cl)c2)nc2ccccc12 ; CCOC(=O)N1CCC(Nc2nc(Nc3cc(Cl)cc(Cl)c3)nc3ccccc23)CC1 ; Cc1cccc2c(NC3CCCCC3)nc(Nc3cc(Cl)cc(Cl)c3)nc12 ; COc1cc(Nc2nc(Nc3cc(Cl)cc(Cl)c3)nc3ccccc23)c(C(C)=O)cc1OC]

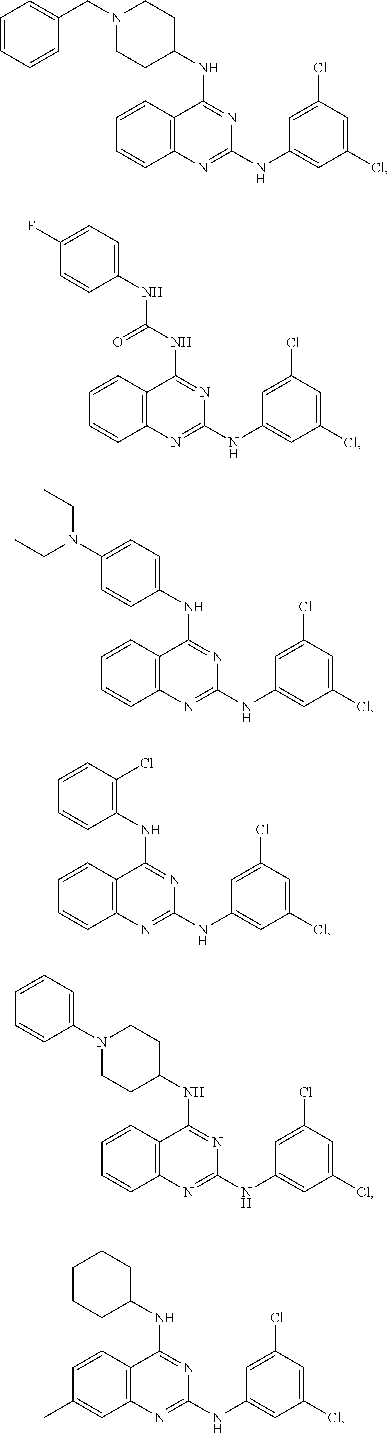 [SMILES: Clc1cc(Cl)cc(Nc2nc(NC3CCN(Cc4ccccc4)CC3)c3ccccc3n2)c1 ; O=C(Nc1ccc(F)cc1)Nc1nc(Nc2cc(Cl)cc(Cl)c2)nc2ccccc12 ; CCN(CC)c1ccc(Nc2nc(Nc3cc(Cl)cc(Cl)c3)nc3ccccc23)cc1 ; Clc1cc(Cl)cc(Nc2nc(Nc3ccccc3Cl)c3ccccc3n2)c1 ; Clc1cc(Cl)cc(Nc2nc(NC3CCN(c4ccccc4)CC3)c3ccccc3n2)c1 ; Cc1ccc2c(NC3CCCCC3)nc(Nc3cc(Cl)cc(Cl)c3)nc2c1]

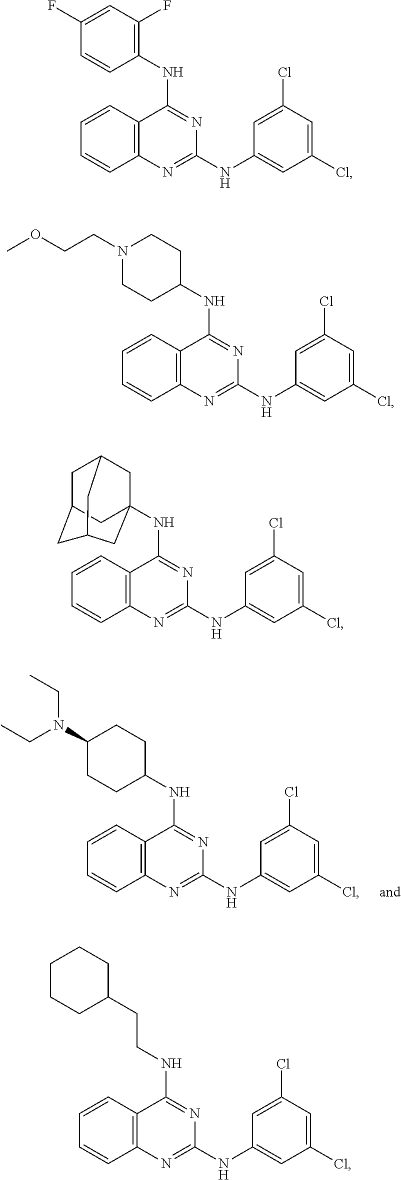

and combinations thereof.

Embodiment 14 provides the method of any one of Embodiments 1-13, wherein the airway disease is asthma or chronic obstructive pulmonary disease.

Embodiment 15 provides the method of any one of Embodiments 1-14, wherein the compound is administered to the subject through a route comprising nasal, inhalational, intratracheal, intrapulmonary, intrabronchial, topical, oral, buccal, rectal, pleural, peritoneal, vaginal, intramuscular, subcutaneous, transdermal, epidural, otic, intraocular, intrathecal, or intravenous.

Embodiment 16 provides the method of any one of Embodiments 1-15, wherein the compound is formulated in a pharmaceutical composition.

Embodiment 17 provides the method of any one of Embodiments 1-16, wherein the subject is a mammal.

Embodiment 18 provides the method of any one of Embodiments 1-17, wherein the subject is a human.

Embodiment 19 provides a compound of formula (1a), or a salt, solvate, isotopologue, stereoisomer, tautomer, and/or any mixture thereof:

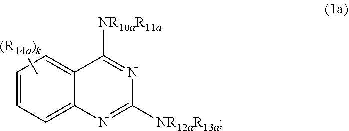

wherein:
R₁₀ₐ and R₁₁ₐ are each independently selected from the group consisting of H and optionally substituted C₃-C₈ cycloalkyl;
R₁₂ₐ is H;
R₁₃ₐ is a monosubstituted phenyl wherein the substituent is selected from —S(═O)₂CH₃, —(CH₂)ₙO(CH₂)ₘCH₃, and

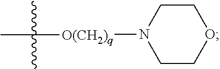

each occurrence of R₁₄ₐ is H;
k is 4; and
m and n are each independently 1, 2, or 3;
   with the proviso that, if R₁₃ₐ is phenyl monosubstituted with —(CH₂)ₙO(CH₂)ₘCH₃, then R₁₁ₐ is substituted C₃-C₈ cycloalkyl.

Embodiment 20 provides the compound of Embodiment 19, wherein at least one of the following applies:
(i) R₁₀ₐ is H and Rim is selected from the group consisting of

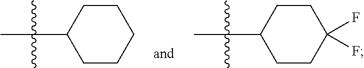 [SMILES: *C1CCCCC1 ; *C1CCC(F)(F)CC1]

(ii) R₁₃ₐ is selected from the group consisting of

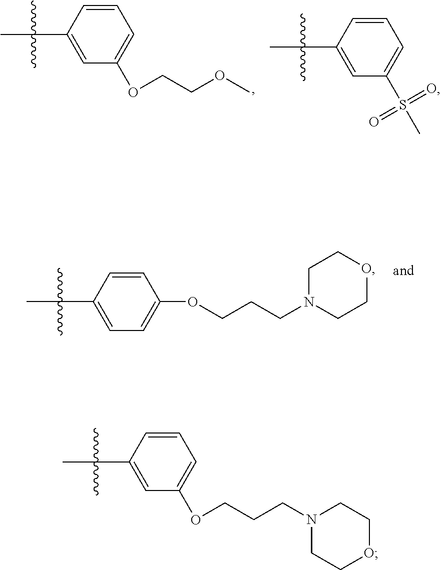 [SMILES: *c1cccc(OCCOC)c1 ; *c1cccc(S(C)(=O)=O)c1 ; *c1ccc(OCCCN2CCOCC2)cc1 ; *c1cccc(OCCCN2CCOCC2)c1]

and
(iii) R₁₃ₐ is phenyl monosubstituted with —(CH₂)ₙO(CH₂)ₘCH₃, and R₁₁ₐ is

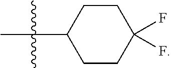 [SMILES: *C1CCC(F)(F)CC1]

Embodiment 21 provides the compound of Embodiment 19, wherein the compound is selected from the group consisting of:

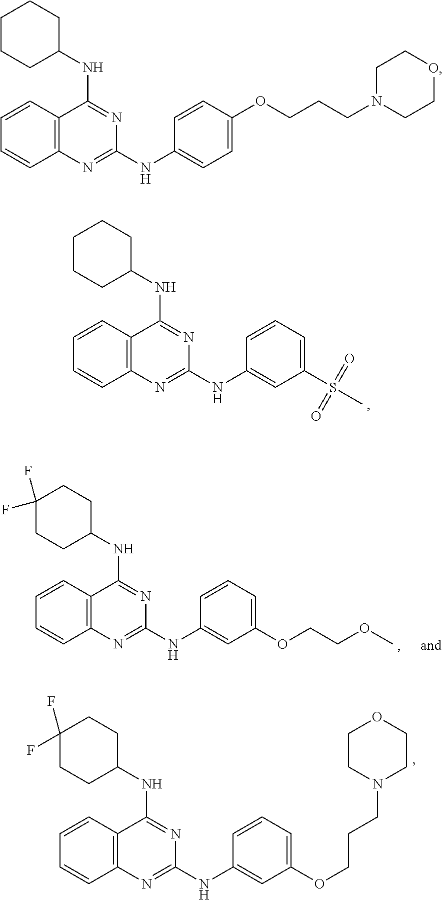 [SMILES: c1ccc2c(NC3CCCCC3)nc(Nc3ccc(OCCCN4CCOCC4)cc3)nc2c1 ; CS(=O)(=O)c1cccc(Nc2nc(NC3CCCCC3)c3ccccc3n2)c1 ; COCCOc1cccc(Nc2nc(NC3CCC(F)(F)CC3)c3ccccc3n2)c1 ; FC1(F)CCC(Nc2nc(Nc3cccc(OCCCN4CCOCC4)c3)nc3ccccc23)CC1]

and combinations thereof.

Embodiment 22 provides a compound of formula (2a) or salt, solvate, isotopologue, stereoisomer, tautomer, and/or any mixture thereof:

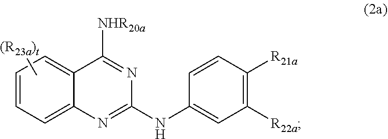

wherein:
R₂₀ₐ is selected from the group consisting of C₃-C₈ cycloalkyl and C₄-C₇ heterocycloalkyl;
R₂₁ₐ is F;
R₂₂ₐ is F;
each occurrence of R₂₃ₐ is independently selected from the group consisting of H, C₁-C₆ alkyl, —C(R₂₅ₐ)₃, F, Cl, Br, and I, wherein at least one R₂₃ₐ is selected from the group consisting of C₁-C₆ alkyl, —C(R₂₅ₐ)₃, F, Cl, Br, and I;
each occurrence of R₂₅ₐ is independently selected from the group consisting of F, Cl, Br, and I; and
t is 4.

Embodiment 23 provides the compound of Embodiment 22, wherein at least one of the following applies:
(i) R₂₀ is selected from the group consisting of cyclohexane,

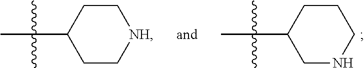 [SMILES: *C1CCNCC1 ; *C1CCCNC1]

and
(ii) three of R₂₃ are H and one R₂₃ is selected from the group consisting of F, Cl, Br, CH₃, and CF₃.

Embodiment 24 provides the compound of Embodiment 22, wherein the compound is selected from the group consisting of:

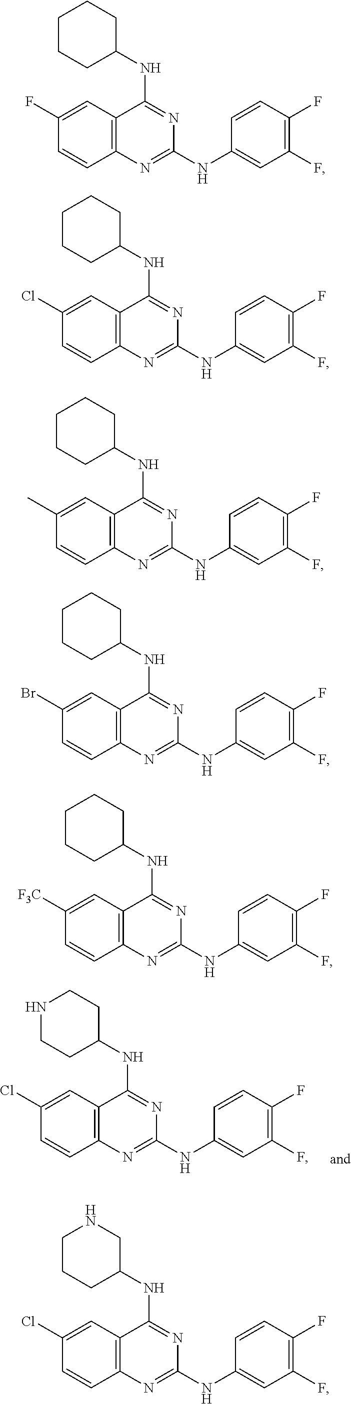

and combinations thereof.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A method of treating or ameliorating airway disease in a subject, the method comprising at least one of the following:
administering to the subject a therapeutically effective amount of a compound of formula (1), or a pharmaceutically acceptable salt, solvate, isotopologue, stereoisomer, tautomer, or any mixture thereof: 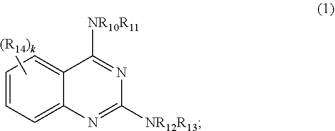
wherein:
R₁₀ and R₁₁ are each independently selected from the group consisting of H and optionally substituted C₃-C₈ cycloalkyl;
R₁₂ and R₁₃ are each independently selected from the group consisting of H; C₁-C₆ alkyl;
C₁-C₆ alkoxy; optionally substituted C₃-C₈ cycloalkyl; —(CH₂)ₙO(CH₂)ₘCH₃; optionally monosubstituted phenyl wherein the optional substituent is selected from the group consisting of F, Br, NO₂, CF₃, —S(═O)₂CH₃, —O(CH₂)ₚOCH₃, and 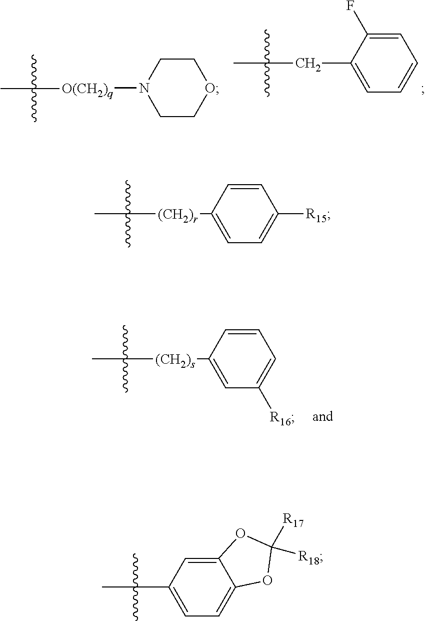
or
R₁₂ and R₁₃ combine with the nitrogen to which they are attached to form 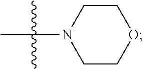 [SMILES: *N1CCOCC1]
each occurrence of R₁₄ is independently selected from the group consisting of H, C₁-C₆ alkyl, F, Br, Cl, and I;
R₁₅, R₁₇, and R₁₈ are each independently selected from the group consisting of C₁-C₆ alkyl, F, Br, Cl, and I;
R₁₆ is selected from the group consisting of F, Br, Cl, I, and —C(R₁₉)₃;
R₁₉ is selected from the group consisting of F, Br, Cl, and I;
k is 4;
m, n, p, and q are each independently selected from the group consisting of 1, 2, and 3; and
r and s are each independently selected from the group consisting of 0 and 1;
(ii) administering to the subject a therapeutically effective amount of a compound of formula (2), or a pharmaceutically acceptable salt, solvate, isotopologue, stereoisomer, tautomer, or any mixture thereof: 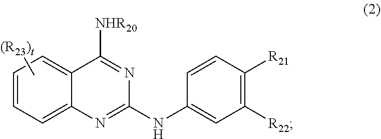
wherein:
R₂₀ is selected from the group consisting of C₃-C₈ cycloalkyl and C₄-C₇ heterocycloalkyl;
R₂₁ and R₂₂ are each independently selected from the group consisting of F, Cl, Br, I, C₁-C₆ alkoxy, and —SC(R₂₄)₃, with the proviso that R₂₁ is C₁-C₆ alkoxy if and only if R₂₂ is C₁-C₆ alkoxy;
each occurrence R₂₃ is independently selected from the group consisting of H, C₁-C₆ alkyl, —C(R₂₅)₃, F, Cl, Br, and I;
each occurrence of R₂₄ and R₂₅ is independently selected from the group consisting of F, Cl, Br, and I; and
t is 4; or
(iii) administering to the subject a therapeutically effective amount of a compound of formula (3), or a pharmaceutically acceptable salt, solvate, isotopologue, stereoisomer, tautomer, or any mixture thereof: 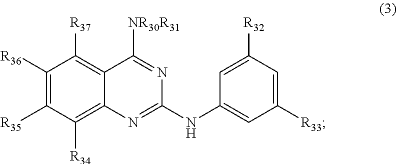
wherein:
R₃₀ and R₃₁ are each independently selected from the group consisting of: H; C₁-C₆ linear alkyl optionally substituted with at least one substituent selected from the group consisting of —NR₃₈R₃₉, C₃-C₈ cycloalkyl, phenyl, and OH; C₃-C₁₂ cycloalkyl optionally substituted with at least one substituent selected from the group consisting of C₁-C₆ alkyl, C═O, and —NR₃₈R₃₉, wherein the —NR₃₈R₃₉ is optionally substituted with C₄-C₇ heterocycloalkyl; C₄-C₇ heterocycloalkyl optionally substituted with at least one substituent selected from the group consisting of phenyl, benzyl, CH₂CH₂OCH₃, and C(═O)R₄₁; phenyl substituted with one or more substituents selected from the group consisting of F, Br, I, OH, C₁-C₆ alkyl, C₁-C₆ alkoxy, —N(R₄₀)₂, and —C(═O)R₄₁; 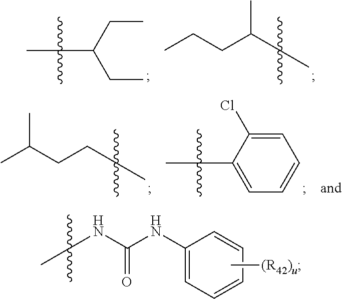
R₃₂ and R₃₃ are each independently selected from the group consisting of F, Cl, Br, and I;
R₃₄ and R₃₆ are each independently selected from the group consisting of H, C₁-C₆ alkyl, C₁-C₆ alkoxy, F, Cl, Br, and I;
R₃₅ and R₃₇ are each independently selected from the group consisting of H and C₁-C₆ alkyl;
R₃₈ and R₃₉ are each independently selected from the group consisting of H, C₁-C₆ alkyl, and C₆-C₁₂ aryl;
each R₄₀ is selected from the group consisting of H and C₁-C₆ linear alkyl;
R₄₁ is selected from the group consisting of H, C₁-C₆ alkyl, and C₁-C₆ alkoxy;
R₄₂ is selected from the group consisting of F, Cl, Br, and I; and
u is 1, 2, 3, 4, or 5.

2. The method of claim 1, wherein the compound comprises a compound of formula (1) wherein one of the following applies:
R₁₀ and R₁₁ are each H; or
(ii) R₁₀ is H and R₁₁ is selected from the group consisting of

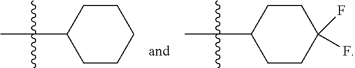 [SMILES: *C1CCCCC1 ; *C1CCC(F)(F)CC1]

3. The method of claim 1, wherein the compound comprises a compound of formula (1) wherein one of the following applies:
R₁₂ and R₁₃ are each H;
(ii) R₁₂ and R₁₃ are each CH₂CH₃;
(iii) R₁₂ is CH₃ and R₁₃ is phenyl;
(iii) R₁₂ is H and R₁₃ is selected from the group consisting of 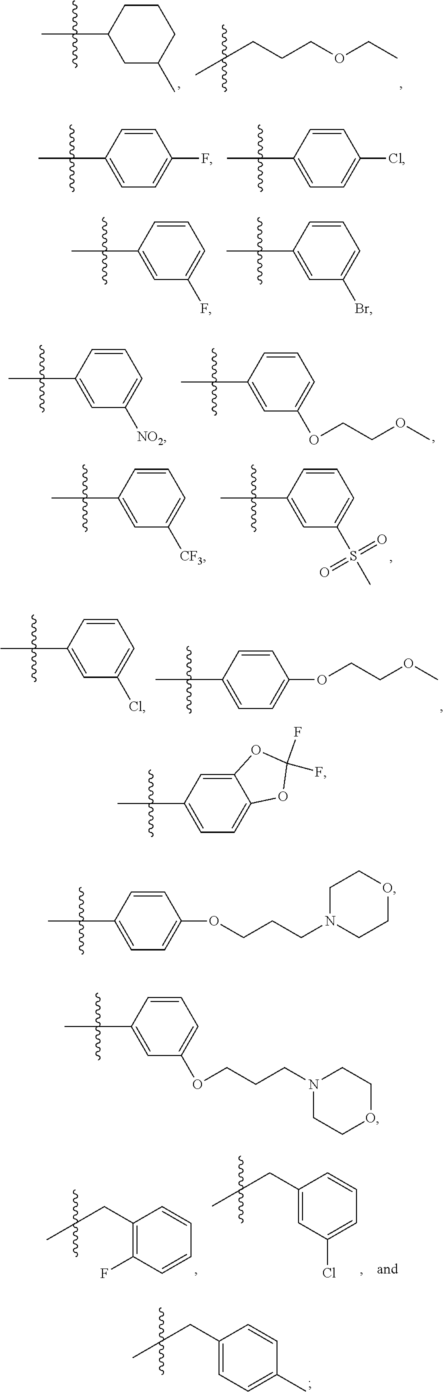
or
(iv) R₁₂ and R₁₃ combine with the nitrogen to which they are attached to form

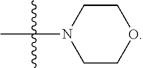 [SMILES: *N1CCOCC1]

4. The method of claim 1, wherein the compound comprises a compound of formula (1) wherein one of the following applies:
(i) each R₁₄ is independently H; or
(ii) three R₁₄ are H and one R₁₄ is selected from the group consisting of Cl and F.

5. The method of claim 1, wherein the compound comprises a compound of formula (1) selected from the group consisting of: 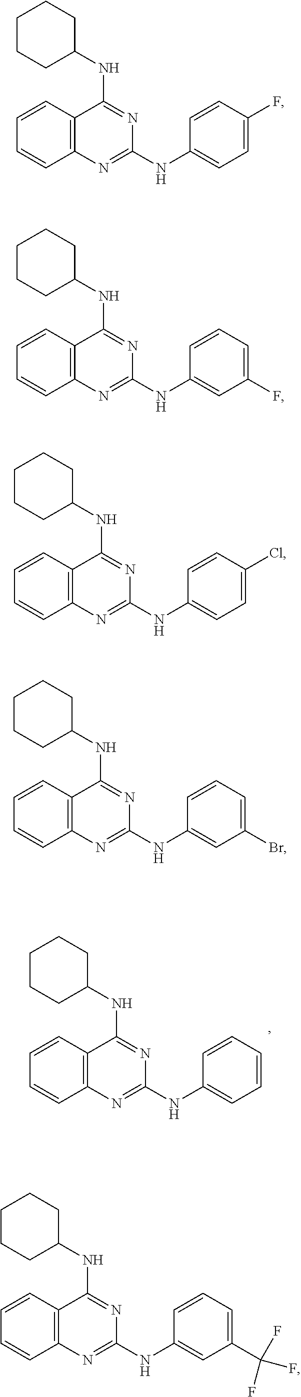 [SMILES: Fc1ccc(Nc2nc(NC3CCCCC3)c3ccccc3n2)cc1 ; Fc1cccc(Nc2nc(NC3CCCCC3)c3ccccc3n2)c1 ; Clc1ccc(Nc2nc(NC3CCCCC3)c3ccccc3n2)cc1 ; Brc1cccc(Nc2nc(NC3CCCCC3)c3ccccc3n2)c1 ; c1ccc(Nc2nc(NC3CCCCC3)c3ccccc3n2)cc1 ; FC(F)(F)c1cccc(Nc2nc(NC3CCCCC3)c3ccccc3n2)c1] 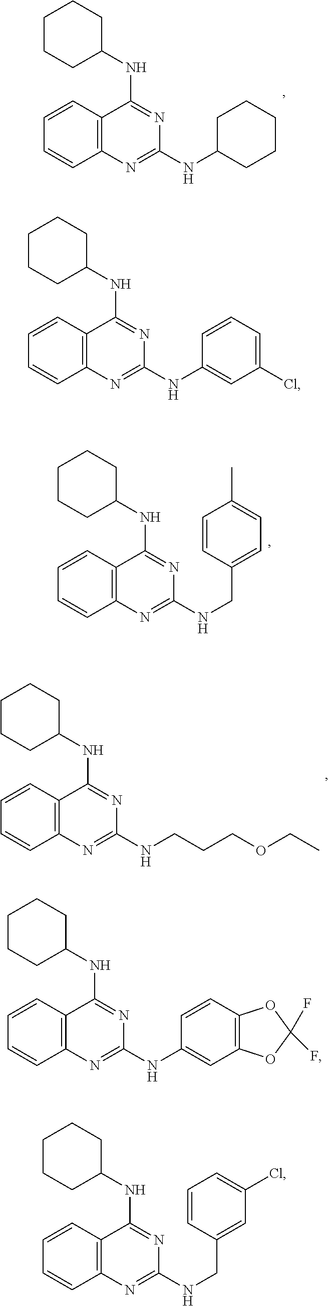 [SMILES: c1ccc2c(NC3CCCCC3)nc(NC3CCCCC3)nc2c1 ; Clc1cccc(Nc2nc(NC3CCCCC3)c3ccccc3n2)c1 ; Cc1ccc(CNc2nc(NC3CCCCC3)c3ccccc3n2)cc1 ; CCOCCCNc1nc(NC2CCCCC2)c2ccccc2n1 ; FC1(F)Oc2ccc(Nc3nc(NC4CCCCC4)c4ccccc4n3)cc2O1 ; Clc1cccc(CNc2nc(NC3CCCCC3)c3ccccc3n2)c1] 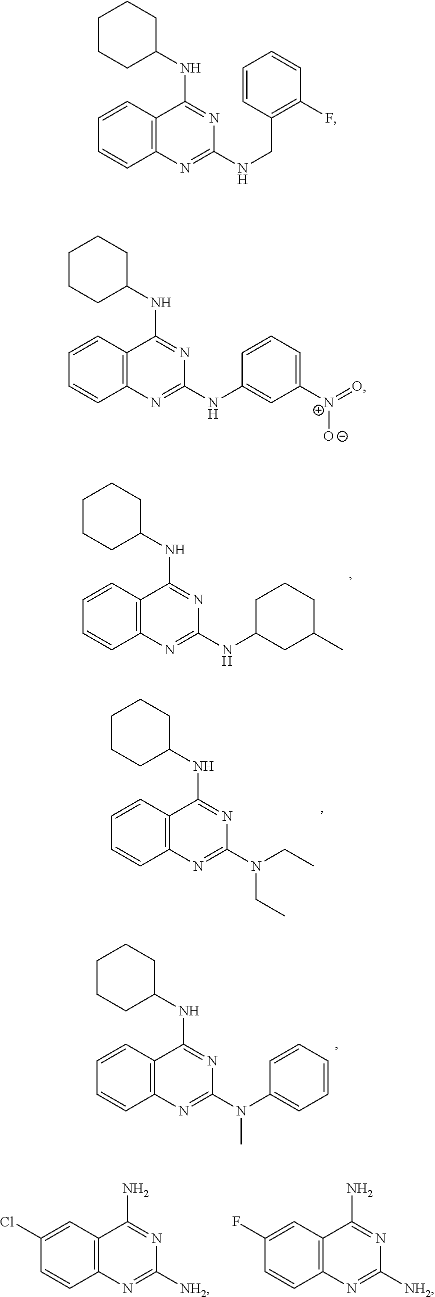 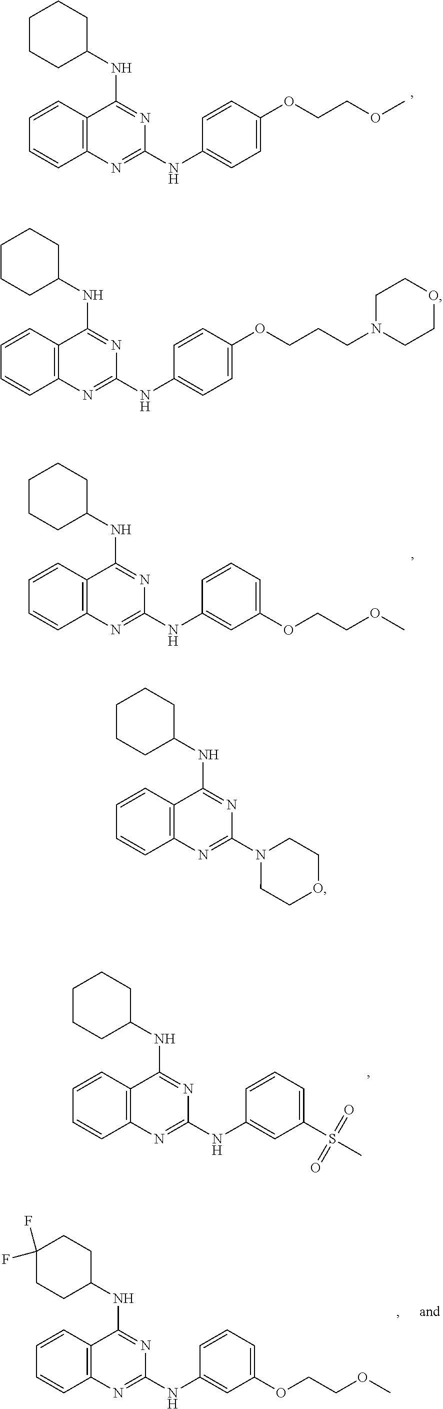 [SMILES: COCCOc1ccc(Nc2nc(NC3CCCCC3)c3ccccc3n2)cc1 ; c1ccc2c(NC3CCCCC3)nc(Nc3ccc(OCCCN4CCOCC4)cc3)nc2c1 ; COCCOc1cccc(Nc2nc(NC3CCCCC3)c3ccccc3n2)c1 ; c1ccc2c(NC3CCCCC3)nc(N3CCOCC3)nc2c1 ; CS(=O)(=O)c1cccc(Nc2nc(NC3CCCCC3)c3ccccc3n2)c1 ; COCCOc1cccc(Nc2nc(NC3CCC(F)(F)CC3)c3ccccc3n2)c1] 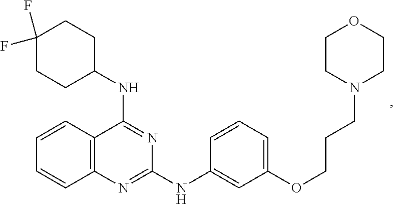 [SMILES: FC1(F)CCC(Nc2nc(Nc3cccc(OCCCN4CCOCC4)c3)nc3ccccc23)CC1]
and combinations thereof.

6. The method of claim 1, wherein the compound comprises a compound of formula (2) wherein R₂₀ is selected from the group consisting of cyclohexane,

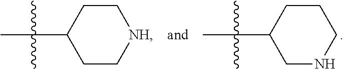 [SMILES: *C1CCNCC1 ; "and" ; *C1CCCNC1]

7. The method of claim 1, wherein the compound comprises a compound of formula (2) wherein one of the following applies:
(i) each of R₂₁ is F and R₂₂ is F;
(ii) each of R₂₁ is C₁ and R₂₂ is Cl;
(iii) each of R₂₁ is OCH₃ and R₂₂ is OCH₃;
(iv) R₂₁ is F and R₂₂ is Cl;
(v) R₂₁ is C₁ and R₂₂ is F;
(vi) R₂₁ is C₁ and R₂₂ is SCF₃; or
(vii) R₂₁ is SCF₃ and R₂₂ is Cl.

8. The method of claim 1, wherein the compound comprises a compound of formula (2) wherein one of the following applies:
(i) each of R₂₃ is H; or
(ii) three of R₂₃ are H and one R₂₃ is selected from the group consisting of F, Cl, Br, CH₃, and CF₃.

9. The method of claim 1, wherein the compound comprises a compound of formula (2) selected from the group consisting of: 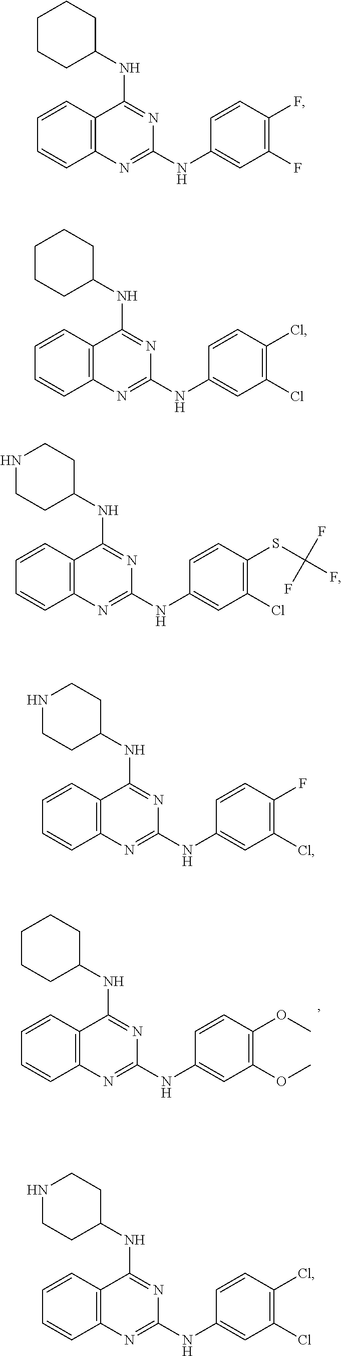 [SMILES: Fc1ccc(Nc2nc(NC3CCCCC3)c3ccccc3n2)cc1F ; Clc1ccc(Nc2nc(NC3CCCCC3)c3ccccc3n2)cc1Cl ; FC(F)(F)Sc1ccc(Nc2nc(NC3CCNCC3)c3ccccc3n2)cc1Cl ; Fc1ccc(Nc2nc(NC3CCNCC3)c3ccccc3n2)cc1Cl ; COc1ccc(Nc2nc(NC3CCCCC3)c3ccccc3n2)cc1OC ; Clc1ccc(Nc2nc(NC3CCNCC3)c3ccccc3n2)cc1Cl] 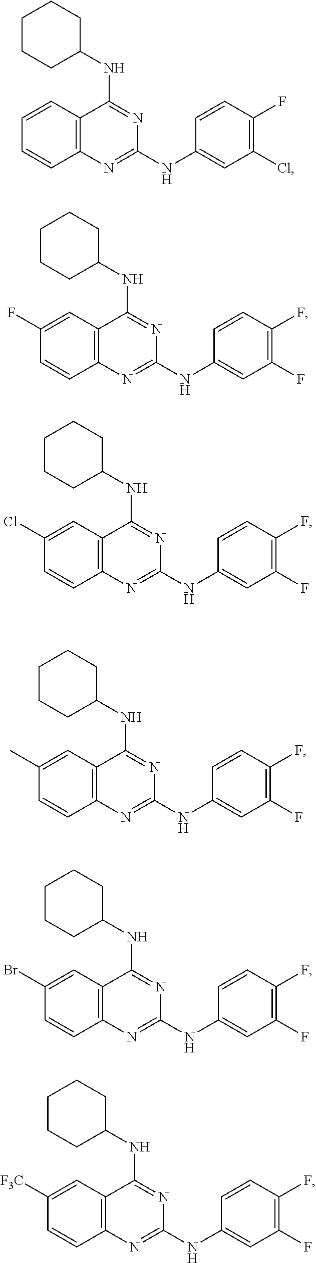 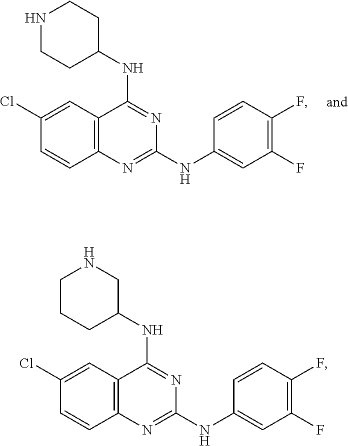 [SMILES: Fc1ccc(Nc2nc(NC3CCNCC3)c3cc(Cl)ccc3n2)cc1F ; Fc1ccc(Nc2nc(NC3CCCNC3)c3cc(Cl)ccc3n2)cc1F]
and combinations thereof.

10. The method of claim 1, wherein the compound comprises a compound of formula (3) wherein one of the following applies:
(i) R₃₀ and R₃₁ are each H;
(ii) R₃₀ and R₃₁ are each —CH₂CH₃; or
(iii) R₃₀ is H and R₃₁ is selected from the group consisting of —CH₂CH₃, —(CH₂)₄CH₃, —(CH₂)₂₀H, —(CH₂)phenyl, —(CH₂)₂phenyl, —(CH₂)cyclohexane, —(CH₂)₂cyclohexane, —(CH₂)₃NH₂, —(CH₂)₃N(CH₃)₂, 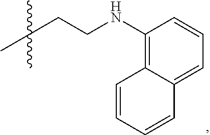 [SMILES: *CCNc1cccc2ccccc12]
cyclopentane, cyclohexane, cycloheptane, adamantyl, 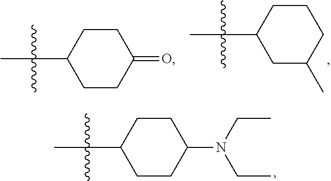
piperidine, 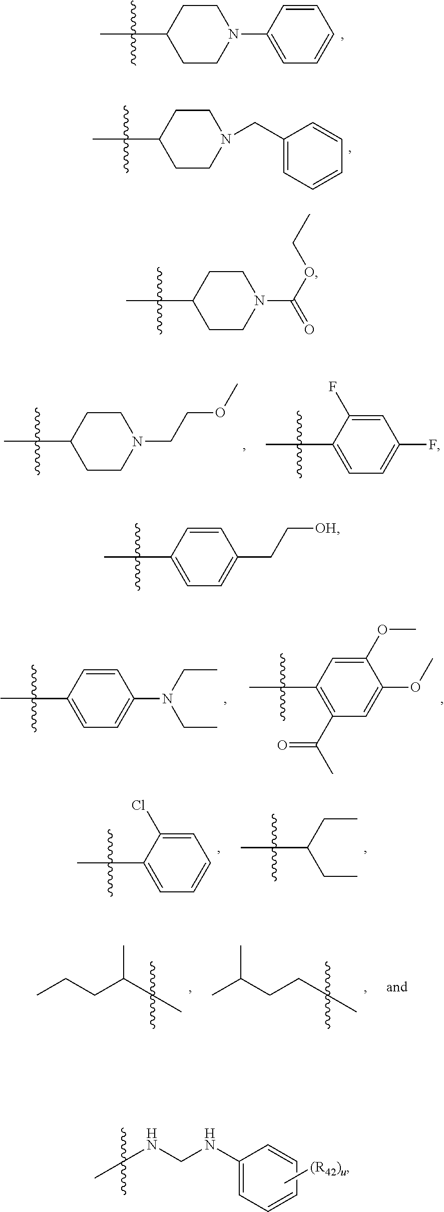
wherein R₄₂ is F and u is 1.

11. The method of claim 1, wherein the compound comprises a compound of formula (3) wherein one of the following applies:
(i) R₃₂ is Cl and R₃₃ is Cl;
(ii) R₃₂ is F and R₃₃ is F; or
(iii) R₃₂ is F and R₃₃ is I.

12. The method of claim 1, wherein the compound comprises a compound of formula (3) wherein at least one of the following applies:
(i) R₃₄ is selected from the group consisting of H and CH₃;
(ii) R₃₅ is selected from the group consisting of H and CH₃;
(iii) R₃₆ is selected from the group consisting of H, CH₃, F, and Cl; and
(iv) R₃₇ is H.

13. The method of claim 1, wherein the compound comprises a compound of formula (3) selected from the group consisting of: 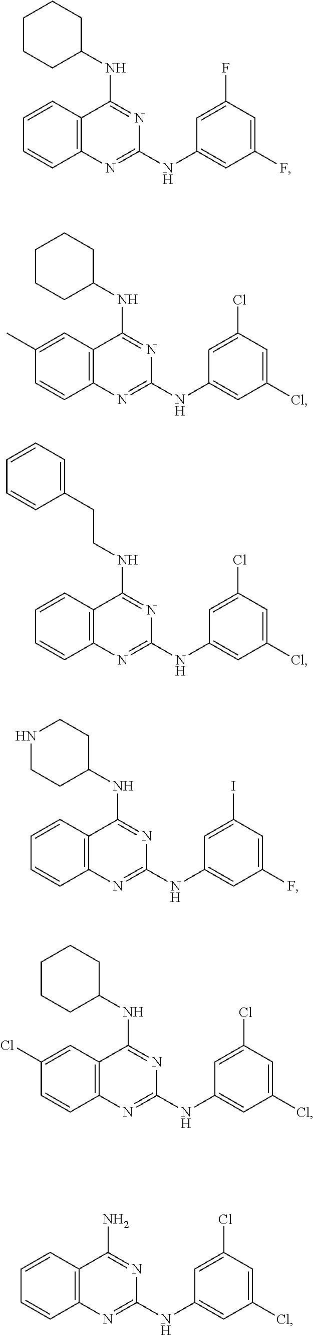 [SMILES: Fc1cc(F)cc(Nc2nc(NC3CCCCC3)c3ccccc3n2)c1 ; Cc1ccc2nc(Nc3cc(Cl)cc(Cl)c3)nc(NC3CCCCC3)c2c1 ; Clc1cc(Cl)cc(Nc2nc(NCCc3ccccc3)c3ccccc3n2)c1 ; Fc1cc(I)cc(Nc2nc(NC3CCNCC3)c3ccccc3n2)c1 ; Clc1cc(Cl)cc(Nc2nc(NC3CCCCC3)c3cc(Cl)ccc3n2)c1 ; Nc1nc(Nc2cc(Cl)cc(Cl)c2)nc2ccccc12] 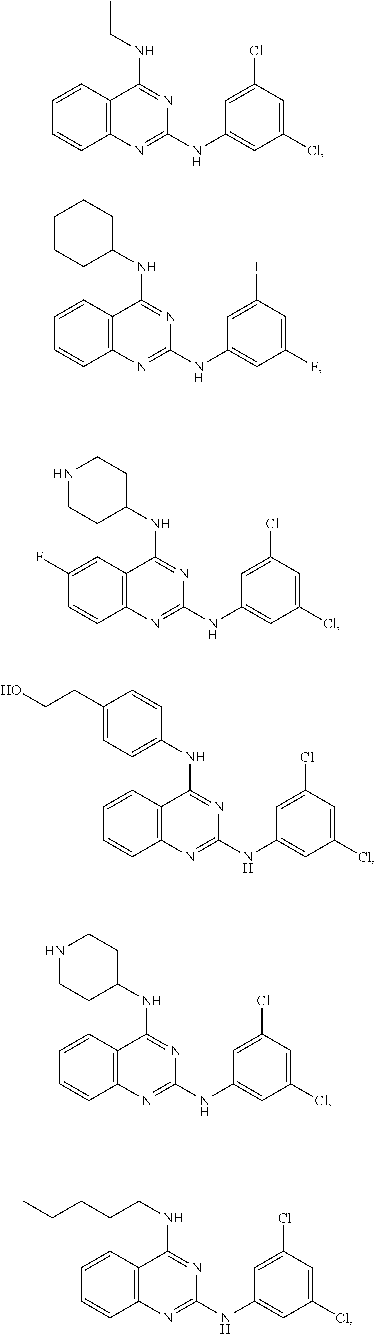 [SMILES: CCNc1nc(Nc2cc(Cl)cc(Cl)c2)nc2ccccc12 ; Fc1cc(I)cc(Nc2nc(NC3CCCCC3)c3ccccc3n2)c1 ; Fc1ccc2nc(Nc3cc(Cl)cc(Cl)c3)nc(NC3CCNCC3)c2c1 ; OCCc1ccc(Nc2nc(Nc3cc(Cl)cc(Cl)c3)nc3ccccc23)cc1 ; Clc1cc(Cl)cc(Nc2nc(NC3CCNCC3)c3ccccc3n2)c1 ; CCCCCNc1nc(Nc2cc(Cl)cc(Cl)c2)nc2ccccc12] 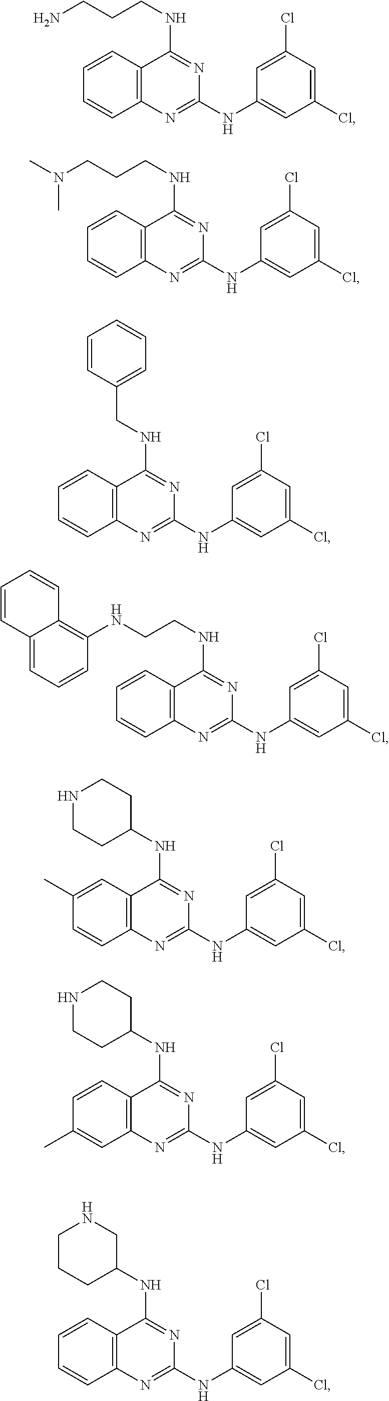 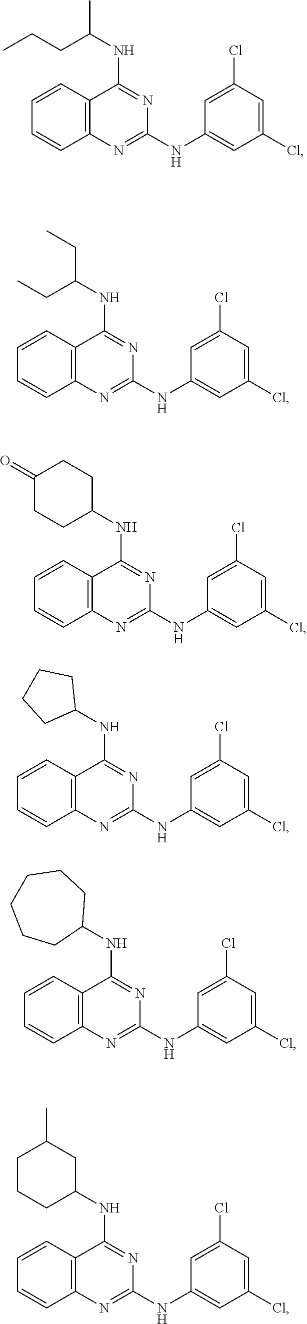 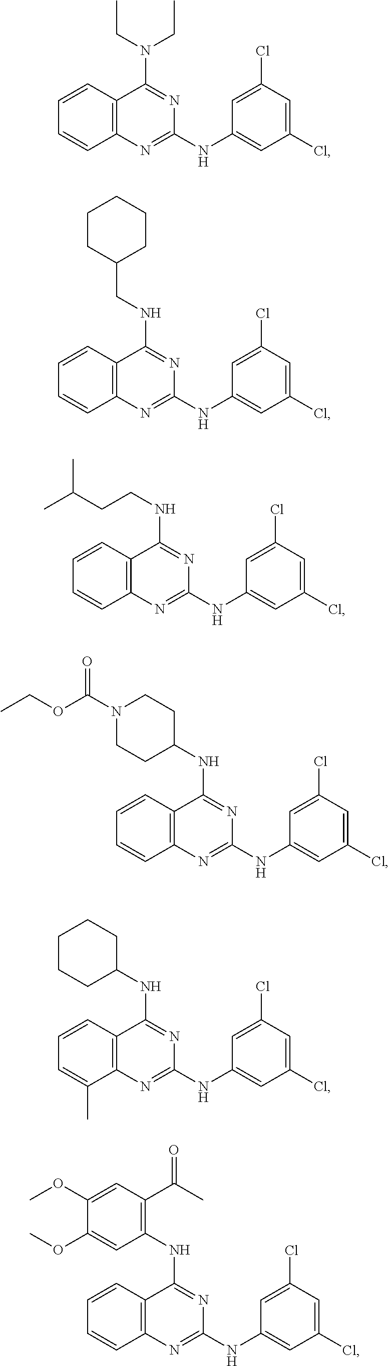 [SMILES: CCN(CC)c1nc(Nc2cc(Cl)cc(Cl)c2)nc2ccccc12 ; Clc1cc(Cl)cc(Nc2nc(NCC3CCCCC3)c3ccccc3n2)c1 ; CC(C)CCNc1nc(Nc2cc(Cl)cc(Cl)c2)nc2ccccc12 ; CCOC(=O)N1CCC(Nc2nc(Nc3cc(Cl)cc(Cl)c3)nc3ccccc23)CC1 ; Cc1cccc2c(NC3CCCCC3)nc(Nc3cc(Cl)cc(Cl)c3)nc12 ; COc1cc(Nc2nc(Nc3cc(Cl)cc(Cl)c3)nc3ccccc23)c(C(C)=O)cc1OC] 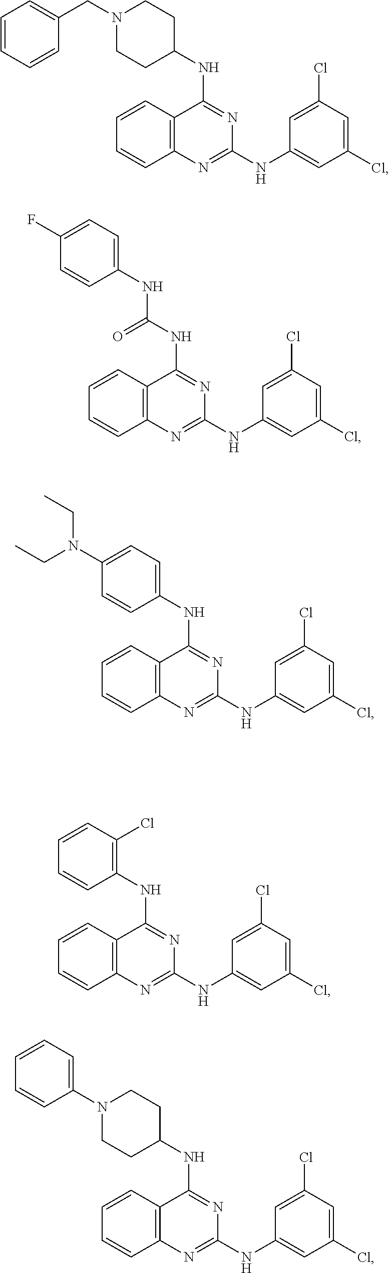 [SMILES: Clc1cc(Cl)cc(Nc2nc(NC3CCN(Cc4ccccc4)CC3)c3ccccc3n2)c1 ; O=C(Nc1ccc(F)cc1)Nc1nc(Nc2cc(Cl)cc(Cl)c2)nc2ccccc12 ; CCN(CC)c1ccc(Nc2nc(Nc3cc(Cl)cc(Cl)c3)nc3ccccc23)cc1 ; Clc1cc(Cl)cc(Nc2nc(Nc3ccccc3Cl)c3ccccc3n2)c1 ; Clc1cc(Cl)cc(Nc2nc(NC3CCN(c4ccccc4)CC3)c3ccccc3n2)c1] 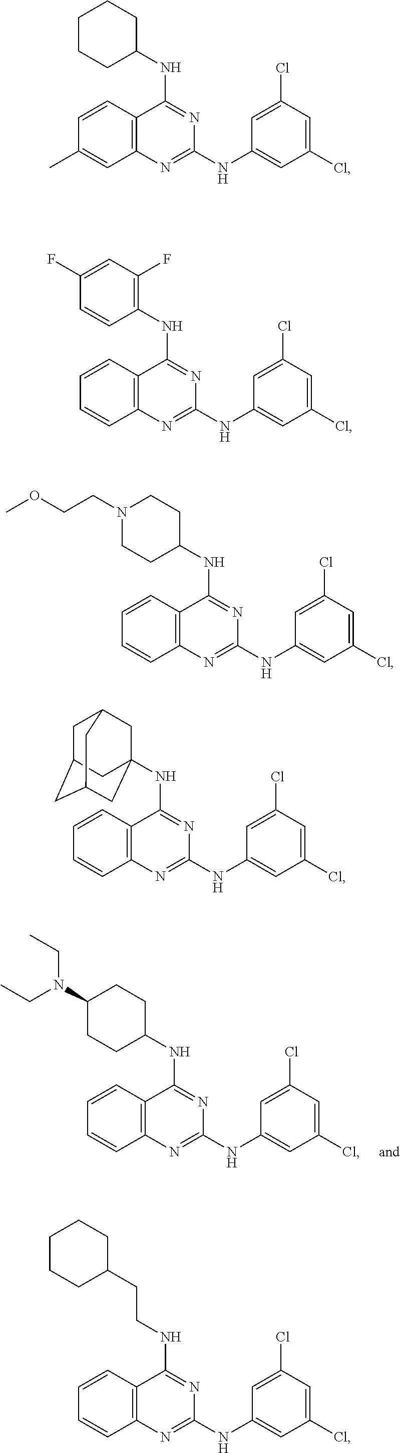
and combinations thereof.

14. The method of claim 1, wherein at least one of the following applies:
(a) the airway disease is asthma or chronic obstructive pulmonary disease;
(b) the compound is administered to the subject through a route comprising nasal, inhalational, intratracheal, intrapulmonary, intrabronchial, topical, oral, buccal, rectal, pleural, peritoneal, vaginal, intramuscular, subcutaneous, transdermal, epidural, otic, intraocular, intrathecal, or intravenous;
(c) the compound is formulated in a pharmaceutical composition; and
(d) the subject is a mammal, optionally wherein the mammal is a human.

15-18. (canceled)

19. A compound of formula (1a), or a salt, solvate, isotopologue, stereoisomer, tautomer, or any mixture thereof: 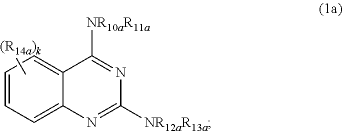
wherein:
R₁₀ₐ and R₁₁ₐ are each independently selected from the group consisting of H and optionally substituted C₃-C₈ cycloalkyl;
R₁₂ₐ is H;
R₁₃ₐ is a monosubstituted phenyl wherein the substituent is selected from —S(═O)₂CH₃, —(CH₂)ₙO(CH₂)ₘCH₃, and 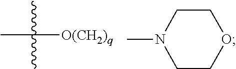
each occurrence of R₁₄ₐ is H;
k is 4; and
m, n, and q are each independently 1, 2, or 3;
with the proviso that, if R₁₃ₐ is phenyl monosubstituted with —(CH₂)ₙO(CH₂)ₘCH₃, then R₁₁ₐ is substituted C₃-C₈ cycloalkyl.

20. The compound of claim 19, wherein at least one of the following applies:
(i) R₁₀ₐ is H and R₁₁ₐ is selected from the group consisting of 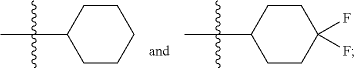 [SMILES: *C1CCCCC1 ; *C1CCC(F)(F)CC1]
(ii) R₁₃ₐ is selected from the group consisting of 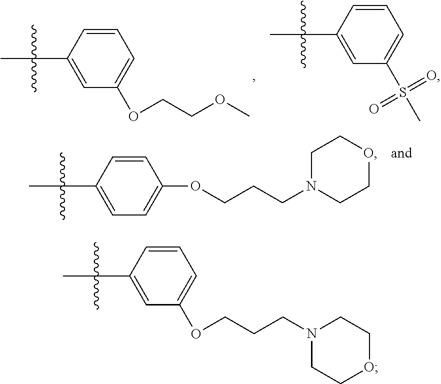
and
(iii) R₁₃ₐ is phenyl monosubstituted with —(CH₂)ₙO(CH₂)ₘCH₃, and R₁₁ₐ is

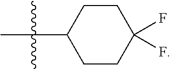 [SMILES: *C1CCC(F)(F)CC1]

21. The compound of claim 19, wherein the compound is selected from the group consisting of: 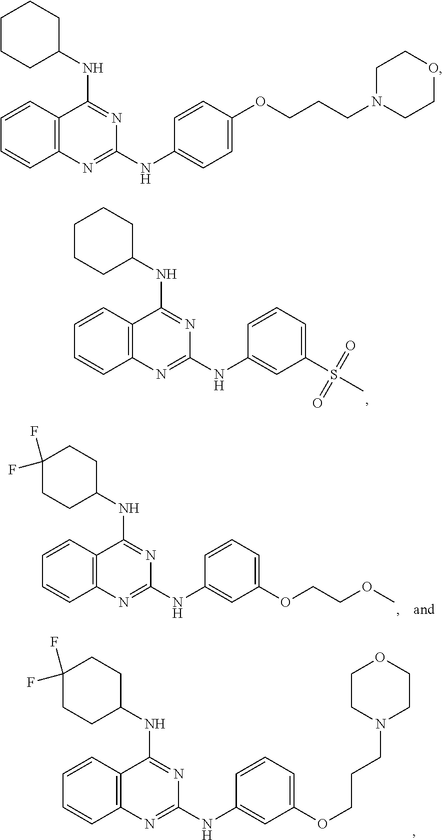
and combinations thereof.

22. A compound of formula (2a) or salt, solvate, isotopologue, stereoisomer, tautomer, or any mixture thereof: 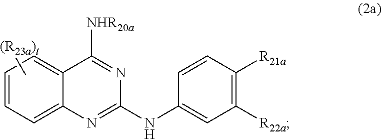
wherein:
R₂₀ₐ is selected from the group consisting of C₃-C₈ cycloalkyl and C₄-C₇ heterocycloalkyl;
R₂₁ₐ is F;
R₂₂ₐ is F;
each occurrence of R₂₃ₐ is independently selected from the group consisting of H, C₁-C₆ alkyl, —C(R₂₅ₐ)₃, F, Cl, Br, and I, wherein at least one R₂₃ₐ is selected from the group consisting of C₁-C₆ alkyl, —C(R₂₅ₐ)₃, F, Cl, Br, and I;
each occurrence of R₂₅ₐ is independently selected from the group consisting of F, Cl, Br, and I; and
t is 4.

23. The compound of claim 22, wherein at least one of the following applies:
R₂₀ is selected from the group consisting of cyclohexane, 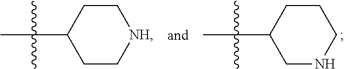 [SMILES: *C1CCNCC1 ; "and" ; *C1CCCNC1]
and
(ii) three of R₂₃ are H and one R₂₃ is selected from the group consisting of F, Cl, Br, CH₃, and CF₃.

24. The compound of claim 22, wherein the compound is selected from the group consisting of: 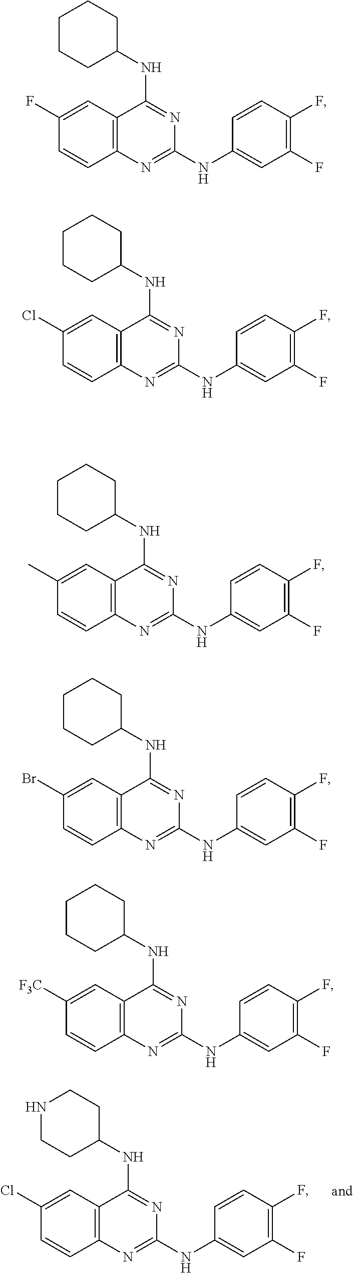 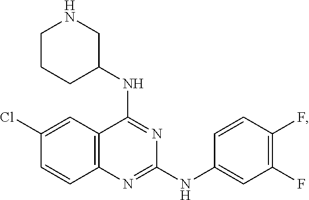 [SMILES: Fc1ccc(Nc2nc(NC3CCCNC3)c3cc(Cl)ccc3n2)cc1F]
and combinations thereof.
